(19) United States
(12) Patent Application Publication
(10) Pub. No.: US 2009/0177450 A1
(43) Pub. Date: Jul. 9, 2009

(54) SYSTEMS AND METHODS FOR PREDICTING RESPONSE OF BIOLOGICAL SAMPLES

(75) Inventors: Joe W. Gray, San Francisco, CA (US); Debopriya Das, Albany, CA (US); Nicholas Wang, San Jose, CA (US); Wen-Lin Kuo, San Ramon, CA (US); Paul Spellman, Benicia, CA (US)

Correspondence Address: KNOBBE MARTENS OLSON & BEAR LLP, 2040 MAIN STREET, FOURTEENTH FLOOR, IRVINE, CA, 92614, US

(73) Assignee: Lawrence Berkeley National Laboratory, Berkeley, CA (US)

(21) Appl. No.: 12/333,192

(22) Filed: Dec. 11, 2008

Related U.S. Application Data
(63) Continuation-in-part of application No. PCT/US2008/059176, filed on Apr. 2, 2008.
(60) Provisional application No. 61/013,278, filed on Dec. 12, 2007.

(51) Int. Cl. G06F 17/17; C12Q 1/68; G06F 1/02; G06G 7/58; G06N 5/02

(52) U.S. Cl. 703/2; 435/6; 708/270; 703/11; 702/19; 706/46

(57) ABSTRACT

Embodiments relate to genomic technologies using adaptive spline analysis that predict responses of cancer cells. For example, responses of cancer cells to specific medications and/or treatments may be predicted based on adaptive linear spline analyses.

### CROSS-REFERENCE TO RELATED APPLICATION

This application claims the benefit of U.S. Provisional Patent Application 61/013278 filed Dec. 12, 2007 and is a continuation-in-part of PCT Patent Application PCT/US2008/059176 filed Apr. 2, 2008 designating the United States and published in the English language. The contents of each of these related applications are hereby incorporated by reference in their entirety.

### STATEMENT REGARDING FEDERALLY SPONSORED R&D

This invention was made with government support under Grant Number 5U54CA112970-04 awarded by the National Cancer Institute, and under Contract No. DE-AC02-05CH11231 awarded by the Department of Energy. The government has certain rights in the invention.

### PARTIES OF JOINT RESEARCH AGREEMENT

This invention was partially funded through Work for Others Agreement LB06-002417 between The Regents of the University of California through Ernest Orlando Lawrence Berkeley National Laboratory under its U.S. Department of Energy Contract No. DE-AC02-05CH11231 and GlaxoSmithKline, Inc.

### BACKGROUND OF THE INVENTION

1. Field of the Invention

Embodiments relate to genomic technologies using spline functions that predict physiological responses of cells. For example, responses of cancer cells to specific medications and/or treatments may be predicted based on adaptive linear spline analyses.

2. Description of the Related Art

Over 12 million new cancer diagnoses were made and approximately 7.6 million cancer deaths occurred in 2007. The drug industry for cancer, America's second-biggest killer behind heart disease, is growing rapidly. Cancer drug sales are expected to grow to more than $100 billion by 2010. Furthermore, the R&D cost for discovering a new therapeutic agent is growing exponentially, largely due to ineffective clinical trials.

Due to the heterogeneity within and across cancers, a single treatment may be effective for some cancer patients and not for others. Genome scale analyses of multiple types of cancers have made it evident that these disease cells manifest a variety of genomic, transcriptional and translational defects that influence disease pathophysiology and response to therapy. In concordance with our increased understanding of the complex molecular biology of cancer, rational design of therapeutics targeted to key oncogenes has been adopted. However, even among patients selected for these therapies, based on expression of the target genes, less than half exhibit clinical response or benefit from therapy.

Identification of molecular predictors of response to therapeutic agents is an increasingly important aspect of efforts to individualize treatment of cancer and other diseases, and constitutes a cornerstone of personalized medicine. Ideally, such molecular predictors can be identified sufficiently early in the drug development process to guide the introduction of new drugs in early clinical trials. It is anticipated that stratifying patient populations using predictive markers will dramatically reduce the cost of drug development and ineffective therapies. Thus, there is a need for improved individualization of patient treatment in order to improve treatment efficacy.

### SUMMARY OF THE INVENTION

In some embodiments, a method for predicting a physiological response of a patient to a treatment is provided, the method comprising: providing a sample physiological response for each of a plurality of training samples to the treatment; providing a quantification value of a marker for each of the plurality of training samples; determining a predictive model relating the sample physiological responses to the quantification values, the model comprising a spline function; and predicting a physiological response of a biological sample to the treatment using the model.

In some embodiments, a system for relating quantification values of markers to physiological response is provided, the system comprising an input component configured to receive input data for each of a plurality of samples, the input data comprising a physiological response to a treatment and a quantification value of a marker in the sample; a univariate model generator configured to determine a univariate model relating the physiological response to the quantification value using a spline-based analysis; and an output device configured to output one or more variables or equations related to the univariate model.

In some embodiments, a method for identifying a marker influencing a physiological response of a sample is provided, the method comprising: providing a physiological response for each of a plurality of training samples to the treatment; providing a value of each of a plurality of markers for each of the plurality of training samples; determining a plurality of univariate models, each model relating the physiological responses to values of one of the plurality the marker, each model comprising a spline function; and identifying a marker influencing the physiological response based on the plurality of univariate models.

### BRIEF DESCRIPTION OF THE DRAWINGS

FIG. 1 shows a process for developing a model of a response to a therapeutic treatment.

FIG. 2 shows a schematic of the hierarchical modeling approach. Univariate models, {fₓ(xᵢ)}, are constructed for each dataset at the first level of the hierarchy; multivariate models, {F_{X}(x₁, x₂ , K)}, that combine the univariate predictors are built for each dataset separately at the next level; the final predictor of response, H({cᵢ}, {gᵢ}, {pᵢ}), which integrates all multivariate models from various platforms is obtained at the final level of hierarchy.

FIG. 3 shows a system for determining a physiological prediction.

FIG. 4 shows an adaptive linear spline fits to simulated data sets with (a) linear variation, and 2-class structures where (b) neither class has a significant internal variation, (c) only one class has internal variation, and (d) both classes have internal variation.

FIG. 5 shows results of simulations. The predictive accuracy of different univariate tests for various types of underlying models: (a) two classes with different constant log(GI₅₀) in each class, (b) linear correlation with expression, (c) two classes, one class with constant log(GI₅₀) and the other with linear variation, (d) two classes, each with a different linear correlation. Results are displayed for four different tests: t-test (diamonds), linear fit (circles), single linear spline fit (x's) and adaptive spline fit (squares). The left panel (left axis) shows the goodness of fit (discrimination for t-test) for the best marker for each of the tests, reflecting its predictive power. The right panel shows the similarity between the expression profile of the best marker for each test and that of the original marker used to build the model. The triangles in the left panel record RSS_{original}/RSS_{final} (right axis) for adaptive spline fit, which is greater than 1 when there is overfitting. All data points reflect average over nᵢₜₑᵣ=20 iterations.

FIG. 6 shows 5-FU induced apoptosis in colon cancer cells. (a) Adaptive spline fit for the top mRNA predictor of apoptotic response, PDZD11 (p=2e-6, FDR =0.2%)—a novel marker revealed by this analysis. (b) Unsupervised hierarchical clustering of significant genes predictive of apoptosis reveals 3 distinct gene clusters: first cluster has high expression in one set of cell-lines and low expression in others, second cluster has linear variation, while the third cluster has a pattern complementary to the first one. (c) Leave-one-out cross-validation accuracy of the multivariate model using adaptive linear splines. Equation of the trendline: 0.55+0.32x (p=6.9e-08).

FIG. 7 shows sensitivity of breast cancer cells to Lapatinib. Measured GI₅₀ profile of 40 breast cancer cell-lines to Lapatinib. Cell-lines with positive ERBB2 status are shown with the unfilled bars.

FIG. 8 shows spline models of sensitivity to Lapatinib. (a) Unsupervised hierarchical clustering shows that significant mRNA markers automatically break up into two gene clusters: one cluster has high expression in one set of cell-lines and low expression in remaining cell-lines, while the other gene cluster has a complementary trend. (b) An example of how classes of cancer samples can be identified on the basis of a fitted adaptive linear spline. The left regions marks the cell-lines that are identified as sensitive (class=1), while the right region contains the cell-lines that are classified as resistant (class=−1). The cell-lines in the middle region have an undetermined class (class=0). (c) Unsupervised classification of cancer samples. Log(GI₅₀) (bars, left y-axis) and predicted class score (black curve, right y-axis) of cell-lines in the training set. The maximum GI₅₀ of the predicted sensitive class (left of dashed line) is lower than the minimum GI₅₀ of the predicted resistant class (right of dashed line), indicating clear separation characteristic of classification. This leads to a discriminatory dose concentration: log(GI₅₀)=−0.46 (arrow), distinctly different from the mean log(GI₅₀)=0.4. Only cell-lines with all 3 baseline molecular profiles were included in the analysis.

FIG. 9 shows ingenuity analysis of significant mRNA markers of response to Lapatinib. The most significant network, shown below, has ERBB2 as a major node. The shading indicate the p-value significance from low to high. The network is associated with 6 significant pathways (p<0.05): axonal guidance signaling, ephrin receptor signaling, protein ubiquitination, PPARα/RXRα activation, VEGF signaling and p53 signaling.

FIG. 10 shows leave-one-out cross-validation error (LOOCV) for model size selection. Plots of predicted vs measured log(GI₅₀) in LOOCV calculation of model size selection in weighted voting approach for (a) mRNA expression, (b) DNA copy number and (c) protein expression datasets.

FIG. 11 shows the strength of correlation between measured and predicted GI₅₀ of Lapatinib for the test set of 10 breast cancer cell-lines using weighted voting scheme (equation of trendline: y=0.09+0.63x, r=0.90, p=4.7e-04) (Inset shows the performance on the training set).

FIGS. 12A-B shows the progression-free survival in 49 ERBB2 positive tumors treated with Lapatinib plus Paclitaxel and 28 ERBB2 positive tumors treated with Paclitaxel plus placebo.

FIG. 13 is a bar chart showing quantitative responses of 40 breast cancer cell lines to Lapatinib treatment.

FIG. 14 is a line graph showing the Kaplan-Meier (KM) estimates for Lapatinib (a 4-anilinoquinazoline kinase inhibitor) and paclitaxel treatment of sensitivity-positive (sensitive) and sensitivity-minus (resistant) breast cancer patients who were ERBB2-positive.

FIG. 15 is a line graph showing the KM estimates for placebo and paclitaxel treatment of sensitivity-positive (sensitive) and sensitivity-minus (resistant) breast tumor patients who were ERBB-2 positive.

FIG. 16 is a line graph showing the KM estimates for Lapatinib (a 4-anilinoquinazoline kinase inhibitor) and paclitaxel treatment of sensitivity-positive (sensitive) and sensitivity-minus (resistant) breast tumor patients (both ERBB2-positive and ERBB2-negative groups).

FIG. 17 is a line graph showing the KM estimates for placebo and paclitaxel treatment of sensitivity-positive (sensitive) and sensitivity-minus (resistant) breast tumor patients (both ERBB2-positive and ERBB2-negative groups).

FIGS. 18a and 18b are line graphs showing the KM estimates for Lapatinib monotherapy of sensitivity-positive (sensitive) and sensitivity-minus (resistant) breast cancer patients who were ERBB2-positive in EGF20009 trial by using (a) a 6-gene predictor set, (b) a single gene CBX5 predictor.

FIGS. 19a, 19b and 19c are line graphs showing the KM estimates for Lapatinib and paclitaxel treatment in EGF30001 trial: (a) stratification of ERBB2-positive patients by using a 6-gene predictor set, (b) stratification of ERBB2-negative patients by using a 6-gene predictor set, (c) stratification of ERBB2-positive patients by using CBX5 as a single gene predictor.

FIGS. 20a and 20b are line graphs showing the KM estimates for Lapatinib and capecitabine treatment of sensitivity-positive (sensitive) and sensitivity-minus (resistant) breast cancer patients who were ERBB2-positive in EGF 100151 trial.

### DETAILED DESCRIPTION OF THE PREFERRED EMBODIMENT

In some embodiments, methods and systems are provided that use splines to predict the magnitude of response of cells to various treatments and also to classify cancer samples (e.g., into sensitive and resistant classes) in an unsupervised manner. In some embodiments, these methods or systems may be used to predict the efficacy of a treatment for a specific person/patient, cancer type or cell line. Furthermore, a hierarchical modeling scheme may be used to integrate profiles from different types of molecular datasets. Methods and systems disclosed herein may provide a generalizable framework for predictive modeling of complex genetic dependencies of diverse physiological responses.

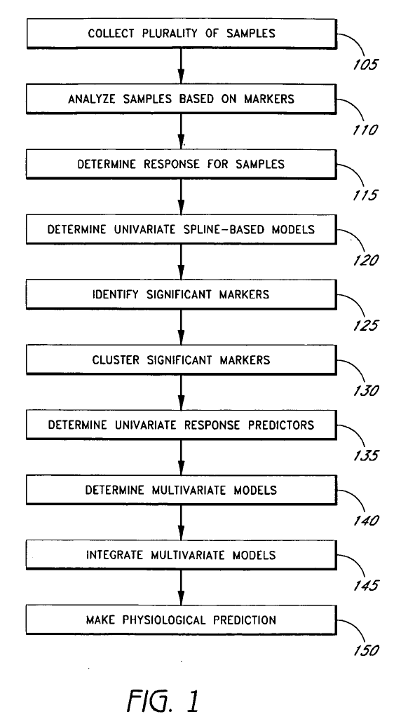

FIG. 1 shows one process 100 for developing a model of a response to a therapeutic treatment. Process 100 beings at step 105 with the collection of a plurality of samples. The samples are obtained from patients and typically comprise a diseased cell or tissue. For example, the sample may comprise a cancer cell or tissue from a tumor. Samples may be collected across a plurality of patients. In some instances, all patients have been diagnosed with a similar or the same disease or condition (e.g., breast cancer), while in other instances, they have not. Control samples may be collected from patients who have not been diagnosed with a disease or condition to be studied or who are otherwise healthy. In some embodiments, the samples comprise a panel of cell lines. This panel may be comprised of cell-lines specific to an organ, e.g. breast cancer cell-lines, pancreatic cancer cell-lines, etc. Alternatively, this panel may comprise of cell-lines from diverse organs, e.g. NCI-60, which includes a panel of sixty cancer cell lines of diverse lineage (lung, renal, colorectal, ovarian, breast, prostate, central nervous system, melanoma and hematological malignancies).

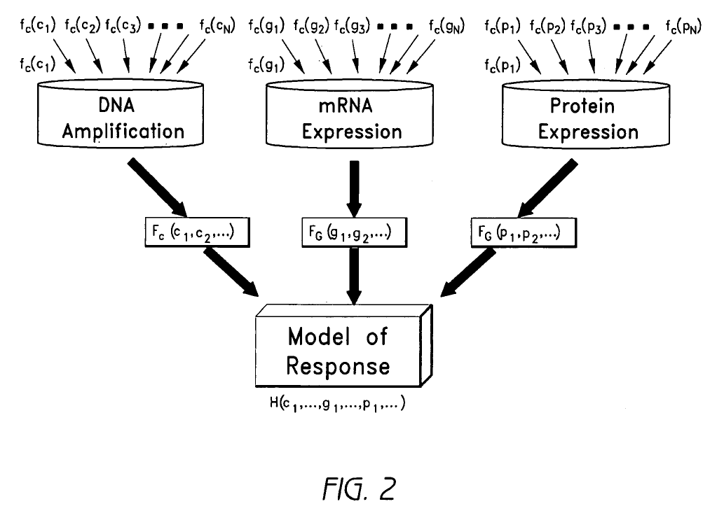

Process 100 continues at step 110 with an analysis of each of the samples based on a plurality of putative markers. The putative markers may comprise different types of marks, such as mRNA expression, protein expression, microRNA expression, CpG methylation, and DNA amplification. In some embodiments, step 110 comprises the determination of molecular profiles of each of the samples. Each of the sampled may be analyzed based on a plurality of putative markers within each type of sample. In some embodiments, the number of putative markers is greater than about 20, 50, 100, 500, 1000, 5000 or 10,000. Notably, the number of molecular predictors (e.g. genes) is typically very large (P˜10⁴) compared to the number of samples available in training sets (typically, N=10-50 for tissue specific cancers). In some embodiments, the ratio of the number of putative markers compared to the number of samples is greater than about 1, 2, 5, 10, 20, 50, 100, 200, 500, or 1000. A quantification value (such as an expression level or amplification value) of each marker (such as an mRNA strand, protein, microRNA, or DNA strand) may be determined for each sample. Techniques and systems to measure expression levels are well known in the art. For example, mRNA levels may be monitored using Affymetrix U133A arrays, and protein levels may be measured using western blot assays. Techniques and systems, such as array-based comparative genomic hybridization technology, to measure DNA amplification are also well known in the art. FIG. 2 shows an example in which N samples are analyzed based on DNA amplification, mRNA expression and protein expression. The amplification of a specific DNA strand, the mRNA expression for a specific mRNA strand, and the protein expression for a specific protein for the ith sample are represented as cᵢ, gᵢ and pᵢ. Notably, while FIG. 2 shows only one c, g and p data set, a number of other c, g and p data sets are typically determined based on DNA, mRNA and proteins. The process need not execute all the steps shown in FIG. 2. For instance, if there is exactly one data set available (e.g. mRNA expression data), only first and second steps may be executed. In some embodiments, only the first step may be executed.

At step 115 of process 100, a physiological response is determined for each of the samples. The physiological response may comprise a binary indication or a magnitude of response. In some embodiments, each sample is contacted with a compound or a drug. The sample may be categorized as being sensitive or resistant (a binary indication) to the compound or drug. In some instances, a quantitative assessment of the effect of the compound or drug on the sample is performed. For example, a GI₅₀ value (a concentration of the compound or drug that causes 50% growth inhibition) or a sensitivity value (equal to the—log(GI₅₀)) may be determined for each sample. Techniques to determine such quantitative assessments are well known in the art. For example, a dose response curve may be generated for each sample using an assay that measures cell viability, such as the CellTiter Glo® Luminescent Cell Viability assay, which may then be used to estimate GI₅₀ for the sample.

Process 100 continues at step 120 with the determination of a plurality of univariate models using spline analysis. Each univariate model may be based on one of the plurality of putative markers. In some embodiments, functions relating the physiological responses to putative markers are fit with splines. A spline is defined as a piecewise polynomial function separated at point called knots. In some embodiments, the spline comprises a linear spline, wherein the spline has a degree of one. Linear splines are linear above a knot, and zero below it. Additionally, linear splines provide a complete set of basis functions, and thus, can facilitate comprehensive modeling of the response profiles. Fitting with splines may include identification of optimal partitions and fitting a function (e.g., a linear function) within each partition. The partition may, in effect, separate samples based on their class identity. The dependence of the physiological response on the putative marker may vary between the classes, but since the fitted function is continuous, this difference may thereby be determined (learnt) in a single optimization determination. In FIG. 2, univariate functions f_{c}(cᵢ), f_{g}(gᵢ) and fₚ(pᵢ) are determined based on physiological responses and the DNA amplification data cᵢ, mRNA expression data gᵢ, or protein expression data pᵢ, respectively.

The spline may comprise an adaptive spline. The adaptive splines can simultaneously account for class information and magnitude of response within a single framework. As described in more detail below, the spline analysis may provide superior fitting and/or better predictions as compared to supervised classification or linear regression analyses. An adaptive spline comprises at least one un-fixed knot. That is, the position of the knot is determined based on (e.g., fit to) the data. Adaptive splines can provide a flexible framework to model a variety of responses ranging from bimodal distributions to more continuous distributions. If the spline model has no knots, then it is a linear model. If the model has one knot and the slope of the line is zero in one partition, then the model is equivalent to a single linear spline. If the model has two knots and the slopes of the lines are zero in two exterior partitions (but non-zero slope in the interior partition), then it is the same as a classification model. An adaptive spline model containing M internal knots, ξ₁, . . . ξ_{M}, is written as (ξ₀ and ξ_{M+1} are the boundary values of x):

$$log ( {GI}_{50} ) = {a}_{0} + {\sum }_{k = 1}^{M + 1} {a}_{k} {h}_{k} ( x ) ≡ f ( x ) , \qquad ( 1 )$$

where x represents the appropriate predictor variable: logarithm of expression (mRNA or protein) or DNA amplification. α₀ is the intercept and αₖ's are the slopes. The function hₖ(x) is defined as:

hₖ(x)=(x−ξₖ₋₁)₊−(x−ξₖ)₊ (2)

The linear spline (x−ξ)₊ is defined as: (x−ξ)₃₀ =x−ξ, for x>ξ, and 0, otherwise. For a fixed number of knots, the algorithm enumeratively searches for the best location of knots. Model parameters may then be estimated by minimizing the residual sum of squares. In some embodiments, the spline comprises a non-adaptive spline, in which the position of the knot/s are fixed and do not depend on the data. The spline may also be partially adaptive, such that the positions of one or more knots are fixed while the positions of one or more other knots are not fixed, or such that the positions of one or more knots are constrained.

In one instance, the response data may be modeled as sum of linear splines, where the predictor variables are markers such as DNA amplification, mRNA expression or protein expression levels. The adaptive splines model containing M internal knots, ξ₁, . . . ξ_{M}, is written as (ξ₀ and ξ_{M+1} are the boundary values of x):

$$log ( {GI}_{50} ) = {a}_{0} + {\sum }_{k = 1}^{M + 1} {a}_{k} {h}_{k} ( x ) ≡ f ( x ) , \qquad ( 3 )$$

where x represents the appropriate predictor variable. α₀ is the intercept and αₖ's are the slopes. The function hₖ (x) is defined as:

hₖ(x)=(x−ξₖ₋₁)₊−(x−ξₖ)₊ (4)

The linear spline (x−ξ)₊ is defined as: (x−ξ)₊=x−ξ, for x >ξ, and 0, otherwise. The optimization in equation (4) becomes much easier if f(x) is rewritten in terms of the values, {gₖ}, achieved by the spline f(x) at the knots {ξₖ}:

$$f ( x ) = {g}_{0} ( 1 - {\hat{h}}_{1} ) + {\sum }_{j = 1}^{M} {g}_{j} ( {\hat{h}}_{j} - {\hat{h}}_{j + 1} ) + {g}_{M + 1} {\hat{h}}_{M + 1} , \qquad ( 5 )$$

where ĥₖ≡ĥₖ(x) is defined as:

$${\hat{h}}_{k} ( x ) = \frac{{h}_{k} ( x )}{{ξ}_{k} - {ξ}_{k - 1}} \qquad ( 6 a )$$

and the coefficients {αₖ} are related to the functional values of the spline, {gₖ}, as follows:

$$\begin{matrix}{a}_{0} = {g}_{0} , & ( 6 b ) \\ {a}_{k} = \frac{{g}_{k} - {g}_{k - 1}}{{ξ}_{k} - {ξ}_{k - 1}} , for k = 1 , K ( M + 1 ) & ( 7 c )\end{matrix}$$

Minimization of residual sum of squares allows one to compute the functional values, (g₀, g₁, K, g_{M+1})≡ [symbol] , as follows:

[symbol]

=A⁻¹b, (7)

where the entries of A, a symmetric tridiagonal matrix, and the vector [symbol] are calculated as follows:

$$\begin{matrix}\begin{matrix}{A}_{k , k - 1} = {A}_{k - 1 , k} \\ = \frac{1}{N} {\sum }_{i = 1}^{N} [ {\hat{h}}_{k} ( {x}_{i} ) - {\hat{h}}_{k + 1} ( {x}_{i} ) ] [ {\hat{h}}_{k - 1} ( {x}_{i} ) - {\hat{h}}_{k} ( {x}_{i} ) ] ,\end{matrix} \\ k = 1 , K , ( M + 1 ) & ( 8 a ) \\ {A}_{k , k} = \frac{1}{N} {\sum }_{i = 1}^{N} {[ {\hat{h}}_{k} ( {x}_{i} ) - {\hat{h}}_{k + 1} ( {x}_{i} ) ]}^{2} , \\ k = 1 , K , M & ( 8 b ) \\ {b}_{k} = \frac{1}{N} {\sum }_{i = 1}^{N} {y}_{i} [ {\hat{h}}_{k} ( {x}_{i} ) - {\hat{h}}_{k + 1} ( {x}_{i} ) ] , \\ k = 1 , K , M & ( 8 c ) \\ {\hat{h}}_{0} ≡ 1 , {\hat{h}}_{M + 2} ≡ 0 & ( 8 d )\end{matrix}$$

Here, y=log(GI₅₀) and the running variable i refers to the cell-lines (total count=N). The first and last diagonal elements of A, and first and last elements of [symbol] are computed as:

$$\begin{matrix}{A}_{00} = \frac{1}{N} {\sum }_{i = 1}^{N} {[ 1 - {\hat{h}}_{1} ( {x}_{i} ) ]}^{2} & ( 8 e ) \\ {A}_{M + 1 , M + 1} = \frac{1}{N} {\sum }_{i = 1}^{N} {\hat{h}}_{M + 1}^{2} & ( 8 f ) \\ {b}_{0} = \frac{1}{N} {\sum }_{i = 1}^{N} {y}_{i} [ 1 - {\hat{h}}_{1} ( {x}_{i} ) ] & ( 8 g ) \\ {b}_{M + 1} = \frac{1}{N} {\sum }_{i = 1}^{N} {y}_{i} {\hat{h}}_{M + 1} ( {x}_{i} ) & ( 8 h )\end{matrix}$$

Matrix inversion of the tridiagonal matrix A leads to the vector [symbol] in equation (7).

In one embodiment, each univariate model comprises a sum of linear splines, where the predictor variable is the specific molecular profile of the potential marker. For a fixed number of knots, which define the partitions, an algorithm may identify location of knots by, for example, minimizing the residual sum of squares. In some embodiments, the number of knots is predetermined, while in other embodiments, the number of knots is determined based on the data. In one instance, a leave-one-out cross-validation method (LOOCV) is used to determine the number of knots.

Process 100 continues at step 125 with the identification of significant markers based on the univariate models. In some embodiments, significant markers are identified based on how well the spline could fit a function relating the physiological response to the marker. For example, a p-value may be used to determine significant markers. In some embodiments, LOOCV error of the spline fit is used to determine whether the marker is significant. A value associated with the fit (e.g., a p-value or LOOCV error) may be compared to a fixed and/or relative threshold.

At step 130 of process 100, the significant markers are clustered. The markers may be clustered by an unsupervised or a supervised process. The clustering may comprise hierarchical clustering. In some embodiments, the number of clusters is predetermined, while in others it is not. For example, it may be determined that the markers will be clustered into one resistant class and one sensitive class. Identification characteristics of the classes may be determined before or after the clustering. For example, the markers may be clustered into a resistant and sensitive class, or the markers may be clustered into two classes, which are later determined to correspond to resistant and sensitive classes.

At step 135 of process 100, univariate response predictors are determined. Each univariate model can be used to make a single prediction of the physiological response of a biological sample not used in the generation of the univariate model. For example, after a univariate model has been determined, the univariate model may be used to predict cell growth inhibition or apoptosis based on the expression of a specific protein. Thus, the predictor of cell viability or apoptosis of a new sample may be predicted based on the protein expression in the cells of the sample. In some embodiments, univariate predictors are determined for all putative markers. In other embodiments, univariate predictors are determined for significant markers. Thus, there may be a set of predictors, each predictor associated with a different marker (and thus with a different univariate model).

At this step, one may choose to evaluate the biological relevance of the statistically important molecular markers. This can be done, for example, by examining which Gene Ontology terms belonging to biological process or molecular function or cellular component category are enriched in this marker set. One may choose to use a different database, for instance, a commercially available database of biochemical functions, pathways and analogously defined entities. One such example, though not limiting, is the Ingenuity database (http ://www.ingenuity.com/).

Process 100 continues at step 140 with the formation of a multivariate model for each type of marker (e.g., mRNA expression, protein expression, microRNA expression, CpG methylation, or DNA amplification). The multivariate model may be formed by combining univariate predictors. In some embodiments, the multivariate model comprises weighted averages of the univariate models. All univariate predictors, all significant univariate predictors or a subset of the univariate predictors may be used in developing the multivariate model. The weights in the weighted voting scheme may be determined based on a characteristic of a fit, such as a correlative fit or a spline fit, used to obtain the univariate model. For example, the weight associated with each univariate predictor may be proportional to a magnitude of a correlation between the physiological response and the corresponding marker. The weight may be associated with a coefficient or significance of a spline fit used to obtain the univariate model. In some embodiments, the weights may be proportional to the logarithm of the p-value of the univariate spline model. In FIG. 2, multivariate models F_{C}, F_{G}, and Fₚ are determined based on the corresponding univariate models for each of DNA amplification, mRNA expression and protein expression, respectively.

One example of a multivariate model using weighted voting is:

$${log ( {GI}_{50} )}_{D} = {\sum }_{g = 1}^{{N}_{G}} {w}_{g}^{D} * log ( {( {GI}_{50} )}_{D}^{g} , \qquad ( 9 )$$

where D indicates a data-type, g indicates a prioritized univariate predictor for this data-type, log(GI₅₀)_{D}^{g} is the predicted value of log(GI₅₀) based on the feature g, N_{G} the total number of predictors used, and w^{D}_{g} indicates the normalized weight for this univariate feature for data type D, being proportional to the magnitude of correlation with response:

$${w}_{g}^{D} = log ( {p}_{g}^{D} ) / {\sum }_{g = 1}^{{N}_{G}} log ( {p}_{g}^{D} ) \qquad ( 10 )$$

where p^{D}_{g} is the p-value of the univariate feature g for data type D. The model size, N_{G}, may be determined by minimizing the LOOCV error.

In some embodiments, a multivariate model comprises a fit based on the significant feature variables. This fit may be independent from equations, variables and/or fits of the univariate models. In some embodiments, the fit includes some parameters from the univariate models but learns other parameters based on the data. In one example, knots of splines from the univariate models are used, but polynomial equations used in the splines are learned based on the data. In another example, once significant markers are identified, a spline equation may be used to identify a new multivariate relationship between the physiological response and the significant markers. For example, once significant markers are identified, a spline equation may be used to identify a new multivariate relationship between the physiological response and the significant markers. A fit used in determination of a multivariate model may be based on any appropriate fitting technique, such as a least squares fitting technique.

Process 100 continues at step 145 with the integration of the multivariate models across marker types. One example of an integrated model across data types is:

$$log ( {GI}_{50} ) = {\sum }_{D = 1}^{{N}_{M}} {W}_{D} * {log ( {GI}_{50} )}_{D} , \qquad ( 11 )$$

where N_{M}=total number of data-types. The normalized weight W_{D} is proportional to the average log of p-values, and is calculated as:

$${W}_{D} = {w}_{D}^{avg} / {\sum }_{D = 1}^{{N}_{M}} {w}_{D}^{avg} , \qquad ( 12 )$$

where w_{D}^{avg} is the average log (p-value) of the univariate predictors included in the model for this data type D.

In FIG. 2, the model H predicts a response based on DNA amplification, mRNA expression and protein expression for a sample. The model is obtained by integrating the multivariate models F_{C}, F_{G}, and Fₚ.

At step 150 of process 100, a physiological prediction is made using a model described herein. The physiological prediction may include a prediction as to the response (e.g., the same as or similar to the response determined in step 115) of a new biological sample (e.g., cell type, cancer or an alive or deceased patient). Quantification values (e.g., expression, concentration, or amplification) of specific, significant or all markers in the sample may be determined. In a first example, the samples collected in step 105 were breast cancer cell-lines, and the response determined in step 115 was cell viability in response to a drug. Quantification values from a new sample collected from another cell-line or a patient diagnosed with breast cancer may then be determined and the cell viability response to the drug may be predicted using the model. In a second example, the samples collected in step 105 may be collected from patients diagnosed with a plurality of cancer types, and the response determined in step 115 was cell viability in response a treatment. Quantification values from a new sample may then be collected from another patient diagnosed with cancer (of a new type or of one the plurality of types) and the cell viability response to the treatment may be predicted using the model.

The physiological prediction may include a classification. In one instance, a new sample may be determined to be resistant or sensitive to a treatment. For example, if the sample comprises expression of certain markers below identified knots in spline equations, the sample may be determined to be resistant to a treatment. In another instance, a classification is predicted for a sample of the samples collected in step 105. For example, a specific cell line may be classified as resistant to a treatment.

The physiological prediction may include a prediction related to a patient. For example, the physiological prediction may estimate survival time, likelihood of survival, or probability of survival within a time period. The prediction may be related to the probability of experiencing an adverse event or an interaction of treatments.

The physiological prediction may include a prediction related to treatment efficacy. In some embodiments, a testing sample is obtained from a person who is or may be suffering from a specific disease. Quantification values of the testing sample are determined, and a physiological response is predicted based on a model described herein. This prediction may be used to predict how effective a treatment would be for the person who provided the testing sample. In other embodiments, the testing sample is obtained from a specific cell line or from a patient suffering from a specific disease, and the predicted physiological response may then be used to predict how effective a treatment would be for the cell line or against the specific disease. The physiological prediction may include an efficacy value. For example, it may be predicted that a treatment may be effective in eliminating 50% of a specific tumor (e.g., for a specific person). As another example, it may be predicted that there is a 60% probability that a treatment will eliminate a specific tumor type (e.g., for a specific person). The physiological prediction related to treatment efficacy may comprise a value associated with cell viability and/or apoptosis or survival, or even related to metabolism, e.g. glycolytic index value. In some embodiments, the prediction may comprise a binary result, e.g. sensitive or resistant to a drug.

The physiological prediction may include a risk probability assessment or a diagnosis. For example, the samples collected in step 105 may be collected from subjects suffering from a disease and healthy subjects or from subjects suffering from multiple strains of a disease. A spline-based method may naturally separate samples from the two groups. Thus, analysis of specific quantification values in a new sample may indicate whether a patient suffers from a specific disease.

The physiological prediction may include identification of specific markers. The specific markers may include significant markers and/or those determined to be indicative of a disease, a classification (e.g., of a cell, tumor or cancer), or a treatment response.

The physiological prediction may include a treatment. The treatment may be one that is predicted to be effective in treating a disease or condition. In one instance, a plurality of models is determined, each relating a response to a different treatment to quantification values. By determining quantification values in a new sample, a single treatment among the different treatments may be identified as being most probable to be effective. The treatment may be one previously used in determining responses of the samples in step 115 or may be a new treatment. For example, based on one or more models, properties of treatments indicative of efficacy may be identified and effective treatments may be predicted.

The physiological prediction may include a number, a percent, a classification, or a description. For example, the prediction may include a cell viability number predicted to occur in response to a treatment. The prediction may include a percent (e.g., of cell viability) predicted to occur in response to a treatment relative to no treatment. The prediction may include a number indicating a predicted response relative to responses or predicted responses of other samples. The prediction may include a discrete response, such as binary or trinary responses. In one such example, the prediction may be either resistant or sensitive. The prediction may include confidence intervals.

In some embodiments, a computer-readable medium or computer software comprises instructions to perform one or more steps of process 100 (e.g., steps 120-150). The software may comprise instructions to output (e.g., display, print or store) the physiological prediction.

In some embodiments, one or more steps shown in FIG. 1 are not included in process 100. For example, step 130 may be excluded from process 100. In some embodiments, additional steps are included in process 100. In some embodiments, the steps are arranged differently than shown in FIG. 1. Multiple steps may be combined (e.g., steps 125 and 135 may be combined into one step), and/or single steps may be separated into a plurality of steps.

The hierarchical component of process 100 allows the integration of profiles from diverse molecular datasets. Additionally, while other analyses use only a subset of the samples for predicting physiological response, process 100 accounts for responses from all samples, thereby leading to nonlinear response signatures and facilitating tissue-specific analysis. A subset of samples may also be used in the process 100,

Process 100 provides a number of advantages over supervised classification, in which samples are segregated into sensitive and resistant classes based on training data, as process 100 provides a quantitative value predicted for the physiological response. This magnitude can provide useful information, which is often lost upon discretizing the data into various classes. In some embodiments, fewer markers are needed to predict physiological responses as compared to other methods. For example, fewer markers may be needed in models described herein as compared to models that do not account for response magnitude but instead rely on classification. Fewer markers also make their clinical deployment very cost-effective.

Furthermore, in supervised classification methods, one needs to select at least one response threshold to label samples in training set with their different class-types, e.g. sensitive versus resistant for drug response. However, since this threshold is not known in advance, there is substantial amount of subjectivity in the analysis. An alternative strategy is to use samples that are at the extremes of sensitivity and resistance to train the model, but then a substantial fraction of the data remains unused. This poses a significant problem for analysis of organ-specific cancer datasets, as such data sets are often quite small in size. Finally, cancer cells often exhibit complex response patterns. For instance, samples can segregate into groups, characteristic of distinct classes, while displaying significant variation in magnitude within classes.

Moreover, spline-based methods described herein can be applied to smaller datasets than other methods (e.g., those that exclude data from the training set), as the spline-based methods can accurately model all data points together, i.e. without filtering out any sample. For example, these methods may be used to study responses of specific tumor types.

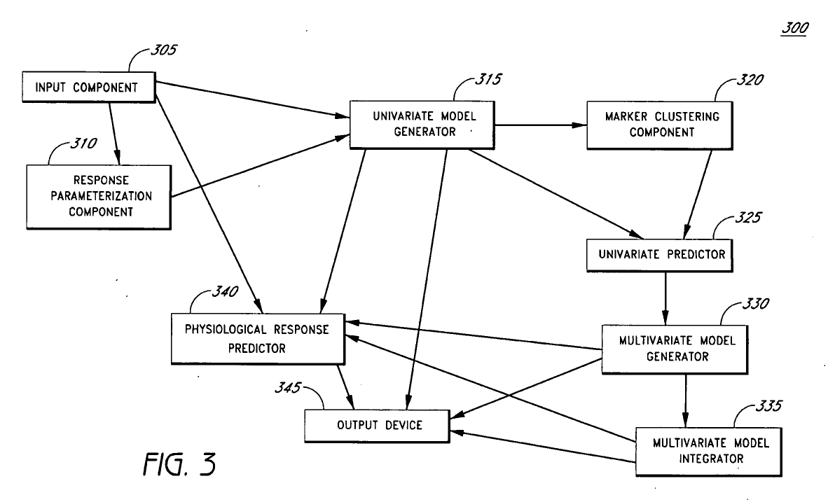

In some embodiments, a system 300 (e.g., a computer system) is provided to make a physiological prediction about a treatment response. As shown in FIG. 3, the system may comprise an input component 305. The input component may comprise any input device such as a keyboard, a mouse, or a memory storage device (e.g., a disk, a compact disc, a DVD, or a USB drive). The input component may be configured to receive data related to physiological responses (e.g., to one or more treatments) of a plurality of samples. The input component 305 may be configured to receive data related to quantification values of a plurality of samples. In one example, a user inputs mRNA expression values, DNA amplification values, microRNA expression values, CpG methylation values, protein expression values for each of a plurality of samples using a keyboard. The user may also input cell viability value/s associated with a treatment (e.g., for a plurality of drug concentrations). The input component 305 may be configured to receive data related to training samples and/or to test samples.

The system 300 may comprise a response parameterization component 310. The response parameterization component 310 determines the efficacy of a treatment for each sample (e.g., each training sample) based on data input at the input component 305, such as a plurality of cell viability or apoptosis values. For example, the GI₅₀ may be determined based on cell viability values associated with different drug concentrations. In some instances, the system 300 does not include a response parameterization component 310. For example, the component 310 may not be included if the user may input a GI₅₀ value at the input component 305.

The system 300 may comprise a univariate model generator 315. The univariate model generator 315 determines of a plurality of univariate models using spline analysis, the univariate model being any univariate model as described herein. The univariate model generator 315 determines the univariate models based on the data input at input component 305 and optionally the efficacy values from efficacy determination component 310. Each univariate model may predict a value of a physiological response (e.g., the physiological response that was input at the input component 305) based on a single marker (e.g., one of the markers that was input at the input component 305).

The system 300 may comprise a marker clustering component 320. The marker clustering component 320 may cluster markers input at input component 305 by unsupervised, hierarchical clustering or any other process as described herein. The marker clustering component 320 may or may not use univariate models from univariate model generator 315.

The system 300 may comprise a univariate predictor 325. The univariate predictor 325 may determine univariate response predictions based on univariate models from the univariate model generator 315 and/or based on the marker clusters from marker clustering component 320 by a process described herein. For example, each univariate models associated with a plurality of markers can be used to make a single prediction of the physiological response of a sample not used in the generation of the univariate models.

The system 300 may comprise a multivariate model generator 330. The multivariate model generator 330 may determine a multivariate model as described herein. For example, the multivariate model may be formed by combining univariate predictions from the univariate predictor 325 using weighted averages of the univariate response predictions.

The system 300 may comprise a multivariate model integrator 335. The multivariate model integrator 335 may integrate multivariate models from the multivariate model generator 330 by a process described herein.

The system 300 may comprise a physiological response predictor 340. The physiological response predictor 340 may determine a physiological prediction as described herein by a process as described herein. For example, the physiological response predictor 340 may predict a cell viability of a new sample based on an integrated model from the multivariate model integrator 355.

The system 300 may comprise an output device 345. The output device may comprise any appropriate output device, such as a display screen or a printer. The output device may be configured to store output onto a data storage medium. The output device may output models or model components (e.g., coefficient, significance, or fit values), such as those from one or more univariate models generated by univariate model generator 315, one or more multivariate models generated by multivariate model generator 330, or one or more integrated models generated by the multivariate model integrator 335. The output device may output a physiological prediction determined by the physiological predictor 340.

In some embodiments, one or more components or connections shown in FIG. 3 are not included in system 300. In some embodiments, additional components or connections are included in system 300. In some embodiments, the components are connected differently than shown in FIG. 3.

The system 300 may comprise a memory. The system 300 may be connected to a network, such as the internet. The system 300 may comprise a computer system including a CPU and a memory such as the ROM. Such memory medium may store a program or software for executing steps of process 100. The memory medium can be composed of a semiconductor memory such as a ROM or a RAM, or an optical disk, a magnetooptical disk or a magnetic medium. It may also be composed of a CD-ROM, a floppy disk, a magnetic tape, a magnetic card or a non-volatile memory card.

As used herein, an increased or decreased expression level is an expression level of a gene that is more than or less than, respectively, the expression level of the same gene in a normal tissue or cell sample. For example, the normal cell or tissue may be a cell or tissue sample of non-cancerous cells from a patient or another person that does not have cancer. In some embodiments, an increased or decreased expression level is an expression level of a gene that is more than or less than, respectively, the average expression level of the same gene in a panel of normal cell lines or cancer cell lines. In some embodiments, an increased or decreased expression level is an expression level that is relatively more than or less than, respectively, the expression of a housekeeping gene, such as a gene encoding GAPDH. In some embodiments, a high or low expression level of a gene is a value equal to or higher or lower, respectively, than the average value (log₂(expression)) described for the corresponding gene in Table 10.

Techniques and systems to measure expression levels are well known by persons skilled in the art. For example, quantitative mRNA levels of the transcripts may be monitored using a quantitative PCR-analysis with primer combinations to amplify said gene specific sequences from cDNA obtained by reverse transcription of RNA extracted from a sample obtained from a subject. These techniques are known to persons skilled in the art (see Sambrook and Russell, Molecular Cloning: A Laboratory Manual, Cold Spring Harbor Laboratory Press, Cold Spring Harbor, N.Y., 2001; Schena M., Microarray Biochip Technology, Eaton Publishing, Natick, Mass., 2000). It might further be preferred to measure transcription products by chip-based microarray technologies, including the branch capture (BC) assay from Panomics and Affymetrix U133A arrays.

Protein levels may be detected using an immunoassay, an activity assay, and/or a binding assay. These assays can measure the amount of binding between a protein molecule of interest and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g., ELISAs, Western blot and other techniques known to persons skilled in the art (see Harlow and Lane, Antibodies: A Laboratory Manual, Cold Spring Harbor Laboratory Press, Cold Spring Harbor, N.Y., 1999 and Edwards R. Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

DNA amplification may be detected using Southern blot assay, quantitative PCR, immunohistochemistry (IHC), fluorescent in situ hybridization (FISH), or an array-based comparative genomic hybridization technology. These techniques are known to persons skilled in the art (see Sambrook and Russell, Molecular Cloning: A Laboratory Manual, Cold Spring Harbor Laboratory Press, Cold Spring Harbor, N.Y., 2001).

In one embodiment, a cancer patient is either a patient who is known to be ERBB2-positive, that is, a patient overexpresses the ERBB2 protein, or a patient who is not known whether he or she is ERBB2-positive or not. When the patient is not known whether to be ERBB2-positive or not, the ERBB2 status of the patient is to be determined.

To determine whether a patient is an ERBB2-positive patient, the expression level of a gene encoding ERBB2 in a patient is measured. Methods for measuring the expression level of a gene encoding ERBB2 are well known to those skilled in the art. Methods of assaying for ERBB2 or HER2 protein overexpression include methods that utilize immunohistochemistry (IHC) and methods that utilize fluorescence in situ hybridization (FISH). A commercially available IHC test is PathVysion® (Vysis Inc., Downers Grove, Ill.). A commercially available FISH test is DAKO HercepTest® (DAKO Corp., Carpinteria, Calif.). The expression level of a gene encoding ERBB2 can be measured using an oligonucleotide derived from the nucleotide sequence of SEQ ID NO: 1, 7, or 26.

In some embodiments, a method for identifying a cancer patient suitable for treatment with a 4-anilinoquinazoline kinase inhibitor is provided, the method comprising: (a) detecting the expression level of one or more genes described in Table 7a in a sample from the patient, and (b) comparing the expression level of the same gene(s) from the patient with the expression level of the gene(s) in a normal tissue sample or a reference expression level (such as the average expression level of the gene(s) in a cell line panel, a cancer cell, a tumor panel, or the like). An increase in the expression level of GRB7, or a decrease in the expression level of CRK, ACOT9, CBX5, or DDX5 indicates the patient is suitable for treatment with the 4-anilinoquinazoline kinase inhibitor. In addition, a decrease in the expression level of GRB7, or an increase in the expression level of CRK, ACOT9, CBX5, or DDX5 indicates the patient is resistant to treatment with the 4-anilinoquinazoline kinase inhibitor.

In some embodiments, a method for identifying a cancer patient suitable for treatment with a 4-anilinoquinazoline kinase inhibitor is provided, the method comprising: (a) detecting the expression level of CBX5 in a sample from the patient, and (b) comparing the expression level of CBX5 from the patient with the expression level of CBX5 in a normal tissue sample or a reference expression level (such as the average expression level of CBX5 gene in a cell line panel, a cancer cell, a tumor panel, or the like). A decrease in the expression level of CBX5 indicates the patient is suitable for treatment with the 4-anilinoquinazoline kinase inhibitor. In addition, an increase in the expression level of CBX5 indicates the patient is resistant to treatment with the 4-anilinoquinazoline kinase inhibitor.

In some embodiments, a method for identifying a cancer patient suitable for treatment with a 4-anilinoquinazoline kinase inhibitor is provided, the method comprising: (a) detecting the expression level of one or more genes described in Table 7b in a sample from the patient, and (b) comparing the expression level of said gene(s) from the patient with the expression level of said gene(s) in a normal tissue sample or a reference expression level (such as the average expression level of the gene in a cell line panel, a cancer cell, a tumor panel, or the like). An increase in the expression level of AK3L1, DDR1, CP, CLDN7, GNAS, SERPINB5, DGKZ, TRIM29, GABARAPL1, and SORL1, or a decrease in the expression level of NOLC1, FLJ10357, or WDR19 indicates the patient is suitable for treatment with the 4-anilinoquinazoline kinase inhibitor. In addition, a decrease in the expression level of AK3L1, DDR1, CP, CLDN7, GNAS, SERPINB5, DGKZ, TRIM29, GABARAPL1, and SORL1, or an increase in the expression level of NOLC1, FLJ10357, or WDR19 indicates the patient is resistant to treatment with the 4-anilinoquinazoline kinase inhibitor.

In some embodiments, a method for identifying a cancer patient suitable for treatment with a 4-anilinoquinazoline kinase inhibitor is provided, the method comprising: (a) detecting the expression level of one or more genes described in Tables 7a and 7b in a sample from the patient, and (b) comparing the expression level of said gene(s) from the patient with the expression level of said gene(s) in a normal tissue sample or a reference expression level (such as the average expression level of said gene(s) in a cell line panel or a cancer cell or tumor panel, or the like). An increase in the expression level of GRB7, AK3L1, DDR1, CP, CLDN7, GNAS, SERPINB5, DGKZ, TRIM29, GABARAPL1, and SORL1, or a decrease in the expression level of CRK, ACOT9, CBX5, DDX5, NOLC1, FLJ10357, or WDR19 indicates the patient is suitable for treatment with the 4-anilinoquinazoline kinase inhibitor. A decrease in the expression level of GRB7, AK3L1, DDR1, CP, CLDN7, GNAS, SERPINB5, DGKZ, TRIM29, GABARAPL1, and SORL1, or an increase in the expression level of CRK, ACOT9, CBX5, DDX5, NOLC1, FLJ10357, or WDR19 indicates the patient is resistance to treatment with the 4-anilinoquinazoline kinase inhibitor.

The GRB7 protein is also known as growth factor receptor-bound protein 7. The expression level of a gene encoding GRB7 can be measured using an oligonucleotide derived from the nucleotide sequence of SEQ ID NO:2, 8, or 27.

The CRK protein is also known to be encoded by cDNA FLJ38130 fis, clone D6OST2000464. The expression level of a gene encoding CRK can be measured using an oligonucleotide derived from the nucleotide sequence of SEQ ID NO:3, 9, or 28.

The ACOT9 protein is also known as acyl-CoA thioesterase 9. The expression level of a gene encoding ACOT9 can be measured using an oligonucleotide derived from the nucleotide sequence of SEQ ID NO:4, 10, or 29.

The FLJ31079 protein is also known to be encoded by cDNA clone IMAGE:4842353. The FLJ31079 protein is now annotated as CBX5 protein (heterochromatin protein 1-alpha). The expression level of a gene encoding FLJ31079 (CBX5) can be measured using an oligonucleotide derived from the nucleotide sequence of SEQ ID NO:5, 11, or 30.

The DDX5 protein is also known as DEAD (Asp-Glu-Ala-Asp) box polypeptide 5. The expression level of a gene encoding DDX5 can be measured using an oligonucleotide derived from the nucleotide sequence of SEQ ID NO:6, 12, or 31.

The AK3L1 is also known as adenylate kinase 3-like 1. The expression level of a gene encoding AK3L1 can be measured using an oligonucleotide derived from the nucleotide sequence of SEQ ID NO:13 or 32.

The DDR1 is also known as discoidin domain receptor family, member 1. The expression level of a gene encoding DDR1 can be measured using an oligonucleotide derived from the nucleotide sequence of SEQ ID NO:14 or 33.

The CP is also known as ceruloplasmin (ferroxidase). The expression level of a gene encoding CP can be measured using an oligonucleotide derived from the nucleotide sequence of SEQ ID NO:15 or 34.

The CLDN7 is also known as claudin 7. The expression level of a gene encoding CLDN7 can be measured using an oligonucleotide derived from the nucleotide sequence of SEQ ID NO:16 or 35.

The GNAS is also known as GNAS complex locus. The expression level of a gene encoding GNAS can be measured using an oligonucleotide derived from the nucleotide sequence of SEQ ID NO:17 or 36.

The SERPINB5 is also known as serpin peptidase inhibitor, clade B (ovalbumin), member 5. The expression level of a gene encoding SERPTNB5 can be measured using an oligonucleotide derived from the nucleotide sequence of SEQ ID NO:18 or 37.

The DGKZ is also known as diacylglycerol kinase, zeta 104 kDa. The expression level of a gene encoding DGKZ can be measured using an oligonucleotide derived from the nucleotide sequence of SEQ ID NO:19, or 38.

The NOLC1 is also known as nucleolar and coiled-body phosphoprotein 1. The expression level of a gene encoding NOLC1 can be measured using an oligonucleotide derived from the nucleotide sequence of SEQ ID NO:20 or 39.

The TRIM29 is also known as tripartite motif-containing 29. The expression level of a gene encoding TRIM29 can be measured using an oligonucleotide derived from the nucleotide sequence of SEQ ID NO:21 or 40.

The GABARAPL1 is also known as GABA(A) receptor-associated protein like 1 /// GABA(A) receptors associated protein like 3. The expression level of a gene encoding GABARAPL1 can be measured using an oligonucleotide derived from the nucleotide sequence of SEQ ID NO:22 or 41.

The FLJ10357 is also known to be encoded by cDNA clone IMAGE:3506356. The expression level of a gene encoding FLJ10357 can be measured using an oligonucleotide derived from the nucleotide sequence of SEQ ID NO:23 or 42.

The WDR19 is also known as WD repeat domain 19. The expression level of a gene encoding WDR19 can be measured using an oligonucleotide derived from the nucleotide sequence of SEQ ID NO:24 or 43.

The SORL1 is also known as sortinlin-related receptor, L (DLR class) A repeats-containing. The expression level of a gene encoding SORL1 can be measured using an oligonucleotide derived from the nucleotide sequence of SEQ ID NO:25 or 44.

In some embodiments, the nucleotide sequence of a suitable fragment of the gene is used, or an oligonucleotide derived thereof The length of the oligonucleotide is of any suitable length. A suitable length can be at least 10 nucleotides, 20 nucleotides, 30 nucleotides, 50 nucleotides, 100 nucleotides, 200 nucleotides, or 400 nucleotides, and up to 500 nucleotides or 700 nucleotides. A suitable nucleotide is one which binds specifically to a nucleic acid encoding the target gene.

Compounds and formulations of 4-anilinoquinazoline kinase inhibitors suitable for use in the present invention, and the dosages and methods of administration thereof, are taught in U.S. Pat. Nos. 6,391,874; 6,713,485; 6,727,256; 6,828,320; and 7,157,466, and International Patent Application Nos. PCT/EP97/03672, PCT/EP99/00048, and PCT/US01/20706 (which are incorporated in their entireties by reference). In some embodiments, the 4-anilinoquinazoline kinase inhibitor is Lapatinib. In some embodiments, the Lapatinib is Lapatinib ditosylate monohydrate, which is commercially available under the brand name TYKERB® (GlaxoSmithKline; Research Triangle Park, NC). The prescription information of TYKERB® (Full Prescribing Information, revised March 2007, GlaxoSmithKline), which is incorporated in its entirety by reference, teaches one method of administration of Lapatinib to a patient.

In some embodiments, a method of treating a cancer patient is provided. The method comprising: (a) identifying a cancer patient who is suitable for treatment with a 4-anilinoquinazoline kinase inhibitor, and (b) administering a therapeutically effective amount of the 4-anilinoquinazoline kinase inhibitor to the cancer patient. The term “therapeutically effective amount” as used herein refers to the amount of a 4-anilinoquinazoline kinase inhibitor that is sufficient to prevent, alleviate or ameliorate symptoms of cancer or to prolong the survival of the patient being treated. Determination of a therapeutically effective amount is within the capability of those skilled in the art. In some embodiments, the therapeutically effective amount is the amount effective to at least slow the rate of tumor growth, slow or arrest the progression of cancer, or decrease tumor size. Tumor growth and tumor size can be measured using routine methods known to those skilled in the art, including, for example, magnetic resonance imaging and the like. In some embodiments, the cancer is breast cancer and the cancer patient is a breast cancer patient. In some embodiments, the breast cancer patient is an ERBB2-positive breast cancer patient. In some embodiments, a “therapeutically effective amount” of a 4-anilinoquinazoline kinase inhibitor is an amount effective to result in a downgrading of a breast cancer tumor, or an amount effective to slow or prevent the progression of a breast cancer tumor to a higher grade.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

### EXAMPLES

### Example 1

### Fitting Using Linear Splines

The suitability of using linear splines as basis functions was tested using simulated datasets. Class-like structure of underlying response data has often been assumed while performing the analyses. The simulations helped us to evaluate the potential of different approaches in the context of this assumption for such small N. large P problems.

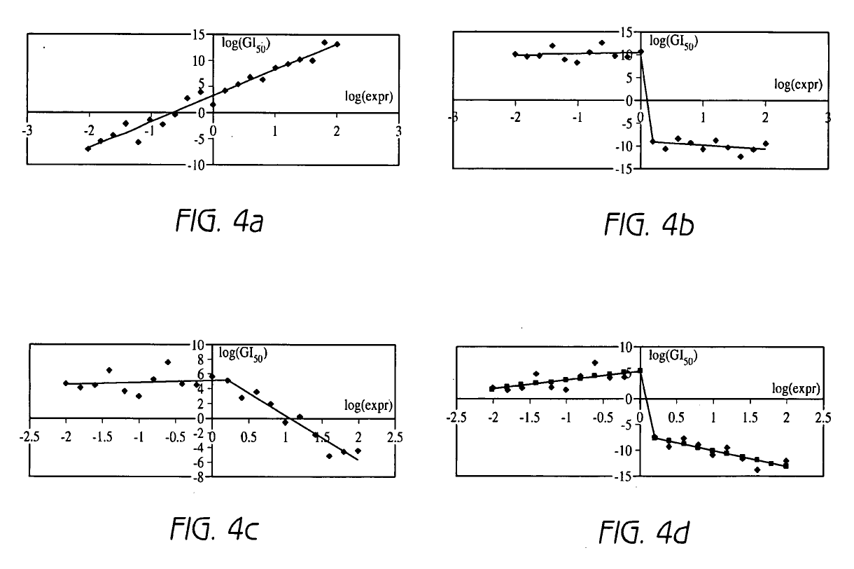

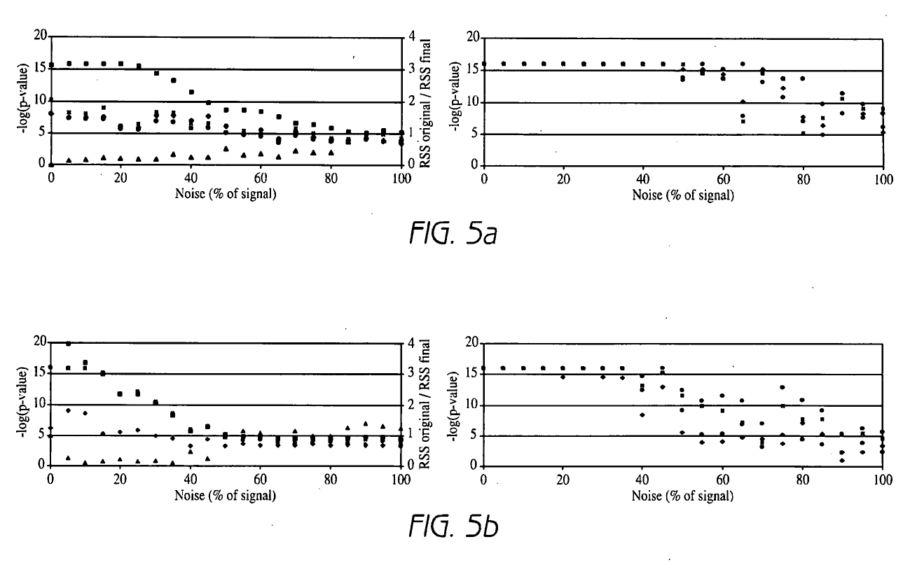

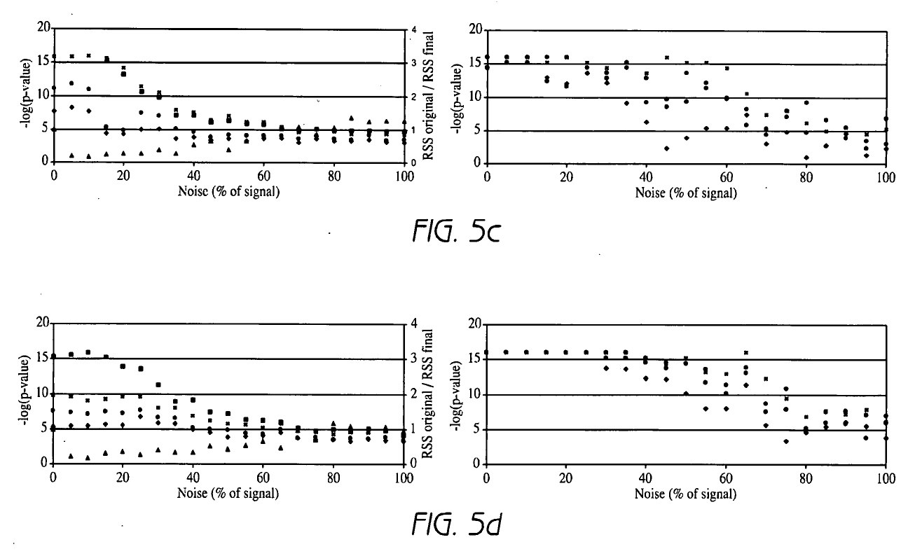

Expression data was obtained for a set of 1000 genes and 30 cell-lines by sampling from a normal distribution with μ=0, and σ=2. These parameters were held fixed. A gene g in the top half of the gene list by variance was randomly selected. The expression level of this gene, {E_{g}}, was then used to generate a model for log(GI₅₀). Four different types of models were explored: (a) Two class model: Here the underlying model had a two class structure, viz. low expressing half of the cell-lines were assigned log(GI₅₀)=5, and the rest were assigned log(GI₅₀)=−5. (b) Linear model: Two random numbers between −1 and +1 were selected representing the slope and intercept of the line, which were then used to compute log(GI₅₀). (c) Single linear spline: Here the model has a two class structure. log(GI₅₀) is constant in one class, and has a linear dependence in the other. The entire function is continuous, representing a single linear spline. The knot of the spline is at the boundary between the two classes. The knot is randomly selected to be in the mid two-thirds of the cell-lines, sorted by expression profile {E_{g}}. The constant and slope are two random numbers between −1 and +1. (d) Linear spline with 2 knots: Here the model again has a two class structure, but log(GI₅₀) has linear dependence in both classes, with a discontinuity at the boundary. Thus, two successive cell-lines, sorted by expression profile {E_{g}}, were used as knots. These were again selected to be in the middle two-thirds of the sorted cell-lines, as before. The highest point of the discontinuity at the knots had log(GI₅₀)=5, while the lowest point had log(GI₅₀)=−5. To avoid complexity and to facilitate controlled studies, the lines were required to have positive slopes <1. (FIG. 4). Noise was added to this model via random numbers obtained from a normal distribution with μ=0, and σ=σ_{G}. The midpoint of the difference between the maximum and minimum values of the pure model above was computed, and σ_{G} was set to a fraction of this difference, the fraction continuously varied (noise (% of signal) in FIG. 5).

Four model types were used to model this data: supervised classification (t-test) and regression methods, viz. linear regression, single linear spline fit and adaptive linear splines. The first three are parametric tests, while adaptive splines constitute a non-parametric test. To perform the t-test, the average log(GI₅₀) was used as a threshold for demarcating the sensitive and resistant classes. Because of the noise, average log(GI₅₀) can be different from the midpoint, which is the actual threshold in the pure model. Expression data from these two groups were used to compute the t statistic. To monitor overfitting effects in the adaptive spline fits, the ratio RSS_{original}/RSS_{final} was recorded, which is greater than 1 when the fitted model is closer to the final input log(GI₅₀) (i.e. with noise) than the original model (i.e. without noise).

For each of these tests, the gene that leads to the highest statistical significance was identified, and the similarity of its expression profile with that of the gene that was originally used to build the GI50 model was assessed. The average of these p-values across nᵢₜₑᵣ=20 iterations are summarized in FIG. 5. Adaptive linear splines model as much variation as the parametric tests in the respective cases, except t-test, which does not model the magnitude of response. Even for the two class scenario, some of the other tests, especially the adaptive spline fit, outperforms the t-test. Though not wishing to be held to any particular theory, this is likely primarily because it does not model the magnitude of response, and uses average log(GI₅₀) as the discriminatory threshold, which can be different from the actual threshold. As shown in FIG. 5, overfitting does not exist when noise is low, and is nominal at high noise, especially in the two-class case. Finally, at high noise, although one does not exactly retrieve the original marker, the similarity of the expression profile with the original marker is typically quite good. Thus, the spline-based method can model various types of response patterns, e.g. bimodal, continuous and other types of patterns, within the same framework, while minimizing the overfitting effects.

### Example 2

### 5-FU Induced Apoptosis in Colon Cancer Cells

Univariate models. To benchmark process 100, it was first applied to the previously published dataset of 5-Fluorouracil (5-FU) induced apoptosis in 30 colon cancer cell-lines (14). Here, only mRNA expression profiles were available as baseline data. Therefore, step 145 of process 100 was not performed. Previous analysis of this dataset involved use of linear regression for univariate correlation, and principal components regression for multivariate modeling.

Using adaptive splines at the univariate level, a total of 48 significant genes that are predictive of apoptotic response (p≦1e-03, FDR=3.7%) (Table 1) were identified. Drug response data was modeled as sum of linear splines, where the predictor variables are DNA amplification, mRNA expression or protein expression levels.

TABLE 1
Significant markers of response to 5-FU induced apoptosis. Comparison of various univariate
tests is shown.

| | | | | | | Present in |
|---|---|---|---|---|---|---|
| | | Adaptive linear | | | Linear | Mariadason |
| | | spline | | t-test | fit | et al (Cancer |
| Id | Description | p-value | q-value | p-value | p-value | Res, 2003)? |
| AA464192 | hypothetical protein | 2.3E−06 | 2.5E−03 | 3.8E−02 | 6.4E−02 | N |
| T95200 | KIAA1250 protein | 2.7E−06 | 2.5E−03 | 6.6E−03 | 4.7E−04 | Y |
| AA464237 | protein phosphatase 4, | 4.4E−06 | 2.7E−03 | 4.2E−03 | 1.8E−04 | Y |
| | regulatory subunit 1 | | | | | |
| AA676604 | MORF-related gene X | 1.2E−05 | 4.9E−03 | 7.7E−03 | 2.8E−05 | Y |
| N36174 | 5-hydroxytryptamine | 1.3E−05 | 4.9E−03 | 2.4E−02 | 1.1E−03 | Y |
| | (serotonin) receptor 2B | | | | | |
| W95041 | heparan sulfate | 1.6E−05 | 4.9E−03 | 9.1E−03 | 1.6E−03 | Y |
| | (glucosamine) 3-O- | | | | | |
| | sulfotransferase 3B1 | | | | | |
| N24910 | cystinosis, nephropathic | 3.6E−05 | 8.5E−03 | 8.8E−01 | 4.4E−01 | N |
| AA428939 | KIAA0095 gene product | 4.0E−05 | 8.5E−03 | 2.9E−01 | 1.8E−01 | N |
| W15386 | ESTs | 4.1E−05 | 8.5E−03 | 2.5E−01 | 3.5E−01 | N |
| AA431749 | ESTs | 7.6E−05 | 1.3E−02 | 5.9E−02 | 2.2E−02 | Y |
| AA401736 | ubiquitously-expressed | 7.9E−05 | 1.3E−02 | 3.3E−02 | 9.6E−02 | N |
| | transcript | | | | | |
| AA669758 | nucleophosmin (nucleolar | 8.3E−05 | 1.3E−02 | 5.8E−02 | 1.2E−03 | Y |
| | phosphoprotein B23, | | | | | |
| | numatrin) | | | | | |
| AA437140 | ESTs, Weakly similar to | 9.4E−05 | 1.3E−02 | 8.9E−02 | 1.6E−02 | Y |
| | B35049 ankyrin 1, | | | | | |
| | erythrocyte splice form 3 - | | | | | |
| | human [H. sapiens] | | | | | |
| AA448285 | cDNA FLJ12946 fis, clone | 1.7E−04 | 2.1E−02 | 7.1E−02 | 1.5E−02 | Y |
| | NT2RP2005254 | | | | | |
| R95893 | EST | 1.7E−04 | 2.1E−02 | 6.7E−02 | 1.9E−02 | Y |
| AA156959 | ceroid-lipofuscinosis, | 2.1E−04 | 2.4E−02 | 6.6E−01 | 9.0E−01 | N |
| | neuronal 5 | | | | | |
| AA045825 | ESTs | 2.4E−04 | 2.5E−02 | 5.5E−02 | 6.2E−03 | Y |
| R17044 | ESTs | 2.4E−04 | 2.5E−02 | 1.1E−01 | 8.9E−01 | N |
| AA022679 | ESTs | 2.9E−04 | 2.8E−02 | 3.3E−03 | 2.9E−04 | Y |
| AA009623 | hypothetical protein | 3.3E−04 | 2.8E−02 | 7.8E−01 | 1.9E−01 | N |
| | FLJ10968 | | | | | |
| AA456595 | ESTs | 3.3E−04 | 2.8E−02 | 1.4E−01 | 2.2E−03 | Y |
| N52651 | cDNA: FLJ22474 fis, clone | 3.4E−04 | 2.8E−02 | 1.8E−02 | 2.6E−04 | Y |
| | HRC10568 | | | | | |
| AA426374 | tubulin, alpha 2 | 4.1E−04 | 3.3E−02 | 3.4E−02 | 4.1E−04 | Y |
| R27319 | ESTs | 4.7E−04 | 3.3E−02 | 5.3E−02 | 1.8E−02 | Y |
| AA496002 | ESTs, Moderately similar to | 4.9E−04 | 3.3E−02 | 3.5E−02 | 1.2E−02 | Y |
| | KIAA1170 protein | | | | | |
| | [H. sapiens] | | | | | |
| AA148536 | nucleoporin 98 kD | 5.0E−04 | 3.3E−02 | 5.1E−02 | 8.9E−03 | Y |
| H42679 | major histocompatibility | 5.1E−04 | 3.3E−02 | 9.0E−01 | 7.3E−01 | N |
| | complex, class II, DM alpha | | | | | |
| AA630346 | KIAA0212 gene product | 5.2E−04 | 3.3E−02 | 2.9E−02 | 2.9E−03 | Y |
| AA130042 | cDNA FLJ12894 fis, clone | 5.3E−04 | 3.3E−02 | 1.1E−01 | 3.0E−02 | Y |
| | NT2RP2004170, | | | | | |
| | moderately similar to | | | | | |
| | mRNA for transducin (beta) | | | | | |
| | like 1 protein | | | | | |
| H99766 | zinc finger protein 24 (KOX | 5.4E−04 | 3.3E−02 | 3.1E−01 | 9.6E−02 | N |
| | 17) | | | | | |
| AA054421 | ring finger protein | 6.0E−04 | 3.4E−02 | 2.0E−01 | 2.9E−02 | Y |
| AA444009 | glucosidase, alpha; acid | 6.2E−04 | 3.4E−02 | 9.4E−01 | 7.9E−01 | N |
| | (Pompe disease, glycogen | | | | | |
| | storage disease type II) | | | | | |
| AA446103 | lectin, mannose-binding, 1 | 6.3E−04 | 3.4E−02 | 1.8E−01 | 8.9E−03 | Y |
| H68885 | tumor suppressing | 6.6E−04 | 3.4E−02 | 1.7E−03 | 6.6E−04 | Y |
| | subtransferable candidate 3 | | | | | |
| AA485214 | nucleobindin 2 | 6.8E−04 | 3.4E−02 | 4.2E−03 | 6.8E−04 | Y |
| AA136666 | cDNA: FLJ22750 fis, clone | 6.9E−04 | 3.4E−02 | 2.0E−02 | 6.3E−03 | Y |
| | KAIA0478 | | | | | |
| H22944 | nicotinamide nucleotide | 6.9E−04 | 3.4E−02 | 5.1E−03 | 5.9E−03 | Y |
| | transhydrogenase | | | | | |
| AA026682 | topoisomerase (DNA) II | 7.1E−04 | 3.4E−02 | 3.6E−02 | 1.6E−03 | Y |
| | alpha (170 kD) | | | | | |
| AA431429 | ESTs, Weakly similar to A- | 7.2E−04 | 3.4E−02 | 3.7E−02 | 1.0E−02 | Y |
| | kinase anchor protein | | | | | |
| | DAKAP550 | | | | | |
| | [D. melanogaster] | | | | | |
| W01084 | hypothetical protein | 7.5E−04 | 3.4E−02 | 2.8E−02 | 5.8E−04 | Y |
| | FLJ10645 | | | | | |
| N52018 | ESTs | 7.9E−04 | 3.6E−02 | 5.3E−01 | 1.5E−01 | N |
| AA872341 | ribosomal protein S15a | 8.3E−04 | 3.6E−02 | 5.5E−01 | 2.2E−01 | N |
| AA427899 | tubulin, beta polypeptide | 8.4E−04 | 3.6E−02 | 7.8E−02 | 3.3E−04 | Y |
| H97765 | clone CDABP0113 mRNA | 8.8E−04 | 3.6E−02 | 1.2E−01 | 3.4E−01 | N |
| | sequence | | | | | |
| AA404694 | PTK2 protein tyrosine | 9.2E−04 | 3.6E−02 | 7.8E−02 | 4.8E−01 | N |
| | kinase 2 | | | | | |
| AA431869 | ubiquitin-conjugating | 9.2E−04 | 3.6E−02 | 3.3E−01 | 4.4E−01 | N |
| | enzyme E2D 2 | | | | | |
| | (homologous to yeast | | | | | |
| | UBC4/5) | | | | | |
| N26536 | ATPase, Cu++ transporting, | 9.2E−04 | 3.6E−02 | 2.2E−02 | 9.2E−04 | Y |
| | beta polypeptide (Wilson | | | | | |
| | disease) | | | | | |
| AA630320 | cDNA DKFZp586C0722 | 9.8E−04 | 3.7E−02 | 4.1E−02 | 5.7E−04 | Y |
| | (from clone | | | | | |
| | DKFZp586C0722) | | | | | |

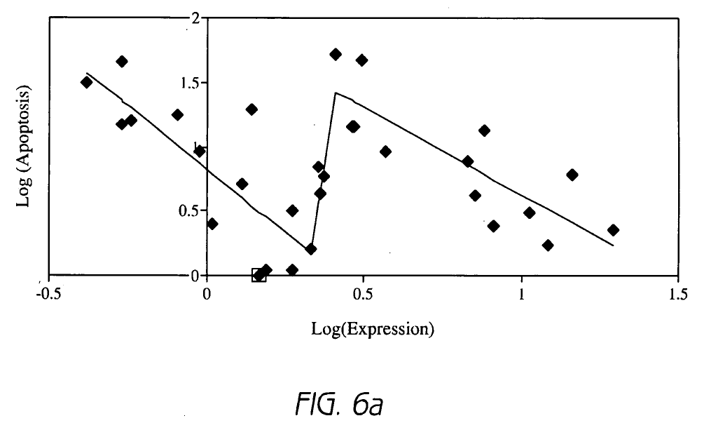

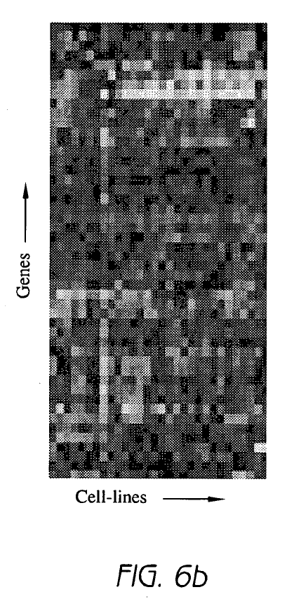

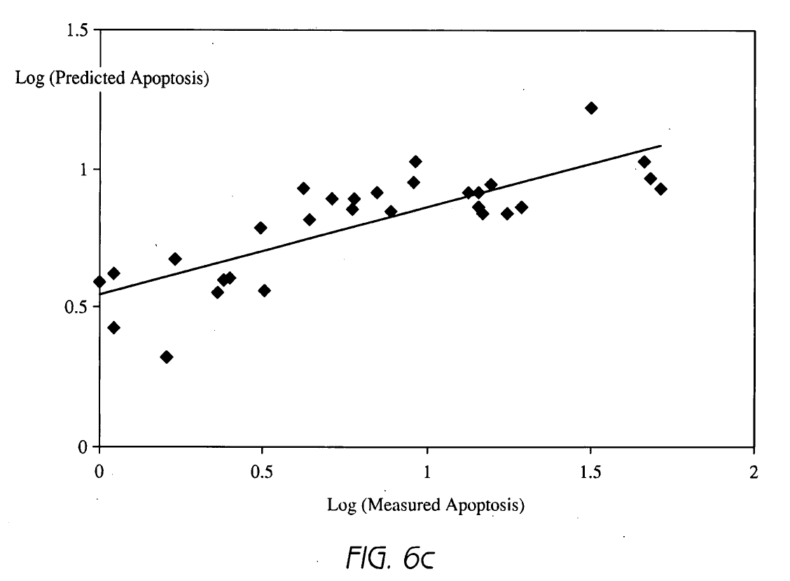

False discovery rate (FDR) was adjusted to ensure ≦2 false discoveries (approximately) throughout this work. The top predictor (PDZD11, FIG. 6a) using splines can capture more variation in the data (p=2e-06) than the linear models used previously (p=3e-05). The average p-value of the top 50 genes using linear splines is 2e-04, while for linear regression, it is 1e-03, again highlighting that adaptive splines can model significantly more variation in the data than the linear methods previously used (Table 1). Of the 48 genes, 32 (=67%) overlap with the previously reported 420 markers (14), the remaining 16 (=33%) are novel. The top predictor, PDZD11, belongs to this set of novel markers. Review of this marker list reveals several molecules (CLN5, CTNS, LYAG) involved in lysosomal processing of macromolecules, indicating possible metabolic determinants of cellular outcome after 5-FU treatment. Some of these genes have been previously associated with cancers: GAA and PTK2 are biomarkers of colonic neoplasms, while RPS15A is known to participate in hepatocellular carcinoma. Functional enrichment analysis of these 48 genes revealed 17 GO terms as significant (p≦0.1), noteworthy among which are macromolecule metabolism, cellular organization and biogenesis, and establishment and maintenance of chromatin architecture (Table 2).

TABLE 2
Significant GO terms enriched among the significant
mRNA markers of 5-FU induced apoptosis.

| GO Term | p-value |
|---|---|
| macromolecule metabolism | 3.7E−04 |
| cell organization and biogenesis | 1.0E−03 |
| metabolism | 1.2E−03 |
| organelle organization and biogenesis | 1.8E−03 |
| protein metabolism | 3.6E−03 |
| intracellular transport | 9.0E−03 |
| establishment of cellular localization | 9.5E−03 |
| cellular localization | 9.8E−03 |
| cellular macromolecule metabolism | 0.02 |
| primary metabolism | 0.02 |
| cytoplasm organization and biogenesis | 0.02 |
| chromatin modification | 0.03 |
| cellular protein metabolism | 0.03 |
| physiological process | 0.05 |
| cellular physiological process | 0.05 |
| cellular metabolism | 0.06 |
| establishment and/or maintenance of | 0.10 |
| chromatin architecture | |

Direct influence of 5-FU on chromatin remodeling has been previously reported. Enrichment analysis with KEGG pathways (See the Internet at “genome.jp/kegg/”) led to gap junction pathway as significant—a pathway that is known to be involved in apoptosis. Unsupervised hierarchical clustering of significant genes clearly shows three distinct groups: first set of genes is high in one group of cell-lines and low in the other set, the second gene set has exactly complementary pattern, while the third set is uniform variation across all cell-lines indicating linear dependencies (FIG. 6b). These distinct class-like patterns could be automatically identified using adaptive linear splines, i.e. without any prior training.

Multivariate models. To obtain a multivariate model, as a start, the most strongly correlated N_{G} univariate predictors were combined using a weighted voting scheme, as described herein. Here, the response of a sample is computed from the weighted average of the predicted magnitude of response from each univariate feature, where the weights of features are proportional to the strength of their univariate correlation. This differs from other methods, where weighted vote of class-type of response was used instead.

The predictive accuracy of the multivariate model is shown using via LOOCV. Here, one cell-line was left out, the model was trained on the remaining 29 cell-lines, and the trained model was used to predict apoptosis on the left-out cell-line. This process was repeated for each of 30 cell-lines. The predictive power of the 48 significant genes at the multivariate level was examined using LOOCV analysis. To seek the upper bound on the accuracy of weighted voting, a different number of predictors (N_{G}) was used at each iteration, the number being that that led to the best performance for that specific iteration. The Pearson's correlation (r) between measured and predicted apoptosis was 0.89 (p=4e-11). To get a more realistic estimate of the power of the weighted voting approach, a set of 1500 genes was created, 500 of which were top predictors of apoptotic response (sorted by splines p-value) and the remaining 1000 were randomly chosen. A representative set of genes was used instead of the complete set to speed up the computation. The LOOCV analysis was then repeated via weighted voting, as above, using only top N_{G} genes, where N_{G} was held fixed through all iterations. The best performance was obtained with N_{G}=6, for which r=0.81 (p=7e-08) between measured and predicted apoptosis (FIG. 6c). Both of these are significantly better than the previously used principal components regression (PCR), which is rooted in linear models. For PCR, r was 0.46 (p=8e-03). From the improved computational performance, it is anticipated that the set of 48 genes constitutes a more robust set of biomarkers of 5-FU induced apoptotic response compared to previous reports.

### Example 3

### Sensitivity to Lapatinib in Breast Cancer Cells

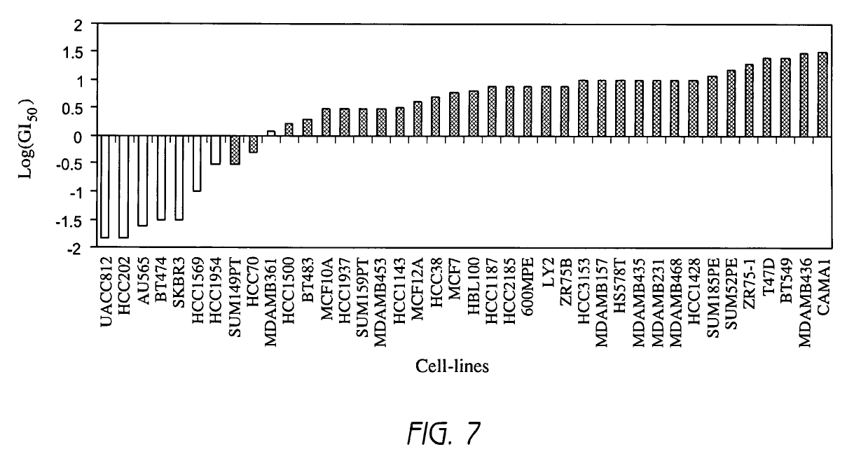

To evaluate the accuracy of a spline-based method as described herein when more than one type of baseline molecular profiles are available, the method was used to model sensitivity of breast cancer cells to Lapatinib, which is a dual inhibitor of epidermal growth factor (EGFR) and HER-2 (ERBB2) tyrosine kinases. DNA copy number changes and protein expression profiles were available, along with the mRNA expression profiles—for a highly characterized model system of breast cancer cell lines. Genome-wide mRNA levels were monitored using Affymetrix U133A arrays, DNA amplification using the array CGH technology, and protein levels using western blot assays. The dose response curves for a total of 40 breast cancer cell lines were determined using the CellTiter Glo assay, which measures cell viability. The response curves were used to estimate the GI₅₀ value for each cell line, which were then used to perform the correlative analyses to predict sensitivity (≡−log(GI₅₀)). The GI₅₀ response data displayed a wide dynamic range (spanning >3 logs) and, as expected, strongly correlated with protein levels of ERBB2, the conventional marker of response to Lapatinib (FIG. 7). To comprehensively determine the predictive markers of sensitivity to Lapatinib, from cell-line panel, a training set of 30 cell-lines was randomly selected. The training set was then used to learn the molecular markers and the computational model for sensitivity prediction. The remaining 10 cell-lines were used to test the accuracy of the model.

Univariate models. Upon application of a linear spline method to this data, it outperformed the previous methods, as in Example 2. For example, for correlation with mRNA profiles, the lowest p-value achieved using the adaptive linear spline test (p=6e-10) is much lower than that obtained by a supervised classification approach, t-test (p=5e-5), or linear regression (p=8e-8), both of which have been used frequently before. The average p-value of the top 50 genes ranked by each of these tests are respectively, 5e-6, 1e-3 and 2e-4, reconfirming that the adaptive linear splines can explain correlations more effectively than the other approaches.

Based on univariate analysis, a total of 155 significant mRNA markers were identified (p≦5e-04, FDR=1.5%), 45 DNA markers from copy number variations (p≦5e-03, FDR=5%) and 9 protein markers (p≦0.01, FDR=1%) (Table 3).

TABLES 3a-c
Response to Lapatinib from each dataset: (a) mRNA expression profiles, (b) DNA
copy number profiles and (c) protein expression profiles. Whether the marker is predictive of
sensitivity or resistance was inferred from the overall directionality of variation.
Table 3(a) mRNA expression

| | | | Predicts | | |
|---|---|---|---|---|---|
| | | | Sensitvity | | |
| | | | (S) or | | |
| Gene | p- | q- | Resistance | Chromosomal | |
| symbol | value | value | (R) | location | Description |
| ERBB2 | 5.8E−10 | 2.4E−06 | S | chr17q11.2-q12\| | v-erb-b2 erythroblastic leukemia |
| | | | | 17q21.1 | viral oncogene homolog 2, |
| | | | | | neuro/glioblastoma derived |
| | | | | | oncogene homolog (avian) |
| GRB7 | 6.1E−10 | 2.4E−06 | S | chr17q12 | growth factor receptor-bound |
| | | | | | protein 7 |
| AYTL2 | 6.2E−07 | 1.0E−03 | S | chr5p15.33 | acyltransferase like 2 |
| STARD3 | 8.6E−07 | 1.0E−03 | S | chr17q11-q12 | START domain containing 3 |
| NCOA6 | 1.1E−06 | 1.1E−03 | S | chr20q11 | nuclear receptor coactivator 6 |
| RPL19 | 1.3E−06 | 1.2E−03 | S | chr17q11.2-q12 | ribosomal protein L19 /// ribosomal |
| | | | | | protein L19 |
| TLE3 | 1.6E−06 | 1.3E−03 | S | chr15q22 | transducin-like enhancer of split 3 |
| | | | | | (E(sp1) homolog, Drosophila) |
| PERLD1 | 2.1E−06 | 1.4E−03 | S | chr17q12 | per1-like domain containing 1 |
| SLC35A2 | 2.4E−06 | 1.4E−03 | S | chrXp11.23-p11.22 | solute carrier family 35 (UDP- |
| | | | | | galactose transporter), member |
| | | | | | A2 |
| PSMD3 | 2.6E−06 | 1.4E−03 | S | chr17q12-q21.1 | proteasome (prosome, macropain) |
| | | | | | 26S subunit, non-ATPase, 3 |
| TRA2A | 3.0E−06 | 1.4E−03 | S | chr7p15.3 | transformer-2 alpha |
| KIAA0232 | 3.1E−06 | 1.4E−03 | S | chr4p16.1 | KIAA0232 gene product |
| PSMC3 | 3.8E−06 | 1.5E−03 | R | chr11p12-p13 | proteasome (prosome, macropain) |
| | | | | | 26S subunit, ATPase, 3 |
| FBXO2 | 3.9E−06 | 1.5E−03 | S | chr1p36.22 | F-box protein 2 |
| PCGF2 | 4.1E−06 | 1.5E−03 | S | chr17q12 | polycomb group ring finger 2 |
| TMEM132A | 4.3E−06 | 1.5E−03 | S | chr11q12.2 | transmembrane protein 132A |
| C16orf58 | 5.1E−06 | 1.6E−03 | S | chr16p11.2 | chromosome 16 open reading |
| | | | | | frame 58 |
| THRAP4 | 5.4E−06 | 1.7E−03 | S | chr17q21.1 | thyroid hormone receptor |
| | | | | | associated protein 4 |
| VIM | 7.0E−06 | 1.9E−03 | R | chr10p13 | vimentin |
| LRP16 | 7.2E−06 | 1.9E−03 | S | chr11q11 | LRP16 protein |
| MAP3K14 | 7.3E−06 | 1.9E−03 | R | chr17q21 | mitogen-activated protein kinase |
| | | | | | kinase kinase 14 |
| GSDML | 7.3E−06 | 1.9E−03 | S | chr17q12 | gasdermin-like |
| 43511_s_at | 7.5E−06 | 1.9E−03 | S | — | MRNA; cDNA DKFZp762M127 |
| | | | | | (from clone DKFZp762M127) |
| C20orf43 | 8.3E−06 | 1.9E−03 | S | chr20q13.31 | chromosome 20 open reading |
| | | | | | frame 43 |
| PRSS22 | 8.4E−06 | 1.9E−03 | S | chr16p13.3 | protease, serine, 22 |
| C14orf161 | 8.6E−06 | 1.9E−03 | S | chr14q32.12 | chromosome 14 open reading |
| | | | | | frame 161 |
| LOC645619 | 8.9E−06 | 1.9E−03 | S | chr12p11.21 | similar to Adenylate kinase |
| | | | | | isoenzyme 4, mitochondrial (ATP- |
| | | | | | AMP transphosphorylase) |
| C16orf34 | 1.1E−05 | 2.2E−03 | S | chr16p13.3 | chromosome 16 open reading |
| | | | | | frame 34 |
| VDP | 1.1E−05 | 2.2E−03 | S | chr4q21.1 | Vesicle docking protein p115 |
| TFAP2C | 1.2E−05 | 2.2E−03 | S | chr20q13.2 | transcription factor AP-2 gamma |
| | | | | | (activating enhancer binding |
| | | | | | protein 2 gamma) |
| 213785_at | 1.3E−05 | 2.2E−03 | S | — | MRNA; cDNA DKFZp686P1617 |
| | | | | | (from clone DKFZp686P1617) |
| CBX5 | 1.5E−05 | 2.4E−03 | R | chr12q13.13 | chromobox homolog 5 (HP1 alpha |
| | | | | | homolog, Drosophila) |
| TAX1BP1 | 1.5E−05 | 2.4E−03 | S | chr7p15 | Tax1 (human T-cell leukemia |
| | | | | | virus type I) binding protein 1 |
| CALCOCO2 | 1.9E−05 | 2.8E−03 | S | chr17q21.32 | calcium binding and coiled-coil |
| | | | | | domain 2 |
| NIP7 | 1.9E−05 | 2.8E−03 | R | chr16q22.1 | nuclear import 7 homolog (S. cerevisiae) |
| PHLPP | 2.0E−05 | 2.8E−03 | S | chr18q21.33 | PH domain and leucine rich repeat |
| | | | | | protein phosphatase |
| VEGF | 2.0E−05 | 2.8E−03 | S | chr6p12 | vascular endothelial growth factor |
| UBE1 | 2.1E−05 | 2.8E−03 | S | chrXp11.23 | ubiquitin-activating enzyme E1 |
| | | | | | (A1S9T and BN75 temperature |
| | | | | | sensitivity complementing) |
| GOSR1 | 2.3E−05 | 3.1E−03 | S | chr17q11 | golgi SNAP receptor complex |
| | | | | | member 1 |
| YARS | 2.4E−05 | 3.1E−03 | R | chr1p35.1 | tyrosyl-tRNA synthetase |
| LOC401034 | 2.4E−05 | 3.1E−03 | S | chr2q37.1 | hypothetical LOC401034 |
| SUOX | 2.4E−05 | 3.1E−03 | S | chr12q13.2 | sulfite oxidase |
| ITGBL1 | 2.5E−05 | 3.1E−03 | R | chr13q33 | integrin, beta-like 1 (with EGF-like |
| | | | | | repeat domains) |
| 49111_at | 2.6E−05 | 3.1E−03 | S | — | MRNA; cDNA DKFZp762M127 |
| | | | | | (from clone DKFZp762M127) |
| KIAA0100 | 2.6E−05 | 3.1E−03 | S | chr17q11.2 | KIAA0100 |
| CSTF1 | 2.8E−05 | 3.3E−03 | S | chr20q13.31 | cleavage stimulation factor, 3′ pre- |
| | | | | | RNA, subunit 1, 50 kDa |
| ERAL1 | 2.9E−05 | 3.4E−03 | S | chr17q11.2 | Era G-protein-like 1 (E. coli) |
| UNG | 3.1E−05 | 3.5E−03 | S | chr12q23-q24.1 | uracil-DNA glycosylase |
| ARHGAP8 | 3.1E−05 | 3.5E−03 | S | chr22q13.31 /// | Rho GTPase activating protein 8 |
| /// | | | | chr22q13 | /// PRR5-ARHGAP8 fusion |
| LOC553158 | | | | | |
| CRKRS | 3.5E−05 | 3.8E−03 | S | chr17q12 | Cdc2-related kinase, |
| | | | | | arginine/serine-rich |
| PIK3C2A | 3.6E−05 | 3.8E−03 | S | chr11p15.5-p14 | phosphoinositide-3-kinase, class |
| | | | | | 2, alpha polypeptide |
| GALNT2 | 3.6E−05 | 3.8E−03 | R | chr1q41-q42 | UDP-N-acetyl-alpha-D- |
| | | | | | galactosamine:polypeptide N- |
| | | | | | acetylgalactosaminyltransferase 2 |
| | | | | | (GalNAc-T2) |
| KRT19 | 3.7E−05 | 3.8E−03 | S | chr17q21.2 | keratin 19 |
| FLJ11184 | 4.3E−05 | 4.2E−03 | R | chr4q32.3 | hypothetical protein FLJ11184 |
| MAL | 4.6E−05 | 4.4E−03 | S | chr2cen-q13 | mal, T-cell differentiation protein |
| HCA112 | 4.9E−05 | 4.7E−03 | S | chr7q36.1 | hepatocellular carcinoma- |
| | | | | | associated antigen 112 |
| SPRY2 | 5.1E−05 | 4.7E−03 | R | chr13q31.1 | sprouty homolog 2 (Drosophila) |
| CASD1 | 5.2E−05 | 4.8E−03 | S | chr7q21.3 | CAS1 domain containing 1 |
| CST3 | 5.9E−05 | 5.3E−03 | S | chr20p11.21 | cystatin C (amyloid angiopathy |
| | | | | | and cerebral hemorrhage) |
| ANKRD17 | 6.4E−05 | 5.6E−03 | S | chr4q13.3 | ankyrin repeat domain 17 |
| WDR68 | 6.4E−05 | 5.6E−03 | R | chr17q23.3 | WD repeat domain 68 |
| PPARBP | 6.7E−05 | 5.7E−03 | S | chr17q12-q21.1 | PPAR binding protein |
| FGG | 6.8E−05 | 5.7E−03 | S | chr4q28 | fibrinogen gamma chain |
| MSTO1 | 6.9E−05 | 5.7E−03 | S | chr1q22 | misato homolog 1 (Drosophila) |
| CTNNB1 | 7.4E−05 | 6.0E−03 | R | chr3p21 | catenin (cadherin-associated |
| | | | | | protein), beta 1, 88 kDa |
| ARHGEF5 | 8.2E−05 | 6.5E−03 | S | chr7q33-q35 | Rho guanine nucleotide exchange |
| | | | | | factor (GEF) 5 |
| WRB | 9.1E−05 | 7.0E−03 | R | chr21q22.3 | tryptophan rich basic protein |
| FAM13A1 | 9.1E−05 | 7.0E−03 | S | chr4q22.1 | family with sequence similarity 13, |
| | | | | | member A1 |
| SEPT8 | 9.2E−05 | 7.0E−03 | S | chr5q31 | septin 8 |
| SLC16A1 | 9.7E−05 | 7.3E−03 | R | chr1p12 | solute carrier family 16, member 1 |
| | | | | | (monocarboxylic acid transporter |
| | | | | | 1) |
| SUPT6H | 9.9E−05 | 7.3E−03 | S | chr17q11.2 | suppressor of Ty 6 homolog (S. cerevisiae) |
| CANT1 | 1.1E−04 | 7.6E−03 | S | chr17q25.3 | calcium activated nucleotidase 1 |
| KRT15 | 1.1E−04 | 7.6E−03 | S | chr17q21.2 | keratin 15 |
| RAB26 | 1.1E−04 | 7.6E−03 | S | chr16p13.3 | RAB26, member RAS oncogene |
| | | | | | family |
| RBBP4 | 1.1E−04 | 7.8E−03 | S | chr1p35.1 | retinoblastoma binding protein 4 |
| APOBEC3C | 1.2E−04 | 8.0E−03 | R | chr22q13.1-q13.2 | apolipoprotein B mRNA editing |
| | | | | | enzyme, catalytic polypeptide-like |
| | | | | | 3C |
| ENTPD6 | 1.2E−04 | 8.0E−03 | S | chr20p11.2-p11.22 | ectonucleoside triphosphate |
| | | | | | diphosphohydrolase 6 (putative |
| | | | | | function) |
| EMP3 | 1.3E−04 | 8.6E−03 | R | chr19q13.3 | epithelial membrane protein 3 |
| PLXNA3 | 1.4E−04 | 8.6E−03 | S | chrXq28 | plexin A3 |
| MGAT4A | 1.4E−04 | 8.6E−03 | S | chr2q12 | mannosyl (alpha-1,3-)- |
| | | | | | glycoprotein beta-1,4-N- |
| | | | | | acetylglucosaminyltransferase, |
| | | | | | isozyme A |
| PSMD4 | 1.4E−04 | 8.6E−03 | R | chr1q21.2 | proteasome (prosome, macropain) |
| | | | | | 26S subunit, non-ATPase, 4 |
| KIAA1718 | 1.4E−04 | 8.6E−03 | S | chr7q34 | KIAA1718 protein |
| OSR2 | 1.4E−04 | 8.6E−03 | S | chr8q22.2 | odd-skipped related 2 (Drosophila) |
| FECH | 1.6E−04 | 9.6E−03 | S | chr18q21.3 | ferrochelatase (protoporphyria) |
| CPE | 1.6E−04 | 9.6E−03 | S | chr4q32.3 | carboxypeptidase E |
| SF3B1 | 1.7E−04 | 9.6E−03 | R | chr2q33.1 | splicing factor 3b, subunit 1, |
| | | | | | 155 kDa |
| FLJ30092 | 1.7E−04 | 9.6E−03 | S | chr12q24.13 | AF-1 specific protein phosphatase |
| | | | | | /// AF-1 specific protein |
| | | | | | phosphatase |
| SEMA4C | 1.7E−04 | 9.9E−03 | S | chr2q11.2 | sema domain, immunoglobulin |
| | | | | | domain (Ig), transmembrane |
| | | | | | domain (TM) and short |
| | | | | | cytoplasmic domain, (semaphorin) |
| | | | | | 4C |
| 213048_s_at | 1.7E−04 | 9.9E−03 | R | — | MRNA from HIV associated non- |
| | | | | | Hodgkin's lymphoma (clone hl1-98) |
| EFNB2 | 1.8E−04 | 1.0E−02 | R | chr13q33 | ephrin-B2 |
| CST4 | 1.8E−04 | 1.0E−02 | S | chr20p11.21 | cystatin S |
| TFPI2 | 1.9E−04 | 1.0E−02 | R | chr7q22 | tissue factor pathway inhibitor 2 |
| IFIT1 | 1.9E−04 | 1.0E−02 | R | chr10q25-q26 | interferon-induced protein with |
| | | | | | tetratricopeptide repeats 1 /// |
| | | | | | interferon-induced protein with |
| | | | | | tetratricopeptide repeats 1 |
| FAM89B | 1.9E−04 | 1.0E−02 | S | chr11q23 | family with sequence similarity 89, |
| | | | | | member B |
| PPFIBP1 | 2.0E−04 | 1.1E−02 | R | chr12p11.23-p11.22 | PTPRF interacting protein, binding |
| | | | | | protein 1 (liprin beta 1) |
| TIAF1 /// | 2.0E−04 | 1.1E−02 | S | chr17q11.2 | TGFB1-induced anti-apoptotic |
| MYO18A | | | | | factor 1 /// myosin XVIIIA |
| WIRE | 2.0E−04 | 1.1E−02 | S | chr17q21.2 | WIRE protein |
| LXN | 2.0E−04 | 1.1E−02 | S | chr3q25.32 | latexin |
| DKFZp586I1420 | 2.1E−04 | 1.1E−02 | S | chr7p15.1 | hypothetical protein |
| | | | | | DKFZp586I1420 |
| COL9A2 | 2.1E−04 | 1.1E−02 | S | chr1p33-p32 | collagen, type IX, alpha 2 |
| CSTB | 2.2E−04 | 1.1E−02 | R | chr21q22.3 | cystatin B (stefin B) |
| CGA | 2.2E−04 | 1.1E−02 | S | chr6q12-q21 | glycoprotein hormones, alpha |
| | | | | | polypeptide |
| RP13- | 2.3E−04 | 1.1E−02 | S | chrXp22.32; | DNA segment on chromosome X |
| 297E16.1 | | | | Ypter-p11.2 | and Y (unique) 155 expressed |
| | | | | | sequence, isoform 1 |
| EFNA1 | 2.3E−04 | 1.1E−02 | S | chr1q21-q22 | ephrin-A1 |
| WSB1 | 2.4E−04 | 1.1E−02 | S | chr17q11.1 | WD repeat and SOCS box- |
| | | | | | containing 1 |
| C19orf58 | 2.4E−04 | 1.1E−02 | S | chr19p13.11 | chromosome 19 open reading |
| | | | | | frame 58 |
| LOC651633 | 2.4E−04 | 1.1E−02 | S | — | similar to Rho-associated protein |
| | | | | | kinase 1 (Rho-associated, coiled- |
| | | | | | coil containing protein kinase 1) |
| | | | | | (p160 ROCK-1) (p160ROCK) |
| CYFIP1 | 2.5E−04 | 1.2E−02 | S | chr15q11 | cytoplasmic FMR1 interacting |
| | | | | | protein 1 |
| NUP43 | 2.5E−04 | 1.2E−02 | S | chr6q25.1 | nucleoporin 43 kDa |
| PAFAH1B1 | 2.6E−04 | 1.2E−02 | R | chr17p13.3 | Platelet-activating factor |
| | | | | | acetylhydrolase, isoform lb, alpha |
| | | | | | subunit 45 kDa |
| MRPL22 | 2.6E−04 | 1.2E−02 | R | chr5q33.1-q33.3 | mitochondrial ribosomal protein |
| | | | | | L22 |
| ARPC2 | 2.7E−04 | 1.2E−02 | R | chr2q36.1 | actin related protein 2/3 complex, |
| | | | | | subunit 2, 34 kDa |
| TRPM2 | 2.8E−04 | 1.2E−02 | S | chr21q22.3 | transient receptor potential cation |
| | | | | | channel, subfamily M, member 2 |
| TSPAN13 | 2.8E−04 | 1.2E−02 | S | chr7p21.1 | Tetraspanin 13 |
| C6orf111 | 2.8E−04 | 1.2E−02 | S | chr6q16.3 | chromosome 6 open reading |
| | | | | | frame 111 |
| DLG7 | 2.8E−04 | 1.2E−02 | R | chr14q22.3 | discs, large homolog 7 |
| | | | | | (Drosophila) |
| PGF | 2.8E−04 | 1.2E−02 | R | chr14q24-q31 | placental growth factor, vascular |
| | | | | | endothelial growth factor-related |
| | | | | | protein |
| RPN2 | 2.9E−04 | 1.2E−02 | S | chr20q12-q13.1 | ribophorin II |
| RAB6IP1 | 2.9E−04 | 1.2E−02 | R | chr11p15.4 | RAB6 interacting protein 1 |
| SPAG5 | 3.0E−04 | 1.2E−02 | S | chr17q11.2 | sperm associated antigen 5 |
| DNAJC8 | 3.1E−04 | 1.3E−02 | R | chr1p35.3 | DnaJ (Hsp40) homolog, subfamily |
| | | | | | C, member 8 |
| P4HB | 3.1E−04 | 1.3E−02 | S | chr17q25 | procollagen-proline, 2- |
| | | | | | oxoglutarate 4-dioxygenase |
| | | | | | (proline 4-hydroxylase), beta |
| | | | | | polypeptide |
| TRAF4 | 3.1E−04 | 1.3E−02 | S | chr17q11-q12 | TNF receptor-associated factor 4 |
| CRI1 | 3.1E−04 | 1.3E−02 | R | chr15q21.1-q21.2 | CREBBP/EP300 inhibitor 1 |
| RARA | 3.1E−04 | 1.3E−02 | S | chr17q21 | retinoic acid receptor, alpha |
| AKR1B1 | 3.1E−04 | 1.3E−02 | R | chr7q35 | aldo-keto reductase family 1, |
| | | | | | member B1 (aldose reductase) |
| GMDS | 3.3E−04 | 1.3E−02 | S | chr6p25 | GDP-mannose 4,6-dehydratase |
| LBP | 3.4E−04 | 1.3E−02 | S | chr20q11.23-q12 | lipopolysaccharide binding protein |
| TNFAIP1 | 3.4E−04 | 1.3E−02 | S | chr17q22-q23 | tumor necrosis factor, alpha- |
| | | | | | induced protein 1 (endothelial) |
| RAB1B | 3.4E−04 | 1.3E−02 | S | chr11q12 | RAB1B, member RAS oncogene |
| | | | | | family /// RAB1B, member RAS |
| | | | | | oncogene family |
| HMGB1 | 3.5E−04 | 1.3E−02 | R | chr13q12 | high-mobility group box 1 |
| HIST1H2AM | 3.5E−04 | 1.3E−02 | R | chr6p22-p21.3 | histone 1, H2am |
| RGL2 | 3.6E−04 | 1.4E−02 | S | chr6p21.3 | ral guanine nucleotide dissociation |
| | | | | | stimulator-like 2 |
| SEC13L1 | 3.9E−04 | 1.4E−02 | R | chr3p25-p24 | SEC13-like 1 (S. cerevisiae) |
| MMD | 4.0E−04 | 1.5E−02 | S | chr17q | monocyte to macrophage |
| | | | | | differentiation-associated |
| ARHGEF10 | 4.1E−04 | 1.5E−02 | R | chr8p23 | Rho guanine nucleotide exchange |
| | | | | | factor (GEF) 10 |
| CGREF1 | 4.1E−04 | 1.5E−02 | S | chr2p23.3 | cell growth regulator with EF-hand |
| | | | | | domain 1 |
| LOC339287 | 4.3E−04 | 1.5E−02 | S | chr17q21.1 | hypothetical protein LOC339287 |
| PIN1 | 4.3E−04 | 1.5E−02 | R | chr19p13 | protein (peptidylprolyl cis/trans |
| | | | | | isomerase) NIMA-interacting 1 |
| CXX1 | 4.4E−04 | 1.5E−02 | R | chrXq26 | CAAX box 1 |
| ZBED1 | 4.5E−04 | 1.5E−02 | S | chrXp22.33; Yp11 | zinc finger, BED-type containing 1 |
| SNX13 | 4.5E−04 | 1.5E−02 | S | chr7p21.1 | sorting nexin 13 |
| RGS2 | 4.5E−04 | 1.5E−02 | R | chr1q31 | regulator of G-protein signalling 2, |
| | | | | | 24 kDa |
| PSMD11 | 4.6E−04 | 1.6E−02 | R | chr17q11.2 | proteasome (prosome, macropain) |
| | | | | | 26S subunit, non-ATPase, 11 |
| GNAS | 4.6E−04 | 1.6E−02 | S | chr20q13.3 | GNAS complex locus |
| STX16 | 4.7E−04 | 1.6E−02 | S | chr20q13.32 | syntaxin 16 |
| NEO1 | 4.7E−04 | 1.6E−02 | S | chr15q22.3-q23 | neogenin homolog 1 (chicken) |
| HMGB3 | 4.7E−04 | 1.6E−02 | S | chrXq28 | high-mobility group box 3 |
| PLXNB2 | 4.8E−04 | 1.6E−02 | S | chr22q13.33 | plexin B2 |
| RPL14 /// | 4.8E−04 | 1.6E−02 | R | chr3p22-p21.2 /// | ribosomal protein L14 /// ribosomal |
| RPL14L /// | | | | chr12q14.2 | protein L14 /// ribosomal protein |
| LOC649821 | | | | | L14-like /// ribosomal protein L14- |
| | | | | | like /// similar to 60S ribosomal |
| | | | | | protein L14 (CAG-ISL 7) /// similar |
| | | | | | to 60S ribosomal protein L14 |
| | | | | | (CAG-ISL 7) |
| ATP6AP1 | 4.9E−04 | 1.6E−02 | S | chrXq28 | ATPase, H+ transporting, |
| | | | | | lysosomal accessory protein 1 |
| CYP2B7P1 | 4.9E−04 | 1.6E−02 | S | chr19q13.2 | cytochrome P450, family 2, |
| | | | | | subfamily B, polypeptide 7 |
| | | | | | pseudogene 1 |
| TPI1 | 4.9E−04 | 1.6E−02 | S | chr12p13 | triosephosphate isomerase 1 |
| KTN1 | 4.9E−04 | 1.6E−02 | S | chr14q22.1 | kinectin 1 (kinesin receptor) |
| EMP1 | 4.9E−04 | 1.6E−02 | R | chr12p12.3 | epithelial membrane protein 1 |

Table 3(b) Copy number

| | | | | Predicts |
|---|---|---|---|---|
| | | | | Sensitivity (S) |
| | | | | or Resistance |
| Clone Id | Chromosome_kb_kbGenome | p-value | q-value | (R) |
| RP11-62N23 | 17_38047.53_2522294.65 | 7.1E−10 | 8.7E−07 | S |
| RMC17P077 | 17_38259.651_2522506.771 | 1.0E−09 | 8.7E−07 | S |
| DMPC-HFF#1- | 17_38256.761_2522503.881 | 5.4E−07 | 2.3E−04 | S |
| 61H8 | | | | |
| CTD-2094C6 | 17_38620.672_2522867.792 | 1.2E−05 | 1.8E−03 | S |
| CTC-329F6 | 7_2140.969_1234910.375 | 1.3E−05 | 1.8E−03 | S |
| CTD-2174G23 | 20_35925.95_2741960.126 | 1.4E−05 | 1.8E−03 | S |
| RP11-212M6 | 20_53690.882_2759725.058 | 2.1E−05 | 2.2E−03 | S |
| RP11-110H20 | 17_47395.499_2531642.619 | 2.3E−05 | 2.2E−03 | S |
| RP11-55E1 | 20_53054.082_2759088.258 | 2.4E−05 | 2.2E−03 | S |
| RP11-23D23 | 7_807.055_1233576.461 | 2.5E−05 | 2.2E−03 | S |
| RMC20B4130 | 20_52853.96_2758888.136 | 2.6E−05 | 2.2E−03 | S |
| RP11-749I16 | 17_38586.245_2522833.365 | 2.8E−05 | 2.4E−03 | S |
| RP11-55D2 | 20_53186.348_2759220.524 | 3.1E−05 | 2.4E−03 | S |
| RMC20P070 | 20_53690.882_2759725.058 | 5.6E−05 | 3.9E−03 | S |
| RMC20P037 | 20_36697.499_2742731.675 | 1.0E−04 | 6.7E−03 | S |
| GS-32I19 | 20_55629.949_2761664.125 | 1.8E−04 | 9.2E−03 | S |
| RMC20P160 | 20_10281.845_2716316.021 | 2.0E−04 | 9.3E−03 | S |
| RMC20B4087 | 20_53455.409_2759489.585 | 3.2E−04 | 1.3E−02 | S |
| RP11-87N6 | 17_38680.669_2522927.789 | 3.4E−04 | 1.4E−02 | S |
| LLNL-255K9 | 20_56590.236_2762624.412 | 3.8E−04 | 1.4E−02 | S |
| RP11-138A15 | 20_36735.699_2742769.875 | 4.3E−04 | 1.5E−02 | S |
| RP11-128B23 | 23_18196.45_2884345.563 | 4.9E−04 | 1.6E−02 | S |
| RP11-126A13 | 23_54383.804_2920532.917 | 5.7E−04 | 1.7E−02 | S |
| GS-265E19 | 17_38917.959_2523165.079 | 6.1E−04 | 1.8E−02 | S |
| GS1-35C5 | 7_69637.921_1302407.327 | 6.3E−04 | 1.8E−02 | S |
| RP11-186B13 | 18_49164.662_2615272.048 | 6.4E−04 | 1.8E−02 | S |
| CTD-2033A1 | 23_21571.995_2887721.108 | 6.7E−04 | 1.8E−02 | S |
| RP11-58O9 | 17_38874.35_2523121.47 | 6.7E−04 | 1.8E−02 | S |
| RP11-146L11 | 20_53245.938_2759280.114 | 8.0E−04 | 2.0E−02 | S |
| LLNLBAC-255K9 | 20_56590.236_2762624.412 | 1.1E−03 | 2.3E−02 | S |
| RMC20P073 | 20_56821.557_2762855.733 | 1.3E−03 | 2.5E−02 | S |
| RMC17P034 | 17_38860.534_2523107.654 | 1.3E−03 | 2.5E−02 | S |
| RP11-14K11 | 7_54870.929_1287640.335 | 1.9E−03 | 3.1E−02 | S |
| RP11-133E8 | 20_52869.034_2758903.21 | 2.0E−03 | 3.2E−02 | S |
| RP11-124H12 | 23_31162.885_2897311.998 | 2.2E−03 | 3.4E−02 | S |
| CTD-2232D15 | 20_42981.137_2749015.313 | 2.2E−03 | 3.4E−02 | S |
| CTD-2005M12 | 8_118644.713_1509959.637 | 2.3E−03 | 3.4E−02 | S |
| RP11-50F16 | 17_58501.845_2542748.965 | 2.6E−03 | 3.5E−02 | S |
| RP11-321B9 | 7_10936.527_1243705.933 | 2.8E−03 | 3.6E−02 | S |
| RP11-19L3 | 18_42180.646_2608288.032 | 3.2E−03 | 3.7E−02 | S |
| CTD-2002E24 | 23_36499.949_2902649.062 | 3.3E−03 | 3.8E−02 | S |
| CTC-215O4 | 19_10849.981_2653072.506 | 4.3E−03 | 4.5E−02 | R |
| GS-236D3 | 20_49884.662_2755918.838 | 4.4E−03 | 4.5E−02 | S |
| RP11-43K24 | 18_45620.759_2611728.145 | 4.4E−03 | 4.5E−02 | S |
| RP11-124D1 | 20_47923.743_2753957.919 | 4.7E−03 | 4.7E−02 | S |

Table 3(c) Western Blots

| | p- | q- | |
|---|---|---|---|
| Protein Id | value | value | Predicts Sensitivity (S) or Resistance (R) |
| ERBB2-P | 9.5E−09 | 1.5E−07 | S |
| ERBB2 | 3.6E−07 | 2.9E−06 | S |
| GRB7 | 1.9E−06 | 1.0E−05 | S |
| EFNA1 | 1.8E−04 | 7.3E−04 | S |
| JAK1 | 6.3E−04 | 2.0E−03 | R |
| ESR1 | 2.4E−03 | 6.3E−03 | S |
| FLNA_UP | 3.5E−03 | 8.1E−03 | R |
| PTK2 | 7.3E−03 | 1.3E−02 | R |
| MDM2 | 7.3E−03 | 1.3E−02 | S |

ERBB2, the canonical marker of response to Lapatinib (REF), is consistently represented among the top predictors across all data sets. The ERBB2 amplicon (Chr 17q21) and phosphor-ERBB2 are also the top predictors in DNA amplification data and western blot data respectively. These analyses show the same ERBB2 specificity as observed in clinical trials and in other in vitro experiments. The positive associations of ERBB2 with sensitivity were expected because it is a principal target of Lapatinib. The same is true of genes encoded in the ERBB2 amplicon (e.g. GRB7), since they are co-amplified and over-expressed with ERBB2 in these tumors. However, this result is an important validation of the association analysis in that it does select ERBB2 and the co-amplified genes as the most important predictors of response.

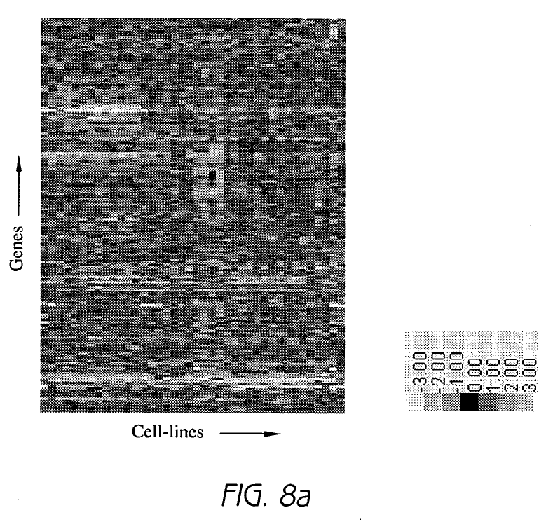

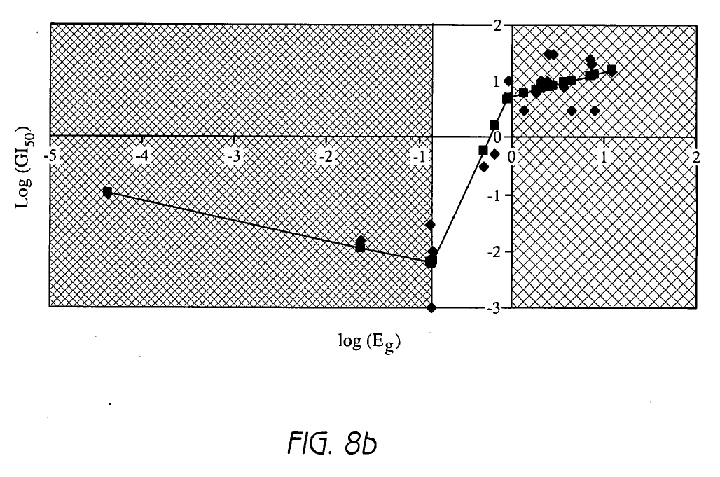

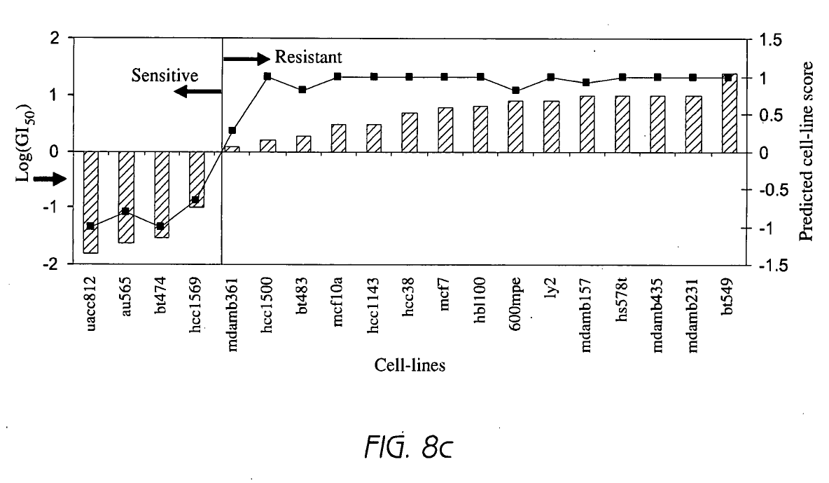

The 155 significant mRNA markers were clustered by their expression levels using unsupervised hierarchical clustering. The genes automatically separated into two distinct groups, characteristic of resistant and sensitive classes (FIG. 8a), reconfirming the notion that linear splines can naturally identify class-like features without any training. Furthermore, functional enrichment analysis of the significant mRNA markers using GO terms was performed (Table 4).

TABLE 4
Significant GO terms enriched among mRNA
markers of response to Lapatinib.

| GO term | p-value |
|---|---|
| cell death | 1.7E−03 |
| death | 1.8E−03 |
| cell organization and biogenesis | 2.8E−03 |
| DNA replication | 4.8E−03 |
| steroid hormone receptor signaling pathway | 4.8E−03 |
| intracellular signaling cascade | 5.3E−03 |
| intracellular receptor-mediated signaling | 5.5E−03 |
| pathway | |
| positive regulation of cellular physiological | 8.3E−03 |
| process | |
| positive regulation of cellular process | 8.6E−03 |
| positive regulation of physiological process | 1.1E−02 |
| DNA metabolism | 1.5E−02 |
| protein localization | 1.7E−02 |
| cell communication | 1.9E−02 |
| transmembrane receptor protein tyrosine kinase | 2.0E−02 |
| signaling pathway | |
| cellular localization | 2.1E−02 |
| cell proliferation | 2.3E−02 |
| positive regulation of biological process | 2.4E−02 |
| androgen receptor signaling pathway | 2.6E−02 |
| organ morphogenesis | 2.6E−02 |
| regulation of cell proliferation | 2.8E−02 |
| apoptosis | 2.9E−02 |
| transcription initiation from RNA polymerase II | 2.9E−02 |
| promoter | |
| programmed cell death | 2.9E−02 |
| DNA repair | 3.4E−02 |
| establishment of protein localization | 3.5E−02 |
| regulation of cellular process | 3.5E−02 |
| locomotion | 3.7E−02 |
| localization of cell | 3.7E−02 |
| cell motility | 3.7E−02 |
| morphogenesis | 4.1E−02 |
| establishment of cellular localization | 4.7E−02 |
| negative regulation of cellular process | 4.8E−02 |
| response to DNA damage stimulus | 4.9E−02 |

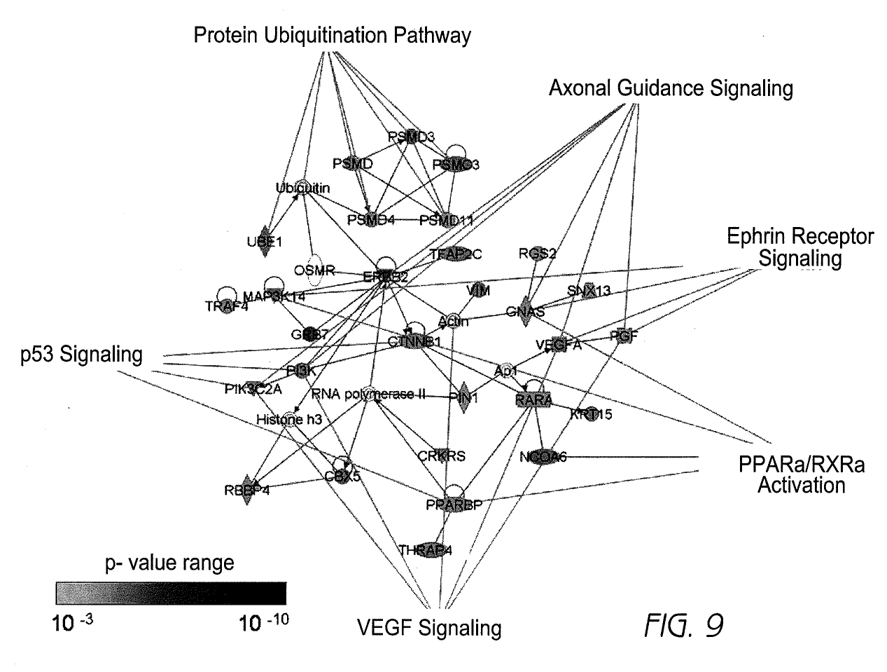

Transmembrane receptor protein tyrosine kinase signaling pathway and intracellular receptor-mediated signaling pathway are among the significant terms, as expected for an inhibitor of ERBB2 and EGFR. Enriched networks and pathways in this gene set were also searched for against the Ingenuity database (http://www.ingenuity.com/). Again, the most significant network had ERBB2 as a major node (FIG. 9a). This network was found to be associated with 5 major signaling pathways: protein ubiquitination, p53 signaling, PPARa/RXRa activation, VEGF signaling and axonal guidance signaling (FIG. 9b). In addition, ephrin receptor signaling pathway also emerged as significant (Table 5).

TABLE 5
The pathways enriched in the Ingenuity analysis of
significant mRNA markers of response to Lapatinib.

| Pathway | p-value |
|---|---|
| Axonal guidance signaling | 1.4E−05 |
| Ephrin receptor signaling | 5.6E−05 |
| Protein ubiquitination | 2.2E−03 |
| PPARa/RXRa activation | 3.0E−03 |
| VEGF signaling | 3.1E−03 |

Numerous novel predictors were identified across all three molecular datasets. For instance, among proteomic predictors, ephrin-A1 (EFNA1) and JAK1 emerged as significant. The association with EFNA1 levels can be explained by the fact that the ERBB2 positive cells are uniformly in the luminal subtype which express higher levels of EFNA1. EFNA1 was also found to be statistically significant at the mRNA level (Table 3a). The negative association with JAK1 protein levels is interesting since JAK1 is encoded in the 1p32 amplicon that has reduced copy number in ERBB2-positive tumors. This suggests that JAK1 or another gene encoded in this amplicon may attenuate response to Lapatinib when amplified.

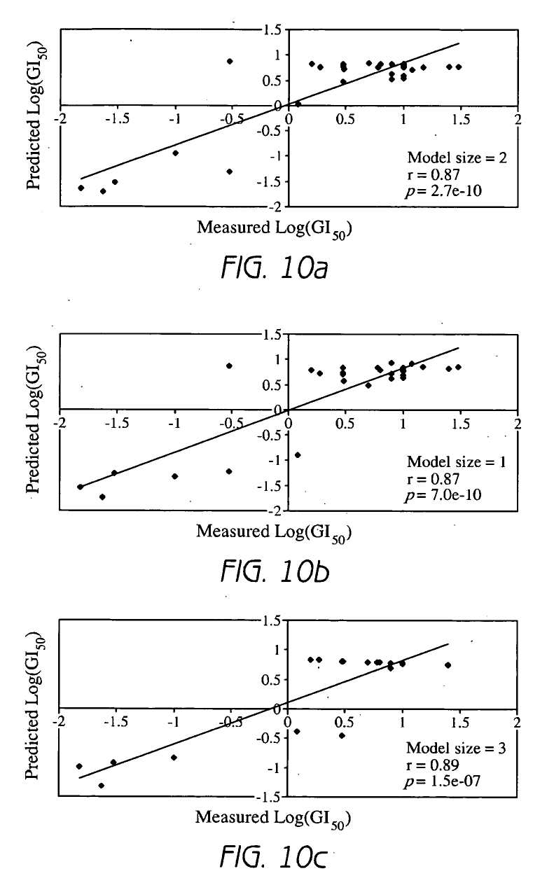

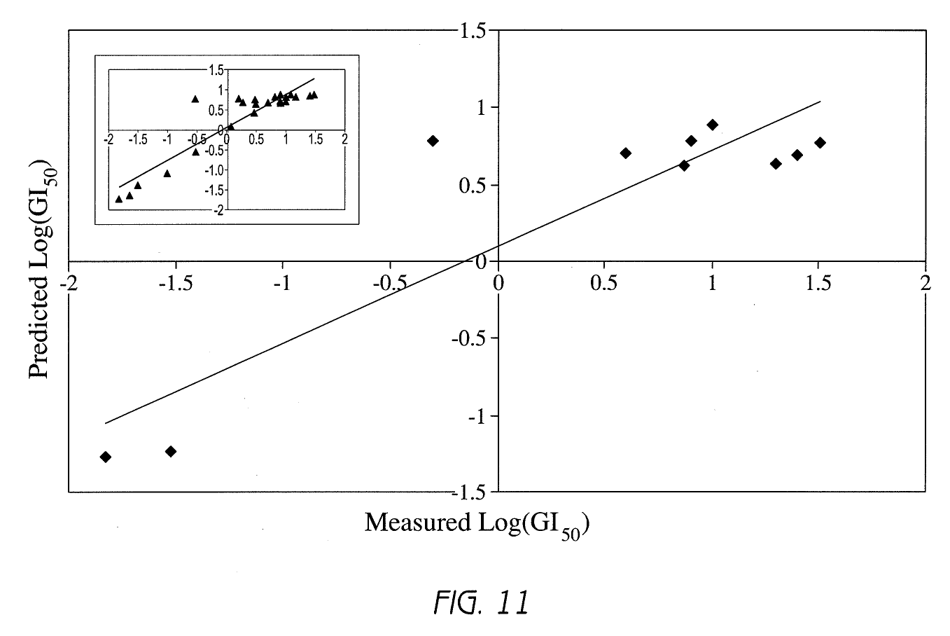

Multivariate models. To obtain a multivariate model that combines inputs from all three molecular datasets, an integrative approach was used. For a multivariate model for a given data-type, the weighted voting method was used, as in Example 2. A challenge in weighted voting approach is how to determine the model size, i.e. the number of terms in the model. Previous implementations have, sometimes, involved subjective choices. Here the model size was selected to minimize the LOOCV error, which leads to a unique model. The procedure is, otherwise, similar to that described in Example 2. The optimal model size emerged to be 2 for mRNA expression profiles, 1 for DNA copy number profiles and 3 for protein expression profiles (FIG. 10). To obtain the final model that integrates all data types, a weighted voting scheme was again used. The inputs here are the multivariate models for each data type, and the weight for each data type is proportional to the average correlation of top N_{G} markers used in the step above. The predictor performs remarkably well on the test set of 10 cell-lines: the predicted GI₅₀ has a Pearson's correlation of 0.90 with the measured GI₅₀ (p=4.7e-4) (FIG. 11).

Unsupervised classification. Hierarchical clustering of mRNA markers already suggested that adaptive linear splines can automatically identify class-like features. Splines can actually also provide a convenient framework for performing unsupervised classification of cancer samples. The key idea is that the adaptively determined knots segregate the cell-lines into multiple classes (FIG. 8b): the group with high GI₅₀ values is assigned to the resistant class (class=1), the group with low GI₅₀ is assigned to the sensitive class (class=−1), while the cell-lines that lie between the two knots are considered to have an indeterminate class (class=0). If there is only one knot, only the cell-line which lies at the knot is assigned a class score of zero, the rest being assigned to a class as above. For a linear fit, i.e. with no knots, all cell-lines are assigned to the indeterminate class.

A class score was enumerated for each cell-line using the weighted voting scheme described above, where predicted classes were used as inputs instead of the predicted GI₅₀. The weighted class score (W_{c}) of each cell-line was used for its final class assignment: W_{c}>0 indicated more votes in favor of the resistant class, and hence, the cell-line was assigned to the resistant class. Similarly, a cell-line with W_{c}<0 was assigned to the sensitive class, and that with W_{c}=0 to an indeterminate class. FIG. 8c shows the class assignments for the cell-lines in the training set. The maximum GI₅₀ of the (predicted) sensitive class is lower than the minimum GI₅₀ of the resistant class, indicating clear separation characteristic of appropriate classification. The average of these two response values at the separatrix can then be used as a threshold for discriminating the resistant and sensitive cells. For this case, the threshold is −0.46 in the log scale. This is significantly different from the average log(GI₅₀) (=0.40), a threshold often used in the supervised classification methods (11). Using the threshold determined above, 10 out of 10 (=100%) test cell-lines were assigned to the correct class (Table 6a). For purposes of comparison, the average GI₅₀ of the training set was also used to assign whether a cell-line is sensitive or resistant. 9/10 (=90%) samples were assigned to the correct class (Table 6a). This performance is clearly better than that of several previously employed methods, and requires fewer predictive markers and smaller sample sizes than those approaches.

TABLE 6
Predictive accuracy of adaptive linear splines on Lapatinib dataset.
(a) Class prediction accuracy using unsupervised classification.
(b) Comparison of various regression methods. In all cases, the model
was trained on the same set of 30 cell-lines and tested on 10 cell-lines
not used in the training set of the breast cancer cell-line panel.
Table 6a.

| | Number cell-lines with | |
|---|---|---|
| GI50 threshold for sensitive | correctly predicted class | Prediction |
| and resistant cell-lines | (Total = 10) | accuracy (%) |
| Unsupervised | 10 | 100 |
| Average GI50 | 9 | 90 |

Table 6b.

| | Pearson's correlation | |
|---|---|---|
| | between measured and | p-value of |
| Alternative multivariate methods | predicted GI50 in test set | correlation |
| Weighted voting | 0.90 | 4.7E−04 |
| Modified weighted voting | 0.89 | 4.8E−04 |
| Multivariate adaptive regression | 0.88 | 7.7E−04 |
| splines (MARS) | | |
| Principal components regression | 0.73 | 1.7E−02 |
| Multivariate linear regression | 0.75 | 1.3E−02 |

Beyond weighted voting. The voting method can be extended such that the weights in the model are learnt from the data at each step, rather than being predetermined by univariate correlation. This is accomplished by using a least squares fit, which also facilitates learning the significant feature variables (molecular markers). The knots of splines are retained as the same as that obtained from the univariate analysis, however. Variable selection is done here in a stepwise manner. The optimal size of the model is determined by minimizing LOOCV error. The coefficients of the model as obtained via least squares fit are then the weights of each predictor. When the trained model was applied to the test data from 10 cell-lines, the predicted GI₅₀ was found to be correlated with the measured GI₅₀ with a Pearson's correlation of 0.89, corresponding to a p-value of 4.8e-4, which is comparable to the result obtained with weighted voting. ERBB2 emerged as significant in all 3 datasets. In addition, the amplicon CTC-329F6 on chr7p22 was also significant in the DNA copy number data set.

Comparison with other methods. The spline-based approach was compared to a few other related methods used previously. Regression approaches were primarily considered in this context, as they can take all data points into account and do not require subjective partitions of the data set into sensitive and resistant classes a priori. Specifically, multivariate adaptive regression splines (MARS), principal components regression and multivariate linear regression were compared to the spline-based approach described above. MARS uses linear splines as basis functions, but employs a greedy search strategy. The model is built using a combination of forward addition and backward elimination search strategies. A prioritized set of candidate markers was used as input to MARS, where prioritization was done at the univariate level using adaptive linear splines. The PCR method was implemented as described in Mariadason, J. M., Arango, D., Shi, Q., Wilson, A. J., Corner, G. A., Nicholas, C., Aranes, M. J., Lesser, M., Schwartz, E. L. & Augenlicht, L. H. (2003) Cancer Res 63, 8791-812. Very briefly, markers were prioritized using linear regression for the respective dataset. Principal component analysis was performed on their corresponding molecular profiles. Linear regression was performed using the derived principal components. Finally, PCR models for various datasets were combined using a linear model.

Comparison of performances for various methods is shown in Table 6b. The spline-based methods clearly outperform the linear methods, similar to the apoptosis dataset described above. For Lapatinib, weighted voting method performs the best.

Clinical applicability of mRNA markers. In order to determine which markers can be used to stratify the tumor patients in clinic, mRNA expression profiles of 118 breast tumors were collected. Many of our univariate mRNA predictors, derived from the cell-line data, are abundantly expressed in the tumor panel (high expression in ≧50% of tumor samples; data not shown). To quantitatively evaluate the clinical relevance of our markers, the spline-based model described above was trained using only those genes that are abundantly expressed in the tumors. The strength of this model was examined using the same train-test strategy via weighted voting method, as described above. The optimal model size from LOOCV again was determined to be 2. The measured and predicted GI₅₀ are well correlated as before: r=0.85 (p=1.7e-03), indicating that this approach can identify clinically applicable markers. To further assess the clinical applicability of the model, we estimated the sensitivity of 118 tumors using the predicted model. Only 15 tumors were identified as sensitive to Lapatinib, using the unsupervised threshold determined above. 10 out of 15 have DNA amplification data available, from which they have been determined as ERBB2 positive. The remaining 5 tumors express ERBB2 mRNA at high levels. One tumor, which was ERBB2 amplified, was predicted as resistant, representing a false negative. However, ERBB2 amplification was much smaller in this sample compared to the others.

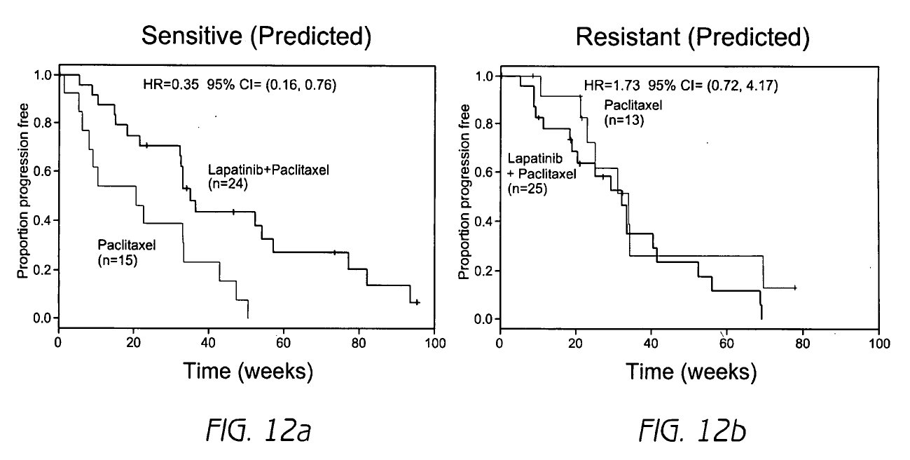

Finally, the ability of a 6 transcript predictor of response to Lapatinib was tested using in vitro measurements. Specifically, the predictor was used to stratify patient response to Lapatinib in the EGF30001 trial of Lapatinib plus Paclitaxel vs. Paclitaxel plus placebo. This predictor was comprised of two genes (ERBB2 and GRB7) for which increased transcription levels were associated with sensitivity in vitro and four genes (CRK, ACOT9, FLJ31079 (CBX5), and DDX5) for which increased transcription levels were associated with resistance in vitro. The progression free survival in 49 ERBB2 positive tumors treated with Lapatinib plus Paclitaxel (L+P) and 28 ERBB2 positive tumors treated with Paclitaxel plus placebo (P) was analyzed (FIG. 12). Among the predicted sensitive patients, the hazards ratios (HR) of L+P vs P was HR=0.35 (95% CI=(0.16, 0.76)). Whereas, among the predicted resistant patients, the corresponding HR=1.73 (95% CI=(0.72, 4.17)). This indicates that the predictor developed in vitro does stratify response in patients.

However, the predictor did not stratify 110 patients with ERBB2 negative tumors treated with Lapatinib plus Paclitaxel or 115 patients with ERBB2 negative treated with Paclitaxel plus placebo (hazards ratios of 1.04 95% CI=(0.67-1.61) and 0.99 95% CI=(0.66-1.47); respectively). These analyses indicate that the in vitro markers of response to Lapatinib can correctly stratify the ErbB2 positive patients into responders and non-responders. Taken together, these results suggest that the adaptive splines based approach can be used to identify the clinically applicable markers.

### Example 4

### Metabolism in Breast Cancer Cells

A spline-based algorithm was used to identify the mRNA markers that are predictive of glycolytic index. Specifically, the baseline mRNA profiles were correlated with the logarithm of glycolytic index values (GIVs) using an adaptive splines framework. In this approach, both magnitude and class-type of response are simultaneously modeled. Although the GIVs were used as input to the algorithm, i.e. without binarization, the method could automatically identify two-class like partition in the data. This is revealed by performing an unsupervised hierarchical clustering of the mRNA expression levels of the top 100 predictors identified by the spline-based algorithm. The 8 cell-lines in the left hand partition have generally high GIVs, while the 5 cell-lines to the right have low GIVs. Only one cell-line (BT549) is misclassified. This clearly indicates that we have been able to identify markers that can discriminate cancer samples with high GIVs from those with low GIVs. Additionally, this demonstrates the power of the spline-based algorithm—in that it could identify markers using only 13 samples, as opposed to previous methods which typically require ˜50-100 samples at least.

### Example 5

### Lapatinib Treatment of 40 Breast Cancer Cell Lines Shows a Wide Range of Quantitative Responses to Treatment

It has been demonstrated that a collection of breast cancer cell-lines can be used as a model of much of the genomic and transcriptional diversity in primary breast tumors. The biological and molecular features of the breast cancer cell lines and cell culture conditions were described in detail in Neve et al. (“A collection of breast cancer cell lines for the study of functionally distinct cancer subtypes”, Cancer Cell 10:515-527, 2006), which is incorporated in its entirety by reference.

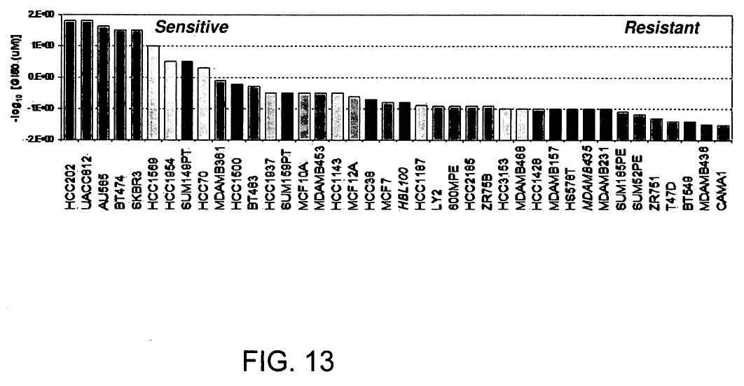

In this example, the responses of 40 breast cancer cell lines to Lapatinib treatment were analyzed and the responses were correlated with genomic, transcriptional and protein profiles of the cell lines to identify molecular features that were associated with the responses. Each cell line was treated in triplicate for 3 days with 9 concentrations of Lapatinib at concentrations ranging from 0.077 nM to 30 μM. The concentration of Lapatinib needed to inhibit growth by 50% (GI₅₀) was calculated for each cell line as described in Monks et al. (“Feasibility of a high-flux anticancer drug screen using a diverse panel of cultured human tumor cell lines”, J. Natl. Cancer Inst. 83:757-766, 1991), which is incorporated in its entirety by reference. The GI₅₀ values ranged from 0.015 μM to ≧30 μM across the collection of cell lines (FIG. 13). This study shows that different breast cancer cell lines show a wide range of quantitative responses to Lapatinib treatment.

### Example 6

### Identification of Molecular Markers Predictive of Response to Lapatinib by Adaptive Splines

The dose response curves for Lapatinib in a panel of 40 breast cancer cell lines were measured using the method of Neve et al. (“A collection of breast cancer cell lines for the study of functionally distinct cancer subtypes”, Cancer Cell 10:515-527, 2006), which is incorporated in its entirety by reference. The response curves were used to estimate the GI₅₀ value for each cell line, which were then used to perform the correlative analyses for sensitivity prediction. To identify the computational model and the predictive markers of sensitivity to Lapatinib, from cell-line panel, a training set of 30 cell-lines were randomly selected, which were used for further to learn the molecular markers and the computational model for sensitivity prediction. The remaining 10 cell-lines were used to test the accuracy of the model.

The computational model is expressed as a sum of linear splines. For this description, a linear spline (x−ξ)₊ is defined as: (x−ξ)₊=x−ξ, for x>ξ, and 0, otherwise. ξ is often referred to as a knot.

The response to 4-anilinoquinazoline kinase inhibitor (e.g., Lapatinib), f(x), predicted by any specific gene, is written in terms of the values, {gₖ}, achieved by the spline function f (x) at the knots {ξₖ} (where x is log₂(expression level of the gene)):

$$f ( x ) = {g}_{0} ( 1 - {\hat{h}}_{1} ) + {\sum }_{j = 1}^{M} {g}_{j} ( {\hat{h}}_{j} - {\hat{h}}_{j + 1} ) + {g}_{M + 1} {\hat{h}}_{M + 1} , \qquad ( 1 )$$

where the model contains M internal knots, ξ₁, . . . ξ_{M}, is written as (ξ₀ and ξ_{M+1} are the values of x at the boundary), ξ₀<ξ₁<K<ξ_{M}ξ_{M+1}, and ĥₖ≡ĥₖ(x) is defined as:

$${\hat{h}}_{k} ( x ) = \frac{{h}_{k} ( x )}{{ξ}_{k} - {ξ}_{k - 1}} \qquad ( 2 )$$

The function hₖ (x) is defined as:

hₖ(x)=(x−ξₖ₋₁)₊−(x−ξₖ)₊ (3)

There is a separate function f(x) for each gene tested.

The complete prediction from all genes is based on the following model:

$$log ( {GI}_{50} ) = {\sum }_{k = 1}^{{N}_{G}} {w}_{n} * log ( {( {GI}_{50} )}_{n} , \qquad ( 4 )$$

where n is an index for the gene id, log(GI₅₀)ⁿ is the predicted value of log(GI₅₀) based on the gene n only (as above, same as the function f (x) ), N_{G} is the total number of genes used, and wₙ indicates the normalized weight for gene n:

$${w}_{n} = log ( {p}_{n} ) / {\sum }_{n = 1}^{{N}_{G}} log ( {p}_{n} ) \qquad ( 5 )$$

where pₙ is the p-value of the univariate fit for the above spline function, f (x), for gene n in the training set of 30 cell-lines. When a subset of genes is used, the model is recomputed with appropriate value of N_{G} and appropriate set of {pₙ}.

Genome-wide correlation of mRNA levels with the measured GI₅₀ values were performed to identify statistically significant mRNA markers (p<5e-03, FDR<5%). The analysis was done twice: once where all cell-lines were included, and the other where only ERBB2-negative cell-lines were used. Next, the intersection of these two gene sets was sought by looking for genes that had same predictive patterns in these two analyses (resistant in both or sensitive in both), and were abundantly expressed in the tumor panel (log₂(expression intensity)≧8 in at least 50% of the tumors). Those genes that were predictive of resistance to Lapatinib were retained and added to this, n=2 genes (ERBB2 and GRB7), which were highly enriched in the tumor panel and had strong predictive power in the entire cell-line panel (n was determined using cross-validation analysis). When the trained model was tested on a set of 10 cell-lines, the predicted and measured sensitivity had a statistically significant Pearson's correlation: r=0.92 (p=1e-4). The genes identified are described in Tables 7a and 7b. The cell lines that were found sensitive to Lapatinib are found in Table 9. The average log₂(expression) of 6 of the identified genes are listed in Table 10.

TABLE 7a
6 genes identified to be predictive of sensitivity status to Lapatinib

| | | | | | | | Predicts |
|---|---|---|---|---|---|---|---|
| | | | | | | | sensitivity |
| | | | | | | | (S) or |
| | | Gene | | 1Linear | | | Resistance |
| ID | Probe ID | symbol | Adaptive Spline | Spline | t_test | Linear_fit | (R) |
| 2064 | 216836_s_at | ERBB2 | 5.82E−10 | 1.52E−11 | 7.32E−05 | 6.63E−07 | S |
| 2886 | 210761_s_at | GRB7 | 6.12E−10 | 1.22E−08 | 6.34E−05 | 7.75E−08 | S |
| AW612311 | 202225_at | CRK | 6.52070E−4 | 0.0159059 | 0.3700049 | 0.2153449 | R |
| 23597 | 221641_s_at | ACOT9 | 8.49190E−4 | 0.0264765 | 0.2522735 | 0.1351097 | R |
| BG391282 | 212126_at | FLJ31079 | 0.0016429 | 0.0054836 | 0.1809480 | 0.3909226 | R |
| | | (CBX5) | | | | | |
| 1655 | 200033_at | DDX5 | 0.0047115 | 0.0060051 | 0.6337171 | 0.2716488 | R |

TABLE 7b
13 genes identified to be predictive of
sensitivity status to Lapatinib

| | | | | Predicts |
|---|---|---|---|---|
| | | | | sensitivity |
| | | | | (S) or |
| | | Gene | Adaptive | Resistance |
| ID | Probe ID | symbol | Spline | (R) |
| 205 | 204348_s_at | AK3L1 | 0.0015803 | S |
| 780 | 208779_x_at | DDR1 | 0.0012922 | S |
| 1356 | 204846_at | CP | 0.0011653 | S |
| 1366 | 202790_at | CLDN7 | 0.0009682 | S |
| 2778 | 214157_at | GNAS | 0.0004605 | S |
| 5268 | 204855_at | SERPINB5 | 0.0015919 | S |
| 8525 | 207556_s_at | DGKZ | 0.0009794 | S |
| 9221 | 211949_s_at | NOLC1 | 0.0010461 | R |
| 23650 | 202504_at | TRIM29 | 0.0010247 | S |
| 23710 | 211458_s_at | GABARAPL1 | 0.0015302 | S |
| 55701 | 58780_s_at | FLJ10357 | 0.0007140 | R |
| 57728 | 220917_s_at | WDR19 | 0.0019107 | R |
| 442871 | 212560_at | SORL1 | 0.0005058 | S |

TABLE 8a
The genome locations of 6 genes identified to be predictive of sensitivity status to Lapatinib

| | | Gene | Chromo. | Gene | Accession | | Genome |
|---|---|---|---|---|---|---|---|
| ID | Probe ID | Symbol | location | ID | No. | Unigene | location |
| 2064 | 216836_s_at | ERBB2 | chr17q11.2-q12\| | 2064 | X03363 | Hs.446352 | chr17: 35109876-35138354 (+) |
| | | | 17q21.1 | | | | // 98.14 // q12 |
| 2886 | 210761_s_at | GRB7 | chr17q12 | 2886 | AB008790 | Hs.86859 | chr17: 35152029-35156782 (+) |
| | | | | | | | // 99.7 // q12 |
| AW612311 | 202225_at | CRK | — | — | AW612311 | Hs.461896 | chr17: 1270736-1306262 (−) |
| | | | | | | | // 84.09 // p13.3 |
| 23597 | 221641_s_at | ACOT9 | chrXp22.11 | 23597 | AF241787 | Hs.298885 | chrX: 23631714-23635045 (−) |
| | | | | | | | // 98.39 // p22.11 |
| BG391282 | 212126_at | FLJ31079 | — | — | BG391282 | Hs.349283 | chr12: 52910994-52913958 (−) |
| | | (CBX5) | | | | | // 85.17 // q13.13 |
| 1655 | 200033_at | DDX5 | chr17q21 | 1655 | NM_004396 | Hs.279806 | chr17: 59926201-59932869 (−) |
| | | | | | | | // 99.57 // q24.1 |

TABLE 8b
The genome location of 13 genes identified to be predictive of sensitivity status to Lapatinib

| | | Gene | Chromo. | Gene | Accession | | Genome |
|---|---|---|---|---|---|---|---|
| ID | Probe ID | Symbol | location | ID | No. | Unigene | location |
| 205 | 204348_s_at | AK3L1 | chr1p31.3 | 205 | NM_013410 | Hs.10862 | chr1: 65386494-65465286 (+) |
| | | | | | | | // 99.0 // p31.3 /// |
| | | | | | | | chr17: 26696478-26698170 (+) |
| | | | | | | | // 97.01 // q11.2 /// |
| | | | | | | | chr12: 31659132-31660784 (−) |
| | | | | | | | // 94.61 // p11.21 |
| 780 | 208779_x_at | DDR1 | chr6p21.3 | 780 | NM_001954 | Hs.631988 | chr6: 30960319-30975908 (+) |
| | | | | | | | // 96.82 // p21.33 /// |
| | | | | | | | chr6_cox_hap1: |
| | | | | | | | 2300945-2316536 (+) |
| | | | | | | | // 96.74 // /// |
| | | | | | | | chr6_qbl_hap2: |
| | | | | | | | 2099274-2114865 (+) |
| | | | | | | | // 96.82 // |
| 1356 | 204846_at | CP | chr3q23-q25 | 1356 | NM_000096 | Hs.558314 | chr3: 150374065-150422269 (−) |
| | | | | | | | // 99.94 // q24 |
| 1366 | 202790_at | CLDN7 | chr17p13 | 1366 | NM_001307 | Hs.513915 | chr17: 7104179-7107236 (−) |
| | | | | | | | // 99.92 // p13.1 |
| 2778 | 214157_at | GNAS | chr20q13.3 | 2778 | NM_000516 | Hs.125898 | chr20: 56850151-56909306 (+) |
| | | | | | | | // 94.01 // q13.32 |
| 5268 | 204855_at | SERPINB5 | chr18q21.3 | 5268 | NM_002639 | Hs.55279 | chr18: 59295198-59323297 (+) |
| | | | | | | | // 86.52 // q21.33 |
| 8525 | 207556_s_at | DGKZ | chr11p11.2 | 8525 | NM_003646 | Hs.502461 | chr11: 46325697-46358680 (+) |
| | | | | | | | // 97.74 // p11.2 /// |
| | | | | | | | chr13: 43440471-43443843 (+) |
| | | | | | | | // 95.64 // q14.11 |
| 9221 | 211949_s_at | NOLC1 | chr10q24.32 | 9221 | NM_004741 | Hs.523238 | chr10: 103901944-103913617 (+) |
| | | | | | | | // 96.56 // q24.32 |
| 23650 | 202504_at | TRIM29 | chr11q22-q23 | 23650 | NM_012101 | Hs.504115 | chr11: 119487204-119514073 (−) |
| | | | | | | | // 99.57 // q23.3 |
| 23710 | 211458_s_at | GABARAPL1 | chr12p13.2 | 23710 | NM_031412 | Hs.524250 | chr12: 10256765-10266966 (+) |
| | | | /// | | | | // 90.82 // p13.2 /// |
| | | | chr15q26.1 | | | | chr15: 88691822-88693673 (−) |
| | | | | | | | // 100.0 // q26.1 |
| 55701 | 58780_s_at | FLJ10357 | chr14q11.2 | 55701 | NM_018071 | Hs.35125 | chr14: 20627176-20627543 (+) |
| | | | | | | | // 78.84 // q11.2 |
| 57728 | 220917_s_at | WDR19 | chr4p14 | 57728 | NM_025132 | Hs.438482 | chr4: 38860543-38963824 (+) |
| | | | | | | | // 99.3 // p14 |
| 442871 | 212560_at | SORL1 | chr11q23.2-q24.2 | 442871 | BC040643 | Hs.368592 | chr11: 121006842-121009681 (+) |
| | | | | | | | // 98.38 // q24.1 |

TABLE 9
Cell lines found sensitive to Lapatinib.

| | | Training sample or | |
|---|---|---|---|
| Cell Line | log₁₀(GI₅₀) | Test sample | Sensitivity |
| UACC812 | −1.82390874 | Training | Yes |
| HCC202 | −1.82390874 | Test | Yes |
| AU565 | −1.63152716 | Training | Yes |
| BT474 | −1.52287874 | Training | Yes |
| SKBR3 | −1.52287874 | Test | Yes |
| HCC1569 | −1 | Training | Yes |
| HCC1954 | −0.52287874 | Training | Yes |
| SUM149PT | −0.52287874 | Training | Yes |
| HCC70 | −0.30102999 | Test | |
| MDAMB361 | 0.07918124 | Training | |
| HCC1500 | 0.20384846 | Training | |
| BT483 | 0.2757719 | Training | |
| SUM159PT | 0.47712125 | Training | |
| MCF10A | 0.47712125 | Training | |
| MDAMB453 | 0.47712125 | Training | |
| HCC1937 | 0.47712125 | Training | |
| HCC1143 | 0.48358729 | Training | |
| MCF12A | 0.60205999 | Test | |
| HCC38 | 0.69897000 | Training | |
| MCF7 | 0.77815125 | Training | |
| HBL100 | 0.80126647 | Training | |
| HCC1187 | 0.86616914 | Test | |
| LY2 | 0.90308998 | Training | |
| 600MPE | 0.90308998 | Training | |
| HCC2185 | 0.90308998 | Training | |
| ZR75B | 0.90308998 | Test | |
| MDAMB435 | 1 | Training | |
| MDAMB231 | 1 | Training | |
| HCC3153 | 1 | Training | |
| MDAMB157 | 1 | Training | |
| MDAMB468 | 1 | Training | |
| HS578T | 1 | Training | |
| HCC1428 | 1 | Test | |
| SUM185PE | 1.07918124 | Training | |
| SUM52PE | 1.17609125 | Training | |
| ZR75.1 | 1.30102999 | Test | |
| T47D | 1.39794000 | Test | |
| BT549 | 1.39984671 | Training | |
| MDAMB436 | 1.47712125 | Training | |
| CAMA1 | 1.50650503 | Test | |

TABLE 10
The average log₂ (expression) of 6 genes predictive of
sensitivity status to Lapatinib.

| | | Average |
|---|---|---|
| Probe Id | Gene Id | log₂ (expression) |
| 200033_at | DDX5 | 10.17793604 |
| 202225_at | CRK | 6.759778196 |
| 210761_s_at | GRB7 | 7.772996706 |
| 212126_at | FLJ31079 | 7.892459235 |
| | (CBX5) | |
| 216836_s_at | ERBB2 | 8.732683588 |
| 221641_s_at | ACOT9 | 7.110479902 |

In Table 10, the average log₂ (expression) of the genes was determined by measuring the expression levels of the genes in 51 cell lines, including the following cell lines: MDAMB415, MDAMB468, MDAMB157, MDAMB134VI, ZR75.1, SUM44PE, HCC1428, MDAMB361, MDAMB436, SUM52PE, HCC202, BT20, BT549, HCC1937, CAMA1, MDAMB453, MCF12A, HCC70, HBL100, SUM225CWN, HCC38, T47D, SUM1315MO2, HCC3153, HCC1569, HCC2157, BT483, MDAMB435, MCF7, HCC1954, HCC1187, SUM149, HCC1143, AU565, SKBR3, MDAMB175VII, HCC1500, ZR75B, SUM159PT, HCC1008, HCC2185, LY2, SUM190PT, 600MPE, MDAMB231, BT474, UACC812, SUM185PE, HS578T, ZR7530, and MCF10A.

Taken together, these results suggest that the computational based approach has identified clinically applicable molecular markers to stratify cancer patients into responders (sensitive) and non-responders (resistant) to Lapatinib treatment.

### Example 7

### Stratification of a Tumor's Response to Lapatinib by in Vitro Gene Predictors

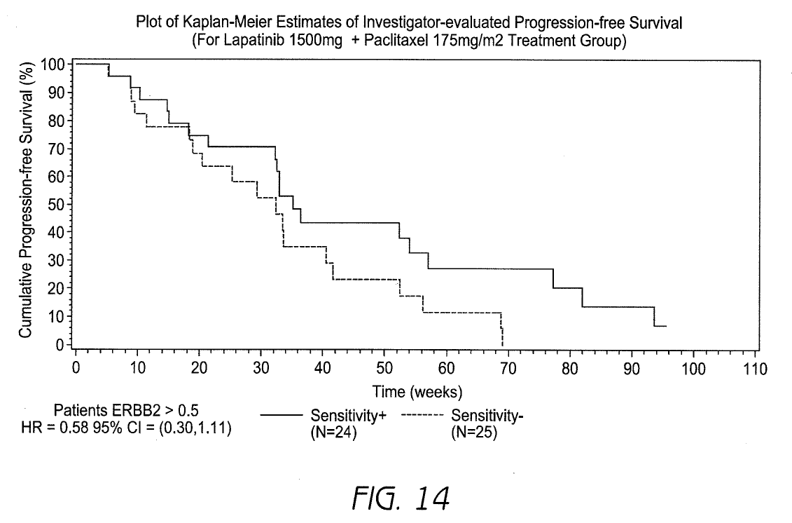

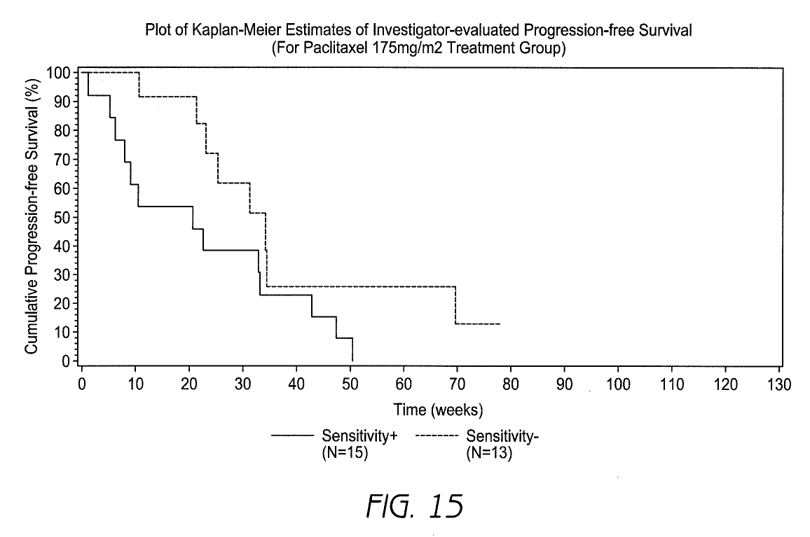

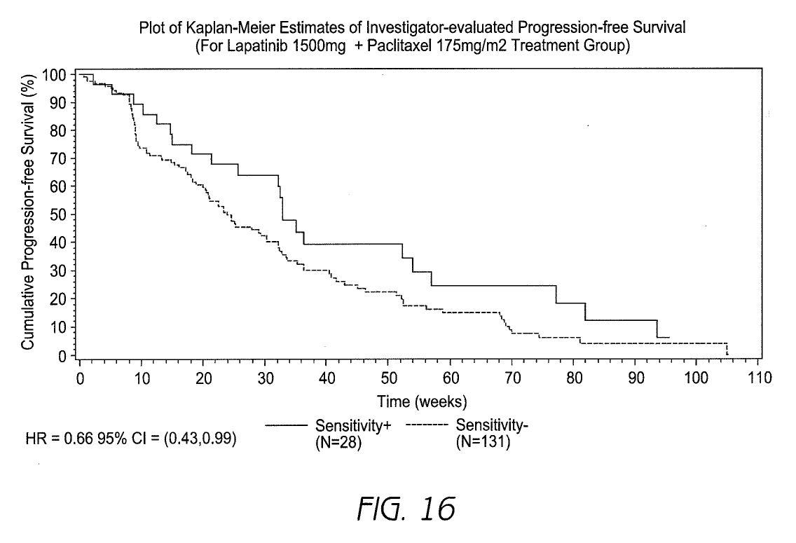

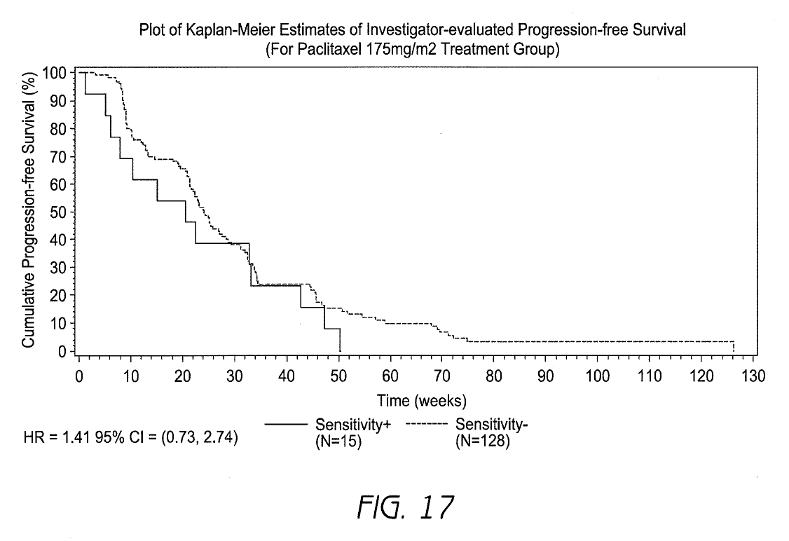

mRNA expression levels of ERBB2, GRB7, CRK, ACOT9, CBX5, and DDX5 genes in a tumor panel from human cancer patients were measured. The computational model described in Example 6 was applied to the mRNA expression levels obtained to predict the Lapatinib sensitivity status of the tumors. The ERBB2-positive tumors (ERBB2 expression level relative to GAPDH ≧0.5, total=78) were stratified as sensitive to Lapatinib if predicted log(GI₅₀)≦0.4 (total=40); others were stratified as resistant to Lapatinib (total=38). The progression free survival of those predicted responders (sensitive) were compared to the non-responders (resistant). It was found that the median survival was longer for the predicted responders who were treated with Lapatinib (FIG. 14), but shorter when treated with placebo (FIG. 15).

This study demonstrates that ERBB2, GRB7, CRK, ACOT9, CBX5, and DDX5 are effective in vitro molecular markers to stratify cancer patients' response to Lapatinib.

### Example 8

### In vitro Gene Predictors Improve Stratification of Patient Response in Two Independent Clinical Trials

The clinical performance of a 6-gene predictor set was retrospectively tested in archival tissue samples from two prospective, randomized clinical trials of Lapatinib monotherapy (EGF20009) and paclitaxel with Lapatinib or placebo (EGF30001). The 6-gene predictor set included ERBB2 and GRB7 genes, whose increased transcription levels were found to be associated with sensitivity to Lapatinib treatment, and CRK, ACOT9, CBX5, and DDX5 genes, whose increased transcription levels were found to be associated with resistance to Lapatinib treatment. Both clinical trials were conducted in patients with newly diagnosed metastatic breast cancer. Quantitative mRNA levels of the transcripts were measured relative to GAPDH using the branch capture (BC) assay from Panomics using RNA extracted from single 10 micrometer FFPE sections from each tumor. Adjacent H&E stained sections were analyzed for tumor content and samples with <50% tumor were excluded. Transcript levels measured using the Panomics BC assay were normalized to Affymetrix microarray equivalent levels using a mapping function developed using measurements of the transcript levels measured in 22 breast cancer cell lines using both platforms. These functions were then applied to Panomics BC transcript levels for tumor samples to obtain Affymetrix-equivalent transcript levels for each of the EERBB2, GRB7, CRK, ACOT9, CBX5, and DDX5 genes. The weights in the 6-gene predictive model for the tumors were the same as determined from cell lines.

### 1. EGF20009 Trial

EGF20009 was a randomized, first line phase II trial in ERBB2-positive patients with advanced or metastatic breast cancer in which patients received Lapatinib as monotherapy. 138 patients with ERBB2-amplified tumors were randomly assigned to one of two Lapatinib dose cohorts: 69 patients received Lapatinib 1,500 mg once daily, and the remaining 69 patients received Lapatinib 500 mg twice daily. Samples from patients treated at both levels of Lapatinib were included in the study and patients were stratified into three groups based on tumor ERBB2 mRNA expression levels measured using the Panomics BC assay. Patients with the highest ERBB2 expression levels were assigned to a group designated as sensitive, patients with the lowest ERBB2 expression levels were assigned to a group designated as resistant and the remaining patients were assigned to an intermediate group (n=53). Patients whose tumors assigned to the intermediate group were further stratified into resistant and sensitive classes by using the 6-gene predictor set and a single response predictor CBX5, respectively.

Stratification of Patient Response to using 6-Predictor Set

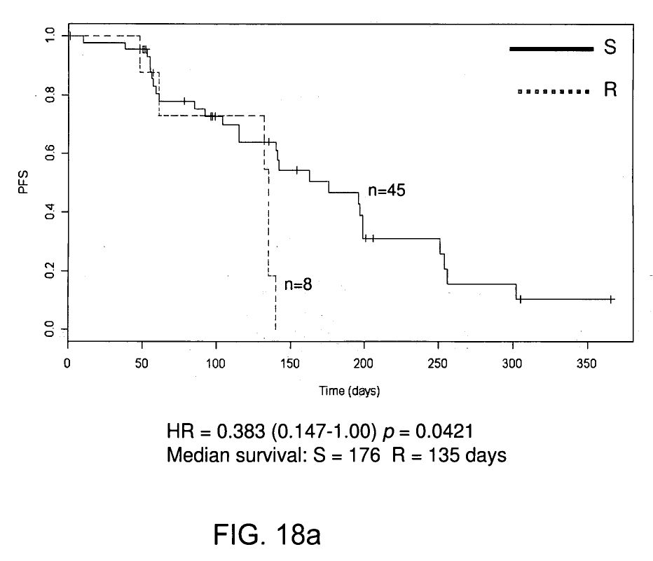

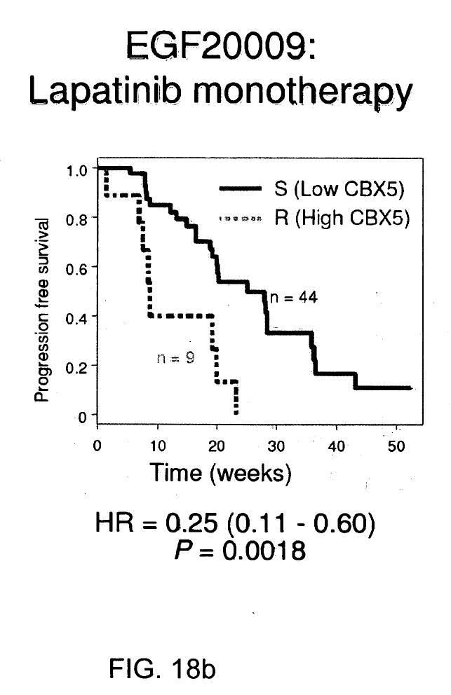

The Kaplan-Meyer plots of progression free survival showed that the 6-gene predictor set stratified 53 patients in the intermediate group into 45 patients predicted to be sensitive compared to 8 patients predicted to be resistant (FIG. 18a). The median survival was longer for the patients predicted to be sensitive, but shorter for the patients to be resistant. The hazards ration (HR) for patients predicted to be sensitive compared to patients predicted to be resistant was 0.383 (95% CI=0.147-1.00; p=0.0421).

CBX as the Single Response Predictor

Using CBX as the single response predictor, 44 patients were predicted to be sensitive to Lapatinib compared to 9 patients predicted to be resistant (FIG. 18b). The median survival was 176 days for patients predicted to be sensitive to Lapatinib, while the median survival was only 61 days for patients predicted to be resistant to Lapatinib. Response rates were 56% and 17% in the patients predicted to be sensitive and resistant, respectively (p=0.02). The hazards ratio (HR) for patients predicted to be sensitive compared to patients predicted to be resistant was 0.25 (95% CI=0.11-0.60; p=0.0018). This result suggests that CBX5 alone is sufficient to predict the sensitivity status of an ERBB2-positive patient to Lapatinib treatment.

### 2. EGF30001 Trial

EGF30001 was a randomized, first-line phase III trial of a combination therapy of paclitaxel plus Lapatinib vs. a therapy of paclitaxel plus placebo for patients with metastatic breast cancer. Patients were randomized assigned to receive one of the two treatments: 291 patients were treated with paclitaxel (175 mg/m² administered every three weeks) plus Lapatinib (1500 mg administered daily), and 288 patients were treated with paclitaxel (175 mg/m² administered every three weeks) plus placebo. Patients with ERBB2-positive and ERBB2-negative tumors were included in the trial although it was intended to be only for patients with ERBB2-negative tumors. As a result, this study included 49 patients with ERBB2-positive tumors that were treated with Lapatinib plus paclitaxel and 28 patients with ERBB2-positive tumors treated with paclitaxel plus placebo.

Stratification of Patient Response to using 6-Gene Predictor set

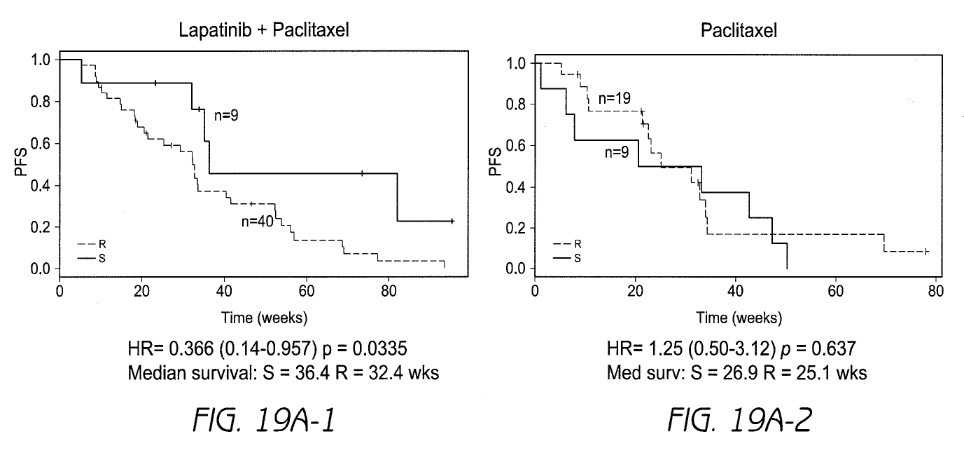

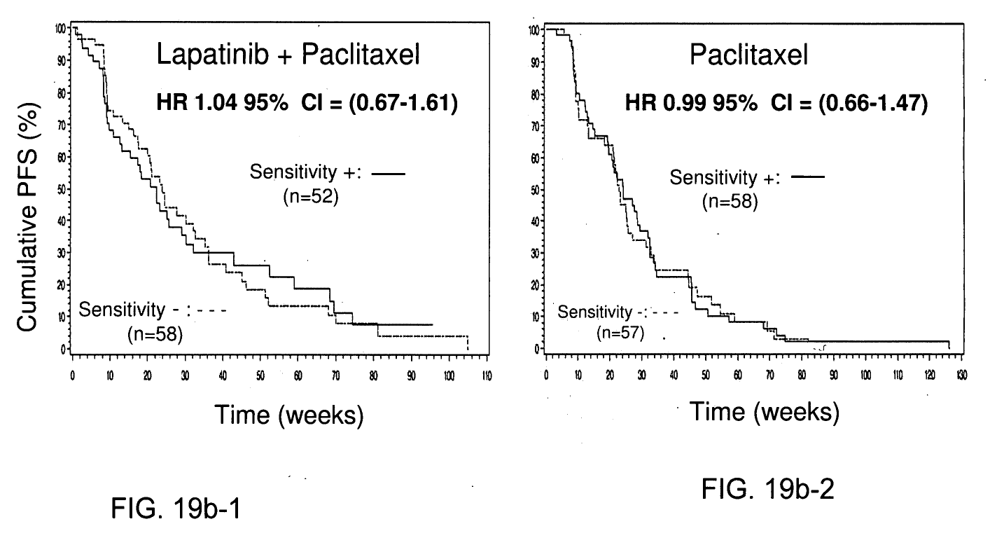

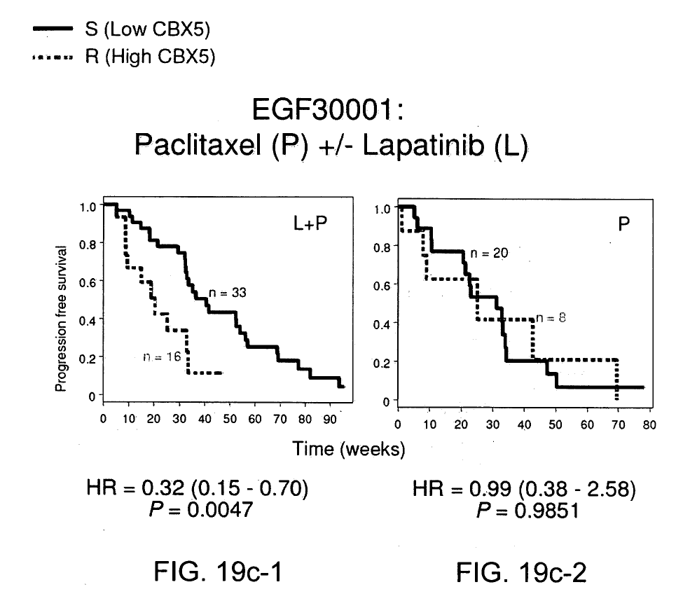

The 6-gene predictor set was also useful in predicting clinical benefit from Lapatinib in combination with paclitaxel in patients with ERBB2-positive tumors (FIG. 19a-1). For the patients treated with Lapatinib in combination of paclitaxel, the median survival was found to be longer for the patients predicted to be sensitive to Lapatinib than the patients predicted to be resistant (HR=0.366, 95% CI=0.14-0.957, p=0.0335). On the other hand, the 6-gene predictor assay did not stratify the 110 patients with ERBB2-negative tumors treated with paclitaxel plus Lapatinib (FIG. 19b-1) or the 115 patients with ERBB2 negative tumors treated with paclitaxel plus placebo (FIG. 19b-2) (HRs are 1.04 (95% CI=0.67-1.61) and 0.99 (95% CI=0.66-1.47), respectively).

CBX as the Single Response Predictor

Using CBX gene as a single-response predictor, for the group of patients treated with paclitaxel plus Lapatinib, the median survival was 40.6 weeks for patients predicted to be sensitive to Lapatinib; while the median survival was only 20.4 weeks for patients predicted to be resistant to Lapatinib (FIG. 19c-1). The hazards ratio (HR) for patients predicted to be sensitive compared to patients predicted to be resistant was 0.32 (95% CI=0.15-0.7; p=0.0047). For the control group of patients treated with paclitaxel plus placebo, the median survival was 31.1 weeks for patients predicted to be sensitive to Lapatinib, while the median survival was 25.1 weeks for patients predicted to be resistant to Lapatinib (FIG. 19c-2). The hazards ratio (HR) for patients predicted to be sensitive compared to patients predicted to be resistant was 0.99 (95% CI=0.38-2.58; p=0.985).

The studies conducted in clinical trials EGF20009 and EGF30001 show that ERBB2, GRB7, CRK, ACOT9, CBX5, and DDX5 genes can be used as in vitro molecular marker to predict patient response to Lapatinib and stratify ERBB2-positive patients into responders (sensitive) and non-responders (resistant). In particular, the CBX5 gene alone was sufficient to predict the sensitivity status of ERBB2-positive breast cancer patients to Lapatinib treatment.

### Example 9

### Evaluation of CBX5 as a Single-Gene Predictor in Stratifying Patient Response to Lapatinib in Clinical Trial EGF100151

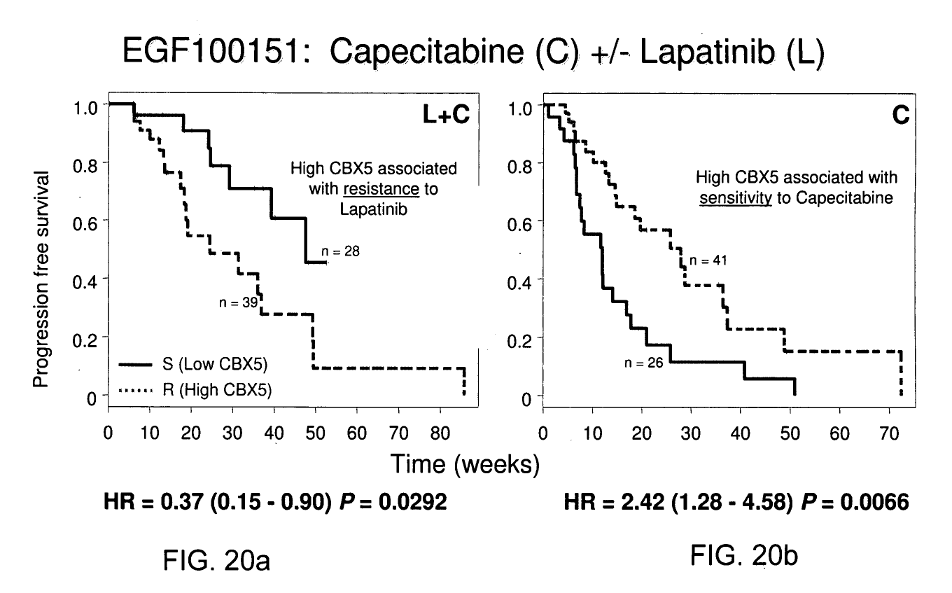

EGF100151 was a randomized, phase III trial in ERBB2-positive patients with incurable stage III or IV of breast cancer who had received prior treatment with anthracyclines, taxanes and trastuzumab. 134 ERBB2-positive patients were randomized to assign to treatment with capecitabine (2000 mg/m² administered every three weeks) plus Lapatinib (1250 mg administered daily) or capecitabine (2500 mg/m² administered every three weeks) plus placebo. Patients were stratified into resistant and sensitive classes to Lapatinib treatment using CBX5 as a single-gene predictor. For patients treated with capecitabine plus Lapatinib, the median survival was found to be longer for patients predicted to be sensitive to Lapatinib than for patients predicted to be resistant to Lapatinib (FIG. 20a). The HR for progression free survival in the 28 patients predicted to be sensitive to Lapatinib treatment vs. 39 patients predicted to be resistant was 0.37 (95% CI=0.15-0.90; p=0.0292).

These results demonstrate that CBX5 alone is sufficient to act as a Lapatinib response predictor.

### Example 10

### Identification of ERBB2-Positive Cancer Patients

A sample, such as blood, cell, tissue or tumor, is obtained from a cancer patient for analysis. The sample is taken from the patient using a common procedure known by persons skilled in the art, including needle biopsy, surgical biopsy, bone marrow biopsy, skin biopsy, or endoscopic biopsy. Blood drawn from the patient also can be analyzed using similar procedures.

The expression level of ERBB2 gene in the patient sample is measured using the Panomics branch capture (BC) assay (Quantigene protocol). The sample obtained from the patient is first processed using Panomics QuantiGene® 2.0 Sample Processing Kit to prepare FFPE tissue homogenates. Then, total RNA is extracted from one 10 μm FFPE section from the sample using a solubilization solution and proteinase K. Centrifugation is performed to purify solubilized RNA from cellular debris and paraffin resulting in ˜250 μl of sample. 3 μl of this sample is used to measure expression level of ERBB2 gene. mRNA for ERBB2 gene is captured in a 96-well microtiter plate using oligonucleotides that bound the mRNA to the capture plate and also provided a oligonucleotide structure for binding of signaling amplification and labeling probes. Gene expression value is measured using a luminescent substrate that is activated upon binding to the label probes hybridized to the target mRNA.

The ERBB2 expression level is then compared with the expression level of the gene encoding ERBB2 in a normal tissue sample or a reference expression level (such as the average expression level of the ERBB2 gene in a cell line panel, a cancer cell, a tumor panel, or the like). An increase in the expression level of ERBB2 in the sample obtained from the cancer patient, as compared to the expression level of ERBB2 in the normal tissue sample or the reference expression level indicates the cancer patient is ERBB2-positive and is suitable for treatment with the 4-anilinoquinazoline kinase inhibitor.

### Example 11

### Identification of a Cancer Patient Suitable for Treatment with a 4-Anilinoquinazoline Kinase Inhibitor by Measuring the Expression Level of GRB7, CRK, ACOT9, CBX5 or DDX5 in the Patient

A sample, such as blood, cell, tissue or tumor, is taken from a cancer patient. The sample is taken from the patient using a common procedure known by persons skilled in the art, such as needle biopsy, surgical biopsy, bone marrow biopsy, skin biopsy, or endoscopic biopsy. Blood drawn from the patient also can be analyzed using similar procedures.

Six genes described in Table 7a, ERBB2, GRB7, CRK, ACOT9, CBX5, and DDX5, are included in this assay. The expression level of those 6 genes in the patient sample is measured using the Panomics branch capture (BC) assay (Quantigene protocol). The sample obtained from the patient is first processed using Panomics QuantiGene® 2.0 Sample Processing Kit to prepare FFPE tissue homogenates. Then, total RNA is extracted from one 10 μm FFPE section from the sample using a solubilization solution and proteinase K. Centrifugation is performed to purify solubilized RNA from cellular debris and paraffin resulting in ˜250 μl of sample. 3 μl of this sample is used to measure expression level of each of the 6 genes. mRNA for each gene is captured in a 96-well microtiter plate using oligonucleotides that bound the mRNA to the capture plate and also provided a oligonucleotide structure for binding of signaling amplification and labeling probes. Gene expression value is measured using a luminescent substrate that is activated upon binding to the label probes hybridized to the target mRNA.

The expression level of each of those 6 genes in the patient sample is compared with the expression level of the respective gene in a normal tissue sample or a reference expression level (such as the average expression level of the gene in a cell line panel, a cancer, a tumor panel, or the like). An increase in the expression level of GRB7 in the patient sample, as compared to the expression level of GRB7 in the normal tissue sample or the reference expression level, indicates the patient, from whom the sample is obtained, is suitable for treatment with a 4-anilinoquinazoline kinase inhibitor. A decrease in the expression of one or more of CRK, ACO79, CBX5, or DDX5, as compared to the expression level of the each gene in the normal tissue sample or the reference expression level, indicates the patient, from whom the sample is obtained, is suitable for treatment with the 4-anilinoquinazoline kinase inhibitor.

### Example 12

### Identification of a Cancer Patient Suitable for Treatment with a 4-Anilinoquinazoline Kinase Inhibitor by Measuring the Expression Level of 13 Molecular Markers in the Patient

A sample, such as cell, tissue or tumor, is taken from a cancer patient. The sample is taken from the patient using a common procedure known by persons skilled in the art, such as needle biopsy, surgical biopsy, bone marrow biopsy, skin biopsy, or endoscopic biopsy. Blood drawn from the patient also can be analyzed using similar procedures.

13 genes described in Table 7b, AK3L1, DDR1, CP, CLDN7, GNAS, SERPINB5, DGKZ, NOLC1, TRIM29, GABARAPL1, FLJ10357, WDR19, and SORL1, are included in this assay. The expression level of each of those 13 genes in the patient sample is measured using the Panomics branch capture (BC) assay (Quantigene protocol). The sample obtained from the patient is first processed using Panomics QuantiGene® 2.0 Sample Processing Kit to prepare FFPE tissue homogenates. Then, total RNA is extracted from one 10 μm FFPE section from the sample using a solubilization solution and proteinase K. Centrifugation is performed to purify solubilized RNA from cellular debris and paraffin resulting in ˜250 μl of sample. 3 μl of this sample is used to measure expression level of each gene. mRNA for each gene is captured in a 96-well microtiter plate using oligonucleotides that bound the mRNA to the capture plate and also provided a oligonucleotide structure for binding of signaling amplification and labeling probes. Gene expression value is measured using a luminescent substrate that is activated upon binding to the label probes hybridized to the target mRNA.

The expression level of each of the 13 genes in the patient sample is compared with the expression level of the respective gene in a normal tissue sample or a reference expression level (such as the average expression level of the gene in a cell line panel or a cancer or tumor panel, or the like). An increase in the expression level of AK3L1, DDR1, CP, CLDN7, GNAS, SERPINB5, DGKZ, TRIM29, GABARAPL1, and SORL1 in the patient sample, as compared to the expression level of each gene in the normal tissue sample or the reference expression level, indicates the patient, from whom the sample is obtained, is suitable for treatment with a 4-anilinoquinazoline kinase inhibitor. A decrease in the gene expression of one or more of NOLC1, FLJ10357, and WDR19, as compared to the expression level of the each gene in the normal tissue sample or the reference expression level, indicates the patient, from who the sample is obtained, is suitable for treatment with the 4-anilinoquinazoline kinase inhibitor.

### Example 13

### Identification of a Cancer Patient Suitable for Treatment with a 4-Anilinoquinazoline Kinase Inhibitor by Measuring the Expression Level of CBX5 Gene in the Patient

A sample, such as cell, tissue or tumor, is obtained from a cancer patient. The sample is taken from the patient using a common procedure known by persons skilled in the art, such as needle biopsy, surgical biopsy, bone marrow biopsy, skin biopsy, or endoscopic biopsy. Blood drawn from the patient also can be analyzed using similar procedures.

The expression level of CBX5 gene in the patient sample is measured using the Panomics branch capture (BC) assay (Quantigene protocol). The sample obtained from the patient is first processed using Panomics QuantiGene® 2.0 Sample Processing Kit to prepare FFPE tissue homogenates. Then, total RNA isxtracted from one 10 μm FFPE section from the sample using a solubilization solution and proteinase K. Centrifugation is performed to purify solubilized RNA from cellular debris and paraffin resulting in ˜250 μl of sample. 3 μl of this sample is used to measure expression level of CBX5. mRNA for CBX5 gene isaptured in a 96-well microtiter plate using oligonucleotides that bound the mRNA to the capture plate and also provided a oligonucleotide structure for binding of signaling amplification and labeling probes. Gene expression value is measured using a luminescent substrate that is activated upon binding to the label probes hybridized to the target mRNA.

The expression level of CBX5 gene in the patient sample is compared with the expression level of CBX5 gene in a normal tissue sample or a reference expression level (such as the average expression level of CBX5 gene in a cell line panel or a cancer or tumor panel, or the like). A decrease in the gene expression of CBX5, as compared to the expression level of CBX5 gene in a normal tissue sample or a reference expression level indicates the patient, from whom the sample is obtained, is suitable for treatment with the 4-anilinoquinazoline kinase inhibitor.

While the above detailed description has shown, described, and pointed out novel features as applied to various embodiments, it will be understood that various omissions, substitutions, and changes in the form and details of the device or process illustrated may be made without departing from that which has been disclosed. As will be recognized, the present invention may be embodied within a form that does not provide all of the features and benefits set forth herein, as some features may be used or practiced separately from others.

What is claimed is:

1. A method for predicting a physiological response of a biological sample to a treatment, the method comprising:
providing a sample physiological response for each of a plurality of training samples to the treatment;
providing a quantification value of a marker for each of the plurality of training samples;
determining a predictive model relating the sample physiological responses to the quantification values, the model comprising a spline function; and
predicting a physiological response of a biological sample to the treatment using the model.

2. The method of claim 1, wherein said quantification value comprises at least one of a protein expression value, an mRNA expression value and a DNA amplification value.

3. The method of claim 1, further comprising predicting a patient physiological response of a patient based on the predicted physiological response, wherein said biological sample was obtained from said patient.

4. The method of claim 1, further comprising:
providing a quantification value for each of a plurality of markers for each of the plurality of training samples; and
determining a plurality of models relating the sample physiological responses to the quantification values of each of the markers, each model comprising a spline function.

5. The method of claim 4, wherein the number of the plurality of markers is greater than the number of the plurality of training samples.

6. The method of claim 4, wherein the plurality of markers comprise at least about 100 markers.

7. The method of claim 4, wherein the plurality of markers comprise at least about 1000 markers.

8. The method of claim 4, further comprising identifying significant markers, the significant markers having values that are predictive of the sample physiological response.

9. The method of claim 4, further comprising determining a multivariate model based on the plurality of models.

10. The method of claim 9, wherein the multivariate model is determined using a weighted averaging process.

11. The method of claim 4, further comprising:
determining a plurality of multivariate models, each of the multivariate models being based on the plurality of models; and
integrating the multivariate models into a single model.

12. The method of claim 1, wherein the sample physiological response comprises a number.

13. The method of claim 1, wherein the sample physiological response comprises a value related to cell viability.

14. The method of claim 1, wherein the sample physiological response comprises a classification.

15. The method of claim 14, wherein the classification indicates whether the sample is resistant or sensitive to the treatment.

16. The method of claim 14, wherein said classification is determined based on a knot of the spline function.

17. The method of claim 1, wherein said spline function comprises a linear spline function.

18. The method of claim 1, wherein said spline function comprises a polynomial spline.

19. The method of claim 1, wherein said spline function comprises an adaptive spline function.

20. The method of claim 1, wherein said determining a predictive model comprises determining the number and location of zero or more knots in said spline function; and subsequently determining additional spline parameters using a cross-validation error function.

21. A system for relating quantification values of markers to physiological response, the system comprising:
an input component configured to receive input data for each of a plurality of samples, the input data comprising a physiological response to a treatment and a quantification value of a marker in the sample;
a univariate model generator configured to determine a univariate model relating the physiological response to the quantification value using a spline-based analysis; and
an output device configured to output one or more variables or equations related to the univariate model.

22. The system of claim 21, wherein said spline-based analysis comprises an adaptive spline-based analysis.

23. The system of claim 21, wherein said quantification value comprises at least one of a protein expression value, an mRNA expression value and a DNA amplification value.

24. The system of claim 21, wherein said spline-based analysis comprises fitting a linear, adaptive spline to data relating the physiological response to the quantification value.

25. The system of claim 21, further comprising a marker clustering component configured to cluster markers by a clustering method using the univariate models.

26. The system of claim 21, further comprising a multivariate model generator configured to determine a multivariate model relating the physiological response to quantification values of the plurality of markers using a plurality of univariate models,
wherein input data comprises values of a plurality of markers in the sample, and
wherein said univariate model generator is configured to determine a plurality of univariate models, each model being associated with one of the plurality of markers.

27. The system of claim 21, further comprising a physiological response predictor configured to determine a physiological prediction based on the univariate model.

28. The system of claim 21, wherein the input device comprises at least one of a keyboard, a mouse, or a memory storage device

29. The system of claim 21, wherein the output comprises a printer or a display.

30. The system of claim 21, wherein the one or more variables comprises a classification.

31. The system of claim 21, wherein the one or more variables comprise coefficients from the univariate model.

32. The system of claim 21, wherein the one or more variables comprise a multivariate model based on the univariate model or at least one of coefficients, significance and fit values associated with the multivariate model.

33. The system of claim 21, wherein said system comprises a central processing unit (CPU) and a memory.

34. A method for identifying a marker influencing a physiological response of a sample, the method comprising:
providing a physiological response for each of a plurality of training samples to the treatment;
providing a value of each of a plurality of markers for each of the plurality of training samples;
determining a plurality of univariate models, each model relating the physiological responses to values of one of the plurality the marker, each model comprising a spline function; and
identifying a marker influencing the physiological response based on the plurality of univariate models.

35. The method of claim 34, wherein the identifying a marker comprises a clustering process.

36. The method of claim 34, wherein said quantification value comprises at least one of a protein expression value, an mRNA expression value and a DNA amplification value.

37. The method of claim 34, wherein said spline function comprises a linear spline.

38. The method of claim 34, wherein said spline function comprises an adaptive spline.

39. The method of claim 34, further comprising predicting the physiological response of a testing sample based on a value of the identified marker.

40. The method of claim 34, wherein said determining a plurality of univariate models comprises determining the number and location of zero or more knots in said spline function of each model and subsequently determining additional spline parameters using a cross-validation error function.

41. A method for determining if a cancer patient is suitable for treatment with a 4-anilinoquinazoline kinase inhibitor, comprising:
measuring the expression level of one or more genes selected from the group consisting of the genes encoding GRB7, CRK, ACOT9, CBX5, and DDX5 in a biological sample from the cancer patient; and
comparing the expression level of the one or more genes to the expression level of the one ore more genes from a patent without cancer, wherein an increase in the expression level of GRB7, or a decrease in the expression level of one or more genes encoding CRK, ACOT9, CBX5, and DDX5 indicates the patient is suitable for treatment with the 4-anilinoquinazoline kinase inhibitor.

42. The method of claim 41, further comprising:
measuring the expression level of a gene encoding ERBB2 in a sample from the patient; and
comparing the expression level of the gene encoding ERBB2 and the expression level of the gene encoding ERBB2 in the patient without cancer, wherein an increase in the expression level of ERBB2 indicates the patient is suitable for treatment with the 4-anilinoquinazoline kinase inhibitor.

43. The method of claim 41, wherein the expression level of two or more genes selected from the group consisting of the genes encoding GRB7, CRK, ACOT9, CBX5, and DDX5 in a sample from the patient is measured.

44. The method of claim 43, wherein the expression level of three or more genes selected from the group consisting of the genes encoding GRB7, CRK, ACOT9, CBX5, and DDX5 in a sample from the patient is measured.

45. The method of claim 44, wherein the expression level of four or more genes selected from the group consisting of the genes encoding GRB7, CRK, ACOT9, CBX5, and DDX5 in a sample from the patient is measured.

46. The method of claim 45, wherein the expression level of the genes encoding GRB7, CRK, ACOT9, CBX5, and DDX5 in a sample from the patient is measured.

47. The method of claim 41, wherein the cancer is breast cancer.

48. A method for identifying a cancer patient suitable for treatment with a 4-anilinoquinazoline kinase inhibitor, comprising:
measuring the expression level of a gene encoding CBX5 in a sample obtained from the cancer patient; and
comparing the expression level of the gene encoding CBX5 from the cancer patient with the expression level of the gene encoding CBX5 in a patient without cancer, wherein a decrease of expression of the gene encoding CBX5 indicates the patient is sensitive to the 4- anilinoquinazoline kinase inhibitor.

49. The method of claim 48, wherein the patient is an ERBB2-positive patient.

50. A method for identifying a cancer patient suitable for treatment with a 4-anilinoquinazoline kinase inhibitor, comprising:
measuring the expression level of one or more genes selected from the group consisting of the genes encoding AK3L1, DDR1, CP, CLDN7, GNAS, SERPINB5, DGKZ, NOLC1, TRIM29, GABARAPL1, FLJ10357, WDR19, and SORL1 in a sample obtained from the cancer patient; and
comparing the expression level of said gene from the cancer patient with the expression level of the gene in from a patient without cancer wherein an increase in the expression level of one gene selected from the group consisting of the genes encoding AK3L1, DDR1, CP, CLDN7, GNAS, SERPINB5, DGKZ, TRIM29, GABARAPL1, and SORL1, or a decrease of expression of one gene selected from the group consisting of the genes encoding NOLC1, FLJ10357, and WDR19 indicates the patient is sensitive to the 4-anilinoquinazoline kinase inhibitor.


SEQUENCE LISTING (44 sequences)

SEQ ID NO: 1 - DNA, 4624 bp, Homo sapiens
ggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 2 - DNA, 2260 bp, Homo sapiens
ttttagtttccttgggcctggaatctggacacacagggctcccccccgcctctgacttctctgtccgaagtcgggacaccctcctaccacctgtagagaagcgggagtggatctgaaataaaatccaggaatctgggggttcctagacggagccagacttcggaacgggtgtcctgctactcctgctggggctcctccaggacaagggcacacaactggttccgttaagcccctctcttgctcagacgccatggagctggatctgtctccacctcatcttagcagctctccggaagacctttgcccagcccctgggacccctcctgggactccccggccccctgatacccctctgcctgaggaggtaaagaggtcccagcctctcctcatcccaaccaccggcaggaaacttcgagaggaggagaggcgtgccacctccctcccctctatccccaaccccttccctgagctctgcagtcctccctcacagagcccaattctcgggggcccctccagtgcaagggggctgctcccccgcgatgccagccgcccccatgtagtaaaggtgtacagtgaggatggggcctgcaggtctgtggaggtggcagcaggtgccacagctcgccacgtgtgtgaaatgctggtgcagcgagctcacgccttgagcgacgagacctgggggctggtggagtgccacccccacctagcactggagcggggtttggaggaccacgagtccgtggtggaagtgcaggctgcctggcccgtgggcggagatagccgcttcgtcttccggaaaaacttcgccaagtacgaactgttcaagagctccccacactccctgttcccagaaaaaatggtctccagctgtctcgatgcacacactggtatatcccatgaagacctcatccagaacttcctgaatgctggcagctttcctgagatccagggctttctgcagctgcggggttcaggacggaagctttggaaacgctttttctgcttcttgcgccgatctggcctctattactccaccaagggcacctctaaggatccgaggcacctgcagtacgtggcagatgtgaacgagtccaacgtgtacgtggtgacgcagggccgcaagctctacgggatgcccactgacttcggtttctgtgtcaagcccaacaagcttcgaaatggccacaaggggcttcggatcttctgcagtgaagatgagcagagccgcacctgctggctggctgccttccgcctcttcaagtacggggtgcagctgtacaagaattaccagcaggcacagtctcgccatctgcatccatcttgtttgggctccccacccttgagaagtgcctcagataataccctggtggccatggacttctctggccatgctgggcgtgtcattgagaacccccgggaggctctgagtgtggccctggaggaggcccaggcctggaggaagaagacaaaccaccgcctcagcctgcccatgccagcctccggcacgagcctcagtgcagccatccaccgcacccaactctggttccacgggcgcatttcccgtgaggagagccagcggcttattggacagcagggcttggtagacggcctgttcctggtccgggagagtcagcggaacccccagggctttgtcctctctttgtgccacctgcagaaagtgaagcattatctcatcctgccgagcgaggaggagggccgcctgtacttcagcatggatgatggccagacccgcttcactgacctgctgcagctcgtggagttccaccagctgaaccgcggcatcctgccgtgcttgctgcgccattgctgcacgcgggtggccctctgaccaggccgtggactggctcatgcctcagcccgccttcaggctgcccgccgcccctccacccatccagtggactctggggcgcggccacaggggacgggatgaggagcgggagggttccgccactccagttttctcctctgcttctttgcctccctcagatagaaaacagcccccactccagtccactcctgacccctctcctcaagggaaggccttgggtggccccctctccttctcctagctctggaggtgctgctctagggcagggaattatgggagaagtgggggcagcccaggcggtttcacgccccacactttgtacagaccgagaggccagttgatctgctctgttttatactagtgacaataaagattattttttgatacaaaaaaaaaaaaaaaaaaaaaaaa

SEQ ID NO: 3 - DNA, 2245 bp, Homo sapiens
cccgcggctgccgccgccatttcgggcgctgctgtgaagctgaaaccggagccggtccgctgggcggcgggcgccgggggccggaggggcgcgcgcggcggcggcaccccagcgtttaggcgcggaggcagccatggcgggcaacttcgactcggaggagcggagtagctggtactgggggaggttgagtcggcaggaggcggtggcgctgctgcagggccagcggcacggggtgttcctggtgcgggactcgagcaccagccccggggactatgtgctcagcgtctcagagaactcgcgcgtctcccactacatcatcaacagcagcggcccgcgcccgccggtgccaccgtcgcccgcccagcctccgcccggggtgagcccctccagactccgaataggagatcaagagtttgattcattgcctgctttactggaattctacaaaatacactatttggacactacaacgttgatagaaccagtttccagatccaggcagggtagtggagtgattctcaggcaggaggaggcggagtatgtgcgagccctctttgactttaatgggaatgatgaggaagatcttccctttaagaaaggagacatcttgagaatccgggacaagcctgaagagcagtggtggaatgcggaggacagcgaaggcaagagagggatgattccagtcccttacgtcgagaagtatagacctgcctccgcctcagtatcggctctgattggaggtcggtgagctggtaaaggttacgaagattaatgtgagtggtcagtgggaaggggagtgtaatggcaaacgaggtcacttcccattcacacatgtccgtctgctggatcaacagaatcccgatgaggacttcagctgagtatagttcaacagttttgctgacagatgggaacaatcttttttttttttttccaactgccatctatacaattttcttacagatgtcaaaagcagtctagtttatataagcattctgttacctgtgatattttttagactgaactgctccattcctagtcttaattaccatattcagggtacgaactggagggcttgtgtgttagcttctgaattggcaattggaggcggtagtggtcgtgcctgtgtgtatcagaagggataggtatcttgcctcctttctctcaggcagtgcaaatcaccctgtggaaaaccgatggacaggaaggagtgttacacactgcttaccctgatttattcagtggttttgttttcattctggaaccatactatcaaatggcgacagactgttccgttccacccccgtgaagtaatcatgcaccgtgtgaatagtatcaagcaggattgctttcattgtatggagcatgaccagcgtgtgactcattctgacatttcagatcctaagaattctaagaacactactagaagcatttgttccctcctagtcaatgcttcatactttttcttgggattcttttagcccttgacattcttgtcccccaaacctgtaagtaggtgaattcctaagataagtgtgtattttcattccaggtgaaaagcaggatgtaccgagcactttattcagtgcatagctttaagccagtgttggattcactaagtggacagccagtctcccagctctctgccttccccaaaagggtcgtagtaggtcacccttctacagcagctaactagagtcctaactaatgggatccagcagggccatttctccagagggccagtatcctattaggagactcttggaattcttaggttctactcaagagtggaaggaccaatcacctctgatattctgtggaaggttttggggtcaaattctgccctctgcattctgtgcaacttgtataaaagtcaagttagtattacatgaatttggggtagggttagtgctttgaaaaaatgttgaaccggctgggcgcggtggctcacgtctgtaatcccagcactttgggaggccgaggcgggtggatcatgaggtcaggagttcgagaccagcctggccaacatagtgaaaccccatctctgctaaagatataaaaaattagcccggcgtggtggtgcacgcctgtaatcccagctactcgggaggctgaggcaggagaattgcttcaacctgggaggtggaggctgcagtgagccgagatcgcaccactgcgttccagcctgagcgacagggcaagactcagtctcaaaaaaaaaaaaaaggaaaaaaaaaagaaaaaaaaa

SEQ ID NO: 4 - DNA, 1700 bp, Homo sapiens
cgcccccggacaccgctgtccggctcccgggctgtcctcagcaagggcgcggtctggtactcgtgcgtcttttatcgcctcagtttccctccgccgactagcgcgcggggcccggttctccatcgcgcgcacggcagcctagcgcaatgaggcgggcagcactgcggctttgtgccttgggcaaagggcagcttactcctggaagaggactgactcaaggaccccagaaccccaagaaacagggaatcttccacattcatgaagttcgagataagttgcgggagatagtaggagcatccacaaactggagagaccatgtgaaggcaatggaagaaaggaaattacttcatagtttcttggctaaatcacaggatggactgcctcctaggagaatgaaggacagttatattgaagttctcttgcctttgggcagtgagcctgaattacgagagaaatatttgactgttcaaaacaccgtaagatttggcaggattcttgaggatcttgacagcttgggagttcttatttgttacatgcacaacaaaatccactccgccaagatgtctcctttatcgatagttacagccctggtggataagattgatatgtgtaagaagagcttgagcccagaacaggacattaagttcagtggccatgttagctgggtcgggaagacatccatggaagtgaagatgcaaatgttccagttacatggtgatgaattttgtcctgttttggatgcaacatttgtaatggtggctcgtgattctgaaaataaagggccggcatttgtaaatccactcatccctgaaagcccagaggaagaggagctctttagacaaggggaattgaacaaggggagaagaattgccttcagctccacgtcgttactgaaaatggcccccagcgctgaggagaggaccaccatacatgagatgtttctcagcacactggatccaaagactataagttttcggagtcgagttttaccctctaatgcagtgtggatggagaattcaaaactgaagagtttggaaatttgccaccctcaggagcggaacattttcaatcggatctttggtggtttccttatgaggaaggcatatgaacttgcgtgggctactgcttgtagctttggtggttctcgaccgtttgtggtagcagtagatgacatcatgtttcagaaacctgttgaggttggctcattgctctttctttcttcacaggtatgctttactcagaataattatattcaagtcagagtacacagtgaagtggcctccctgcaggagaagcagcatacaaccaccaatgtctttcatttcacgttcatgtcggaaaaagaagtgccattggttttcccaaaaacatatggagagtccatgttgtacttagatgggcagcggcatttcaactccatgagtggcccagcgaccttgagaaaggactaccttgtggagccctaagaacaccacatttgttgaaaactagcactctacccacagtgacgtggtatctgatgaagacctgatcgagtgtattgattttagtattgcttcgtgtcctccacacaggaggaggatgtattcagcctttaggatgatcagaaaagcagaaagagagagtggccggatggggctgaggggagaaagaattattaaacaataaatactttcaagacaattttaattgtgaaccta

SEQ ID NO: 5 - DNA, 2188 bp, Homo sapiens
tttgaagatgctgtaactcttgaagttgagctgaggcagaaaggttggaaaaatgcagccctctgggtattgtggggagggatgtgatgtagtaagagggtgttttgtggtgctaggattcccacgccaccaacttgcagctttataagagcgctaccaagaaccaccgctggggaaaaggttcttattcattgtttctgttggaatgtgatcttgctttctggattttaggaattcaggttactcagtataaaactctgagaaatcagtgtgacttagtccttcacctcctaagataaagtgaatatttctttacaaaataattcatgtccttaatgttaaagatgtaattttattttcaaaacatctataacatgactttcagaagcagttcatttttccaagattcctcacattatactagataaataataggccctcagttaatacccttcagttattgaattaatctagtttgtggaatgaggtgtatcctgccaacttccctctgctcccaagtacactctgagaggtaaaatgctctgggaaatggaacaagaatcgagtggatgctgactctgtgtgcccacctcctcaactgattgataatggttgaccttgggcaagtcacttctttcaatgcctcagttccccatctgtcaaatggggttaataatactgacctacctcacaggggtgttgttgtgaggcattgtaaatcaaagttaatagaatacttcagggtcctctgtggaggatgtcttgagccagagtttaagcctgacacacaggctttggtcctcactgagctgtctccaagactggaactacttagtgactcggcaaattttctgccccccacccctcatcaaagctgctagttcagatgttgacagtgttttcatgaatgctggaatcttactagtccagacttacttaggatgttgttggggaaggcacttgggattttctgtgtcttgcattcacagagggaggccatttcagattcaagagcattggattagggaatcgtgaggcagggatgctactgcgtatttctctctgcaggttggggattaaagttcctttccccatgggtttgaagcagactcagactgtctcaggatcaaagcaaccctcaatggttttgatttatgtcattgcttaccactccccaaccaatcccaggacagctgggtcactgtacccctttgtggtatctgtacctgggcctctccttcctcatagggaccagctgattgaataaatgtgaccaccttatttccaccccccacccccaaaagctacattggaattatttttcctagaaatgtgtataacactcagaattgggcattgatccttaaagcttcatcccattcaccgtattcaacatctgtcatctcttagtgtctgcagtctgaacctaaccttgaccttttttccctctggtttgagaaaactttggacactatttctacttggccaggtgtgggctcaagagccttactctttccatctcagtttaggggcgcagccagctcctcttcccaatagggctctttctgctttccctctccttggccctagatttgtaatccatgaaaaagcacaaggtcctggctccttgcggtcacattctggttctctgtgttttgtggactctgctctcactgttcacccagcactagcagtaccagatggttctgtggagtcctggggaatggagagagcacagtctgactccctgccaagtagccaggagttgacttgcccatggtccgctggctttcccaccacttcctacaggatgggatctaagagactcaagagctgggtttctttcagcactctgtactgtcccaaatagcaaacaaatcactttgtagccagatttctgaatggaaatgagaaattgaattctccatggacttttaggtttatgggggagttttagctgtgtttcttggttttatttcagccaaacatgtctgcttttgattttttttttaaagtataagtggtctatatatatgttcaccttttaaatgtaaatgtttaaaaagtaagcatttatgtgtttccataactgacatctgatgcagacctcattctctccccctcttctaccctcctcttttcccccttttcatactcttgtattggttctaataaatggttgcttttc

SEQ ID NO: 6 - DNA, 2325 bp, Homo sapiens
acctcattcatttctaccggtctctagtagtgcagcttcggctggtgtcatcggtgtccttcctccgctgccgcccccgcaaggcttcgccgtcatcgaggccatttccagcgacttgtcgcacgcttttctatatacttcgttccccgccaaccgcaaccattgacgccatgtcgggttattcgagtgaccgagaccgcggccgggaccgagggtttggtgcacctcgatttggaggaagtagggcagggcccttatctggaaagaagtttggaaaccctggggagaaattagttaaaaagaagtggaatcttgatgagctgcctaaatttgagaagaatttttatcaagagcaccctgatttggctaggcgcacagcacaagaggtggaaacatacagaagaagcaaggaaattacagttagaggtcacaactgcccgaagccagttctaaatttttatgaagccaatttccctgcaaatgtcatggatgttattgcaagacagaatttcactgaacccactgctattcaagctcagggatggccagttgctctaagtggattggatatggttggagtggcacagactggatctgggaaaacattgtcttatttgcttcctgccattgtccacatcaatcatcagccattcctagagagaggcgatgggcctatttgtttggtgctggcaccaactcgggaactggcccaacaggtgcagcaagtagctgctgaatattgtagagcatgtcgcttgaagtctacttgtatctacggtggtgctcctaagggaccacaaatacgtgatttggagagaggtgtggaaatctgtattgcaacacctggaagactgattgactttttagagtgtggaaaaaccaatctgagaagaacaacctaccttgtccttgatgaagcagatagaatgcttgatatgggctttgaaccccaaataaggaagattgtggatcaaataagacctgataggcaaactctaatgtggagtgcgacttggccaaaagaagtaagacagcttgctgaagatttcctgaaagactatattcatataaacattggtgcacttgaactgagtgcaaaccacaacattcttcagattgtggatgtgtgtcatgacgtagaaaaggatgaaaaacttattcgtctaatggaagagatcatgagtgagaaggagaataaaaccattgtttttgtggaaaccaaaagaagatgtgatgagcttaccagaaaaatgaggagagatgggtggcctgccatgggtatccatggtgacaagagtcaacaagagcgtgactgggttctaaatgaattcaaacatggaaaagctcctattctgattgctacagatgtggcctccagagggctagatgtggaagatgtgaaatttgtcatcaattatgactaccctaactcctcagaggattatattcatcgaattggaagaactgctcgcagtaccaaaacaggcacagcatacactttctttacacctaataacataaagcaagtgagcgaccttatctctgtgcttcgtgaagctaatcaagcaattaatcccaagttgcttcagttggtcgaagacagaggttcaggtcgttccaggggtagaggaggcatgaaggatgaccgtcgggacagatactctgcgggcaaaaggggtggatttaatacctttagagacagggaaaattatgacagaggttactctagcctgcttaaaagagattttggggcaaaaactcagaatggtgtttacagtgctgcaaattacaccaatgggagctttggaagtaattttgtgtctgctggtatacagaccagttttaggactggtaatccaacagggacttaccagaatggttatgatagcactcagcaatacggaagtaatgttccaaatatgcacaatggtatgaaccaacaggcatatgcatatcctgctactgcagctgcacctatgattggttatccaatgccaacaggatattcccaataagactttagaagtatatgtaaatgtctgtttttcataattgctctttatattgtgtgttatctgacaagatagttatttaagaaacatgggaattgcagaaatgactgcagtgcagcagtaattatggtgcactttttcgctatttaagttggatatttctctacattcctgaaacaatttttaggttttttttgtactagaaaatgcaggcagtgttttcacaaaagtaaatgtacagtgatttgaaatacaataatgaaggcaatgcatggccttccaataaaaaatatttgaagactgaaaaaaaaaa

SEQ ID NO: 7 - DNA, 536 bp, Homo sapiens
atacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctc

SEQ ID NO: 8 - DNA, 464 bp, Homo sapiens
cacactccctgttcccagaaaaaatggtctccagctgtctcgatgcacacactggtatatcccatgaagacctcatccagaacttcctgaatgctggcagctttcctgagatccagggctttctgcagctgcggggttcaggacggaagctttggaaacgctttttctgcttcttgcgccgatctggcctctattactccaccaagggcacctctaaggatccgaggcacctgcagtacgtggcagatgtgaacgagtccaacgtgtacgtggtgacgcagggccgcaagctctacgggatgcccactgacttcggtttctgtgtcaagcccaacaagcttcgaaatggccacaaggggcttcggatcttctgcagtgaagatgagcagagccgcacctgctggctggctgccttccgcctcttcaagtacggggtgcagctgtacaagaattaccagcaggca

SEQ ID NO: 9 - DNA, 554 bp, Homo sapiens
ccttccccaaaagggtcgtagtaggtcacccttctacagcagctaactagagtcctaactaatgggatccagcagggccatttctccagagggccagtatcctattaggagactcttggaattcttaggttctactcaagagtggaaggaccaatcacctctgatattctgtggaaggttttggggtcaaattctgccctctgcattctgtgcaacttgtataaaagtcaagttagtattacatgaatttggggtagggttagtgctttgaaaaaatgttgaaccggctgggcgcggtggctcacgtctgtaatcccagcactttgggaggccgaggcgggtggatcatgaggtcaggagttcgagaccagcctggccaacatagtgaaaccccatctctgctaaagatataaaaaattagcccggcgtggtggtgcacgcctgtaatcccagctactcgggaggctgaggcaggagaattgcttcaacctgggaggtggaggctgcagtgagccgagatcgcaccactgcgttccagcctgagcgacagggca

SEQ ID NO: 10 - DNA, 524 bp, Homo sapiens
ggctaaatcacaggatggactgcctcctaggagaatgaaggacagttatattgaagttctcttgcctttgggcagtgagcctgaattacgagagaaatatttgactgttcaaaacaccgtaagatttggcaggattcttgaggatcttgacagcttgggagttcttatttgttacatgcacaacaaaatccactccgccaagatgtctcctttatcgatagttacagccctggtggataagattgatatgtgtaagaagagcttgagcccagaacaggacattaagttcagtggccatgttagctgggtcgggaagacatccatggaagtgaagatgcaaatgttccagttacatggtgatgaattttgtcctgttttggatgcaacatttgtaatggtggctcgtgattctgaaaataaagggccggcatttgtaaatccactcatccctgaaagcccagaggaagaggagctctttagacaaggggaattgaacaaggggagaagaattgccttcagctcca

SEQ ID NO: 11 - DNA, 477 bp, Homo sapiens
ttcagattcaagagcattggattagggaatcgtgaggcagggatgctactgcgtatttctctctgcaggttggggattaaagttcctttccccatgggtttgaagcagactcagactgtctcaggatcaaagcaaccctcaatggttttgatttatgtcattgcttaccactccccaaccaatcccaggacagctgggtcactgtacccctttgtggtatctgtacctgggcctctccttcctcatagggaccagctgattgaataaatgtgaccaccttatttccaccccccacccccaaaagctacattggaattatttttcctagaaatgtgtataacactcagaattgggcattgatccttaaagcttcatcccattcaccgtattcaacatctgtcatctcttagtgtctgcagtctgaacctaaccttgaccttttttccctctggtttgagaaaactttggacactattt

SEQ ID NO: 12 - DNA, 642 bp, Homo sapiens
agtgcgacttggccaaaagaagtaagacagcttgctgaagatttcctgaaagactatattcatataaacattggtgcacttgaactgagtgcaaaccacaacattcttcagattgtggatgtgtgtcatgacgtagaaaaggatgaaaaacttattcgtctaatggaagagatcatgagtgagaaggagaataaaaccattgtttttgtggaaaccaaaagaagatgtgatgagcttaccagaaaaatgaggagagatgggtggcctgccatgggtatccatggtgacaagagtcaacaagagcgtgactgggttctaaatgaattcaaacatggaaaagctcctattctgattgctacagatgtggcctccagagggctagatgtggaagatgtgaaatttgtcatcaattatgactaccctaactcctcagaggattatattcatcgaattggaagaactgctcgcagtaccaaaacaggcacagcatacactttctttacacctaataacataaagcaagtgagcgaccttatctctgtgcttcgtgaagctaatcaagcaattaatcccaagttgcttcagttggtcgaagacagaggttcaggtcgttccaggggtagaggaggcatgaaggatgac

SEQ ID NO: 13 - DNA, 2199 bp, Homo sapiens
agtccgcctgctactcggtcccggcgctgggctgaggggaggggttgtcttaaaagtctctccttccccctgtaggggcggccggcgagtcccagtgagagcggagggtgccagaggtagggggccgagaaacaaagttcccggggcttcctccggggccgcggtcggggctgcgcgtttgaccgcccccctcctcgcgaaggcaatggcttccaaactcctgcgcgcggtcatcctcgggccgcccggctcgggcaagggcaccgtgtgccagaggatcgcccagaactttggtctccagcatctctccagcggccacttcttgcgggagaacatcaaggccagcaccgaagttggtgagatggcaaagcagtatatagagaaaagtcttttggttccagaccatgtgatcacacgcctaatgatgtccgagttggagaacaggcgtggccagcactggctccttgatggttttcctaggacattaggacaagccgaagccctggacaaaatctgtgaagtggatctagtgatcagtttgaatattccatttgaaacacttaaagatcgtctcagccgccgttggattcaccctcctagcggaagggtatataacctggacttcaatccacctcatgtacatggtattgatgacgtcactggtgaaccgttagtccagcaggaggatgataaacccgaagcagttgctgccaggctaagacagtacaaagacgtggcaaagccagtcattgaattatacaagagccgaggagtgctccaccaattttccggaacggagacgaacaaaatctggccctacgtttacacacttttctcaaacaagatcacacctattcagtccaaagaagcatattgaccctgcccaatggaagaaccaggaagatgtggtcattcattcaatagtgtgtgtagtattggtgctgtgtccaaattagaagctagctgaggtagcttgcagcatcttttctagttgaaatggtgaactgataggaaaacaaatgagtagaaagagttcatgaagaggccctcctctgcctttcaaaaggctggtcacctacacatgtttaaggtgtctctgcacatgtctcaagcccatcacaagaaagcaagtacagtgtggatttcaaatggtgtgtaacttcagctccagctggtttttgacagctgttgctgtggtaatatttttgacatgtgatggtgatagtctctggttctccccatccccacaaaggctgttgaaccacagcaccaggaagcctgagaatgaatcctgagggctctagcccaggctttgtcccaggctttctggtgtgtgccctcctggtaacagtgaaattgaagctacttactcatagtggttgtttctctggtcttgagtgactgtgtccacagttcatttttttccggtaggaataactccttttctacatccacgctccatagagtctctccttttcagacatcctgggatgaaagaatttggcttttttttttctttttttttttggacatctgttttcactcttaggcttttaaacaatagttattgcttttatccctctcagattctaataactgagagcgatggggctatattgaatctctgtatgcactgagaactgagctatgaagaggatcttattaaactgctggtctgactttatggattgacactgttcctttcttttattgtgaaaaaaaaaaaaaaccctgaaagtcttgggaaccccctaaagtcttttgggaatcctcaaaaagcatgggaagttaagtatttagctacataaatgttgtaagatcatatcttatgtatagaagtaataagaccatttggaattactggactaattgaatagttaaggtttctattcgggacaataaaatgtattttgaaagtgctgctaactattgatgctgacagtgtttcactcctatgagtgacccaaacatattataaatatgtggtaaagggaatggagcctgtggggttgagcagaatgttgtactagctgtgcctggactgagtataacagctttatgattatgagaaaacaaattctttattttttttttctgttccaaagattcatcctatggggtggccataaagtctagaattagatactaatattttgtcattcattataacatatcaataaaccatttgttaaaaaaaaa

SEQ ID NO: 14 - DNA, 3840 bp, Homo sapiens
gtcttcccctcgtgggccctgagcgggactgcagccagccccctggggcgccagctttggaggcccccgacagctgctctcgggagccgcctcccgacacccgagccccgccggcgcctcccgctcccggctcccggctcctggctccctccgcctcccccgcccctcgccccgccgccgaagaggccccgctcccgggtcggacgcctgggtctgccgggaagagcgatgagaggtgtctgaaggtggctattcactgagcgatggggttggacttgaaggaatgccaagagatgctgcccccacccccttaggcccgagggatcaggagctatgggaccagaggccctgtcatctttactgctgctgctcttggtggcaagtggagatgctgacatgaagggacattttgatcctgccaagtgccgctatgccctgggcatgcaggaccggaccatcccagacagtgacatctctgcttccagctcctggtcagattccactgccgcccgccacagcaggttggagagcagtgacggggatggggcctggtgccccgcagggtcggtgtttcccaaggaggaggagtacttgcaggtggatctacaacgactgcacctggtggctctggtgggcacccagggacggcatgccgggggcctgggcaaggagttctcccggagctaccggctgcgttactcccgggatggtcgccgctggatgggctggaaggaccgctggggtcaggaggtgatctcaggcaatgaggaccctgagggagtggtgctgaaggaccttgggccccccatggttgcccgactggttcgcttctacccccgggctgaccgggtcatgagcgtctgtctgcgggtagagctctatggctgcctctggagggatggactcctgtcttacaccgcccctgtggggcagacaatgtatttatctgaggccgtgtacctcaacgactccacctatgacggacataccgtgggcggactgcagtatgggggtctgggccagctggcagatggtgtggtggggctggatgactttaggaagagtcaggagctgcgggtctggccaggctatgactatgtgggatggagcaaccacagcttctccagtggctatgtggagatggagtttgagtttgaccggctgagggccttccaggctatgcaggtccactgtaacaacatgcacacgctgggagcccgtctgcctggcggggtggaatgtcgcttccggcgtggccctgccatggcctgggagggggagcccatgcgccacaacctagggggcaacctgggggaccccagagcccgggctgtctcagtgccccttggcggccgtgtggctcgctttctgcagtgccgcttcctctttgcggggccctggttactcttcagcgaaatctccttcatctctgatgtggtgaacaattcctctccggcactgggaggcaccttcccgccagccccctggtggccgcctggcccacctcccaccaacttcagcagcttggagctggagcccagaggccagcagcccgtggccaaggccgaggggagcccgaccgccatcctcatcggctgcctggtggccatcatcctgctcctgctgctcatcattgccctcatgctctggcggctgcactggcgcaggctcctcagcaaggctgaacggagggtgttggaagaggagctgacggttcacctctctgtccctggggacactatcctcatcaacaaccgcccaggtcctagagagccacccccgtaccaggagccccggcctcgtgggaatccgccccactccgctccctgtgtccccaatggctctgcctacagtggggactatatggagcctgagaagccaggcgccccgcttctgcccccacctccccagaacagcgtcccccattatgccgaggctgacattgttaccctgcagggcgtcaccgggggcaacacctatgctgtgcctgcactgcccccaggggcagtcggggatgggccccccagagtggatttccctcgatctcgactccgcttcaaggagaagcttggcgagggccagtttggggaggtgcacctgtgtgaggtcgacagccctcaagatctggttagtcttgatttcccccttaatgtgcgtaagggacaccctttgctggtagctgtcaagatcttacggccagatgccaccaagaatgccaggaatgatttcctgaaagaggtgaagatcatgtcgaggctcaaggacccaaacatcattcggctgctgggcgtgtgtgtgcaggacgaccccctctgcatgattactgactacatggagaacggcgacctcaaccagttcctcagtgcccaccagctggaggacaaggcagccgagggggcccctggggacgggcaggctgcgcaggggcccaccatcagctacccaatgctgctgcatgtggcagcccagatcgcctccggcatgcgctatctggccacactcaactttgtacatcgggacctggccacgcggaactgcctagttggggaaaatttcaccatcaaaatcgcagactttggcatgagccggaacctctatgctggggactattaccgtgtgcagggccgggcagtgctgcccatccgctggatggcctgggagtgcatcctcatggggaagttcacgactgcgagtgacgtgtgggcctttggtgtgaccctgtgggaggtgctgatgctctgtagggcccagccctttgggcagctcaccgacgagcaggtcatcgagaacgcgggggagttcttccgggaccagggccggcaggtgtacctgtcccggccgcctgcctgcccgcagggcctatatgagctgatgcttcggtgctggagccgggagtctgagcagcgaccacccttttcccagctgcatcggttcctggcagaggatgcactcaacacggtgtgaatcacacatccagctgcccctccctcagggagcgatccaggggaagccagtgacactaaaacaagaggacacaatggcacctctgcccttcccctcccgacagcccatcacctctaatagaggcagtgagactgcaggtgggctgggcccacccagggagctgatgccccttctccccttcctggacacactctcatgtccccttcctgttcttccttcctagaagcccctgtcgcccacccagctggtcctgtggatgggatcctctccaccctcctctagccatcccttggggaagggtggggagaaatataggatagacactggacatggcccattggagcacctgggccccactggacaacactgattcctggagaggtggctgcgcccccagcttctctctccctgtcacacactggaccccactggctgagaatctgggggtgaggaggacaagaaggagaggaaaatgtttccttgtgcctgctcctgtacttgtcctcagcttgggcttcttcctcctccatcacctgaaacactggacctgggggtagccccgccccagccctcagtcacccccacttcccacttgcagtcttgtagctagaacttctctaagcctatacgtttctgtggagtaaatattgggattggggggaaagagggagcaacggcccatagccttggggttggacatctctagtgtagctgccacattgatttttctataatcacttggggtttgtacatttttggggggagagacacagatttttacactaatatatggacctagcttgaggcaattttaatcccctgcactaggcaggtaataataaaggttgagttttccacaaaaaaaaaaaaaaaaaa

SEQ ID NO: 15 - DNA, 4674 bp, Homo sapiens
acaccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaagacaccaaatctggctgaatgaaataaattggtgataagtggaaaaaagagaaaaaccaatgattcataacaatgtatgtgaaagtgtaaaatagaatgttactttggaatgactataaacattaaaagaagactggaagcatacaactttgtacatttgtgggggaaaactattaattttttgcaaatggaaagatcaacagactatataatgatacatgactgacacttgtacactaggtaataaaactgattcatacagtctaatgatatcaccgctgttagggttttataaaactgcatttaaaaaaagatctatgaccagatattctcctgggtgctcctcaaaggaacactattaaggttcattgaaatgttttcaatcattgccttcccattgatccttctaacatgctgttgacatcacacctaatattcagagggaatgggcaaggtatgagggaaggaaataaaaaataaaataaataaaatagaatgacacaaatttgagttttgtgaacccctgaacagatggtcttaaggacgttatctggaactggagaaaagcagagttgagagacaattctatagattaaatcctggtaaggacaaacattgccattagaagaaaagcttcaaaatagacctgtggcagatgtcacatgagtagaatttctgcccagccttaactgcattcagaggataatatcaatgaactaaacttgaactaaaaattttttaaacaaaaagttataaatgaagacacatggttgtgaatacaatgatgtatttctttattttcacatacactctagctaaaagagcaagagtacacatcaacaaaaatggaaacaaggctttggctgaaaaaaacatgcatttgacaaatcatgttaatagctagacaagaagaaagttagctttgtaaacttctacttcatttgattcagagaaacagagcatgagttttcttaaaagtaacaagaaaaggaacaaaaaaaatgaggtttgaaatcttttaccatggcaaaacattaacatctttctcaaaaacatagagaaatctggaaaaatcaagaagataaaattctggaccagttagtgacattctttcaagcatacttgtaaaatgtttccttaaagtgttcttgggatgaaaatgattgtcatgtctccaacaacagtgaactgatgttgttccttggaataaaagtcaatccccaccttaaaaaaaaaaaaaa

SEQ ID NO: 16 - DNA, 1560 bp, Homo sapiens
ccgcacctgctggctcacctccgagccacctctgctgcgcaccgcagcctcggacctacagcccaggatactttgggacttgccggcgctcagaaacgcgcccagacggcccctccaccttttgtttgcctagggtcgccgagagcgcccggagggaaccgcctggccttcggggaccaccaattttgtctggaaccaccctcccggcgtatcctactccctgtgccgcgaggccatcgcttcactggaggggtcgatttgtgtgtagtttggtgacaagatttgcattcacctggcccaaaccctttttgtctctttgggtgaccggaaaactccacctcaagttttcttttgtggggctgccccccaagtgtcgtttgttttactgtagggtctccccgcccggcgcccccagtgttttctgagggcggaaatggccaattcgggcctgcagttgctgggcttctccatggccctgctgggctgggtgggtctggtggcctgcaccgccatcccgcagtggcagatgagctcctatgcgggtgacaacatcatcacggcccaggccatgtacaaggggctgtggatggactgcgtcacgcagagcacggggatgatgagctgcaaaatgtacgactcggtgctcgccctgtccgcggccttgcaggccactcgagccctaatggtggtctccctggtgctgggcttcctggccatgtttgtggccacgatgggcatgaagtgcacgcgctgtgggggagacgacaaagtgaagaaggcccgtatagccatgggtggaggcataattttcatcgtggcaggtcttgccgccttggtagcttgctcctggtatggccatcagattgtcacagacttttataaccctttgatccctaccaacattaagtatgagtttggccctgccatctttattggctgggcagggtctgccctagtcatcctgggaggtgcactgctctcctgttcctgtcctgggaatgagagcaaggctgggtaccgtgtaccccgctcttaccctaagtccaactcttccaaggagtatgtgtgacctgggatctccttgccccagcctgacaggctatgggagtgtctagatgcctgaaagggcctggggctgagctcagcctgtgggcagggtgccggacaaaggcctcctggtcactctgtccctgcactccatgtatagtcctcttgggttgggggtgggggggtgccgttggtgggagagacaaaaagagggagagtgtgctttttgtacagtaataaaaaataagtattgggaagcaggcttttttcccttcagggcctctgctttcctcccgtccagatccttgcagggagcttggaaccttagtgcacctacttcagttcagaacacttagcaccccactgactccactgacaattgactaaaagatgcaggtgctcgtatctcgacattcattcccacccccctcttatttaaatagctaccaaagtacttcttttttaataaaaaaataaagatttttattaggtaaaaaaaaaaaaaaaaaaaaa

SEQ ID NO: 17 - DNA, 1926 bp, Homo sapiens
ggcgggggcccggccgaggcaataagagcggcggcggcggcagcggcggcagcagctcccgcagctcctgctctggtccgcctcggcccggcggcggccatcagccccctcggcctcggctcgaggggcggggagctgcgcgcgcccctcggtccgaccgacaccctccccttcccgcccgtccgcgcgccccgcggcccgcggcccgcagtccgccccgcgcgctccttgccgaggagccgagcccgcgcccggcccgcccgcccggcgctgccccggccctcccggcccgcgtgaggccgcccgcgcccgccgccgccgcagcccggccgcgccccgccgccgccgccgccgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaagaatgaaaaaaaaaaaaaaaaaaaa

SEQ ID NO: 18 - DNA, 2633 bp, Homo sapiens
agtgggcgtggcggtgctgcccaggtgagccaccgctgcttctgcccagacacggtcgcctccacatccaggtctttgtgctcctcgcttgcctgttccttttccacgcattttccaggataactgtgactccaggcccgcaatggatgccctgcaactagcaaattcggcttttgccgttgatctgttcaaacaactatgtgaaaaggagccactgggcaatgtcctcttctctccaatctgtctctccacctctctgtcacttgctcaagtgggtgctaaaggtgacactgcaaatgaaattggacaggttcttcattttgaaaatgtcaaagatgtaccctttggatttcaaacagtaacatcggatgtaaacaaacttagttccttttactcactgaaactaatcaagcggctctacgtagacaaatctctgaatctttctacagagttcatcagctctacgaagagaccgtatgcaaaggaattggaaactgttgacttcaaagataaattggaagaaacgaaaggtcagatcaacaactcaattaaggatctcacagatggccactttgagaacattttagctgacaacagtgtgaacgaccagaccaaaatccttgtggttaatgctgcctactttgttggcaagtggatgaagaaattttctgaatcagaaacaaaagaatgtcctttcagagtcaacaagacagacaccaaaccagtgcagatgatgaacatggaggccacgttctgtatgggaaacattgacagtatcaattgtaagatcatagagcttccttttcaaaataagcatctcagcatgttcatcctactacccaaggatgtggaggatgagtccacaggcttggagaagattgaaaaacaactcaactcagagtcactgtcacagtggactaatcccagcaccatggccaatgccaaggtcaaactctccattccaaaatttaaggtggaaaagatgattgatcccaaggcttgtctggaaaatctagggctgaaacatatcttcagtgaagacacatctgatttctctggaatgtcagagaccaagggagtggccctatcaaatgttatccacaaagtgtgcttagaaataactgaagatggtggggattccatagaggtgccaggagcacggatcctgcagcacaaggatgaattgaatgctgaccatccctttatttacatcatcaggcacaacaaaactcgaaacatcattttctttggcaaattctgttctccttaagtggcatagcccatgttaagtcctccctgacttttctgtggatgccgatttctgtaaactctgcatccagagattcattttctagatacaataaattgctaatgttgctggatcaggaagccgccagtacttgtcatatgtagccttcacacagatagaccttttttttttttccaattctatcttttgtttccttttttcccataagacaatgacatacgcttttaatgaaaaggaatcacgttagaggaaaaatatttattcattatttgtcaaattgtccggggtagttggcagaaatacagtcttccacaaagaaaattcctataaggaagatttggaagctcttcttcccagcactatgctttccttctttgggatagagaatgttccagacattctcgcttccctgaaagactgaagaaagtgtagtgcatgggacccacgaaactgccctggctccagtgaaacttgggcacatgctcaggctactataggtccagaagtccttatgttaagccctggcaggcaggtgtttattaaaattctgaattttggggattttcaaaagataatattttacatacactgtatgttatagaacttcatggatcagatctggggcagcaccctataaatcaacaccttaatatgctgcaacaaaatgtagaatattcagacaaaatggatacataaagactaagtagcccataaggggtcaaaatttgctgccaaatgcgtatgccaccaacttacaaaaacacttcgttcgcagagcttttcagattgtggaatgttggataaggaattatagacctctagtagctgaaatgcaagaccccaagaggaagttcagatcttaatataaattcactttcatttttgatagctgtcccatctggtcatttggttggcactagactggtggcaggggcttctagctgacttgcacagggattctcacaatagccgatatcagaatttgtgttgaaggaacttgtctcttcatctaatatgatagcgggaaaaggagaggaaactactgcctttagaaaatataagtaaagtgattaaagtgctcacgttaccttgacacatagtttttcagtctatgggtttagttactttagatggcaagcatgtaacttatattaatagtaatttgtaaagttggttggataagctatccgtgttgcaggttcatggattacttctctataaaaaatatgtatttaccaaaaattttgtgacattccttctcccatctcttccttgacctgcattgtaaataggttcttcttgttctgagattcaatattgaatttttcctatgctattgacaataaaatattattgaactaca

SEQ ID NO: 19 - DNA, 3659 bp, Homo sapiens
ggcggcgggcaggcagcggcccggccagctatgcggggtcctgcggccgcggctggcggcacttcctggagcggcggcggcagcggcttcccgggcacctgggcgtggggagcgggggcgcgcggcgcggggcgggcggagcgagcgcgcgccatggaggtggcgggcggcgcggagcgggcgtgctgagccccggccgccggcccggcatgggcgtctcccgcgggccctccgccggccggggctagggccggatggagccgcgggacggtagccccgaggcccggagcagcgactccgagtcggcttccgcctcgtccagcggctccgagcgcgacgccggtcccgagccggacaaggcgccgcggcgactcaacaagcggcgcttcccggggctgcggctcttcgggcacaggaaagccatcaccaagtcgggcctccagcacctggccccccctccgcccacccctggggccccgtgcagcgagtcagagcggcagatccggagtacagtggactggagcgagtcagcgacatatggggagcacatctggttcgagaccaacgtgtccggggacttctgctacgttggggagcagtactgtgtagccaggatgctgcagaagtcagtgtctcgaagaaagtgcgcagcctgcaagattgtggtgcacacgccctgcatcgagcagctggagaagataaatttccgctgtaagccgtccttccgtgaatcaggctccaggaatgtccgcgagccaacctttgtacggcaccactgggtacacagacgacgccaggacggcaagtgtcggcactgtgggaagggattccagcagaagttcaccttccacagcaaggagattgtggccatcagctgctcgtggtgcaagcaggcataccacagcaaggtgtcctgcttcatgctgcagcagatcgaggagccgtgctcgctgggggtccacgcagccgtggtcatcccgcccacctggatcctccgcgcccggaggccccagaatactctgaaagcaagcaagaagaagaagagggcatccttcaagaggaagtccagcaagaaagggcctgaggagggccgctggagacccttcatcatcaggcccaccccctccccgctcatgaagcccctgctggtgtttgtgaaccccaagagtgggggcaaccagggtgcaaagatcatccagtctttcctctggtatctcaatccccgacaagtcttcgacctgagccagggagggcccaaggaggcgctggagatgtaccgcaaagtgcacaacctgcggatcctggcgtgcgggggcgacggcacggtgggctggatcctctccaccctggaccagctacgcctgaagccgccaccccctgttgccatcctgcccctgggtactggcaacgacttggcccgaaccctcaactggggtgggggctacacagatgagcctgtgtccaagatcctctcccacgtggaggaggggaacgtggtacagctggaccgctgggacctccacgctgagcccaaccccgaggcagggcctgaggaccgagatgaaggcgccaccgaccggttgcccctggatgtcttcaacaactacttcagcctgggctttgacgcccacgtcaccctggagttccacgagtctcgagaggccaacccagagaaattcaacagccgctttcggaataagatgttctacgccgggacagctttctctgacttcctgatgggcagctccaaggacctggccaagcacatccgagtggtgtgtgatggaatggacttgactcccaagatccaggacctgaaaccccagtgtgttgttttcctgaacatccccaggtactgtgcgggcaccatgccctggggccaccctggggagcaccacgactttgagccccagcggcatgacgacggctacctcgaggtcattggcttcaccatgacgtcgttggccgcgctgcaggtgggcggacacggcgagcggctgacgcagtgtcgcgaggtggtgctcaccacatccaaggccatcccggtgcaggtggatggcgagccctgcaagcttgcagcctcacgcatccgcatcgccctgcgcaaccaggccaccatggtgcagaaggccaagcggcggagcgccgcccccctgcacagcgaccagcagccggtgccagagcagttgcgcatccaggtgagtcgcgtcagcatgcacgactatgaggccctgcactacgacaaggagcagctcaaggaggcctctgtgccgctgggcactgtggtggtcccaggagacagtgacctagagctctgccgtgcccacattgagagactccagcaggagcccgatggtgctggagccaagtccccgacatgccagaaactgtcccccaagtggtgcttcctggacgccaccactgccagccgcttctacaggatcgaccgagcccaggagcacctcaactatgtgactgagatcgcacaggatgagatttatatcctggaccctgagctgctgggggcatcggcccggcctgacctcccaacccccacttcccctctccccacctcaccctgctcacccacgccccggtcactgcaaggggatgctgcaccccctcaaggtgaagagctgattgaggctgccaagaggaacgacttctgtaagctccaggagctgcaccgagctgggggcgacctcatgcaccgagacgagcagagtcgcacgctcctgcaccacgcagtcagcactggcagcaaggatgtggtccgctacctgctggaccacgcccccccagagatccttgatgcggtggaggaaaacggggagacctgtttgcaccaagcagcggccctgggccagcgcaccatctgccactacatcgtggaggccggggcctcgctcatgaagacagaccagcagggcgacactccccggcagcgggctgagaaggctcaggacaccgagctggccgcctacctggagaaccggcagcactaccagatgatccagcgggaggaccaggagacggctgtgtagcgggccgcccacgggcagcaggagggacaatgcggccaggggacgagcgccttccttgcccacctcactgccacattccagtgggacggccacggggggacctaggccccagggaaagagccccatgccgccccctaaggagccgcccagacctagggctggactcaggagctgggggggcctcacctgttcccctgaggaccccgccggacccggaggctcacagggaacaagacacggctgggttggatatgcctttgccggggttctggggcagggcgctccctggccgcagcagatgccctcccaggagtggaggggctggagagggggaggccttcgggaagaggcttcctgggccccctggtcttcggccgggtccccagcccccgctcctgccccaccccacctcctccgggcttcctcccggaaactcagcgcctgctgcacttgcctgccctgccttgcttggcacccgctccggcgaccctccccgctcccctgtcatttcatcgcggactgtgcggcctgggggtggggggcgggactctcacggtgacatgtttacagctgggtgtgactcagtaaagtggatttttttttctttaaaaaaaa

SEQ ID NO: 20 - DNA, 3936 bp, Homo sapiens
gcggccggtgggctccgcccttaaccaagatggcgatacgcgtgggaccggaaagagtttatagatttcccgtctaccctacctctgaggtgaaggtgggactgccctgtggagcccaccctttccgttatgcgcccgcgcggcgcaatgacgtaacacaggcccgcccactgcccctgttgggttcctgagtcgtgctgcgtcgacaacggtagtgacgcgtattgcctggaggatggcggacgccggcattcgccgcgtggttcccagcgacctgtatcccctcgtgctcggcttcctgcgcgataaccaactctcagaggtggccaataagttcgccaaagcgacaggagctacacagcaggatgccaatgcctcttccctcttagacatctatagcttctggctcaagtctgccaaggtcccagagcgaaagttacaggcaaatggaccagtggctaagaaagctaagaagaaggcctcatccagtgacagtgaggacagcagcgaggaggaggaggaagttcaagggcctccagcaaagaaggctgctgtacctgccaagcgagtcggtctgcctcctgggaaggctgcagccaaagcatcagagagtagcagcagtgaagagtccagtgatgatgatgatgaggaggaccaaaagaaacagcctgtccagaagggagttaagccccaagccaaggcagccaaagctcctcctaagaaggccaagagctctgattctgattctgactcaagctccgaggatgagccaccaaagaaccagaagccaaagataacacctgtgacagttaaagctcagactaaagcccctcccaaaccagctcgagcagcacctaaaatagccaatggtaaagcagccagtagcagcagtagcagcagcagcagcagtagcagtgatgactcagaggaggagaaggcagcagccacccccaagaagactgtacctaaaaagcaagttgtggccaaggccccagtgaaagcagctaccacccctacccggaagagttctagcagtgaggattcctccagtgacgaggaagaggagcaaaaaaaacccatgaaaaataaaccaggtccctacagttcagtccccccgccttctgctcccccaccaaagaagtctctgggaacccagcctcccaagaaggctgtggagaagcagcagcctgtggaaagcagtgaagacagcagtgatgagtctgattcaagttctgaagaagagaagaaacccccaactaaggcagtagtctctaaagcaaccactaaaccacctccagcaaagaaagcagcagagagctcttcagacagctcagactctgacagctctgaggatgatgaagctccttctaagccagctggtaccaccaagaattcttcaaataagccagctgtcaccaccaagtcacctgcagtgaagccagctgcagcccccaagcaacctgtgggcggtggccagaagcttctgacgagaaaggctgacagcagctccagtgaggaagagagcagctccagtgaggaggagaagacaaagaagatggtggccaccactaagcccaaggcgactgccaaagcagctctatctctgcctgccaagcaggctcctcagggtagtagggacagcagctctgattcagacagctccagcagtgaggaggaggaagagaagacatctaagtctgcagttaagaagaagccacagaaggtagcaggaggtgcagccccttccaagccagcctctgcaaagaaaggaaaggctgagagcagcaacagttcttcttctgatgactccagtgaggaagaggaagagaagctcaagggcaagggctctccaagaccacaagcccccaaggccaatggcacctctgcactgactgcccagaatggaaaagcagctaagaacagtgaggaggaggaagaagaaaagaaaaaggcggcagtggtagtttccaaatcaggttcattaaagaagcggaagcagaatgaggctgccaaggaggcagagactcctcaggccaagaagataaagcttcagacccctaacacatttccaaaaaggaagaaaggagaaaaaagggcatcatccccattccgaagggtcagggaggaggaaattgaggtggattcacgagttgcggacaactcctttgatgccaagcgaggtgcagccggagactggggagagcgagccaatcaggttttgaagttcaccaaaggcaagtcctttcggcatgagaaaaccaagaagaagcggggcagctaccggggaggctcaatctctgtccaggtcaattctattaagtttgacagcgagtgacctgaggccatcttcggtgaagcaagggtgatgatcggagactacttactttctccagtggacctgggaaccctcaggtctctaggtgagggtcttgatgaggacagaagtttagagtaggtcctaagactttacagtgtaacatcctctctggtccttttctgtgttcctagttttgtacagacttgtttttgagtgttgagtagcagggacaaaataagggaatgttattttttaagaaaattcattttcattgttgtctccttccttttctgtgaaagtcctcatactgagaaatttgtatattttatattaaatcacttactattgatttttgttgtgattttcaaaggtggattcccacagataaaatcttggctattgcccaaaacatagtaaagggtcacgtgtgactttttataataggaagaaaattctgcctttgtgagtgcacatgtccacatttcatccctccttccctcaaaaccctagagaggggcattaaagaattgttgatgtatatgcaatgtctgttaagcatgcactatgtatttcatcctcatttattgggtctgggactgaagtttttagccagcatggacctaacctactttttgggataaaattctctgttttgttacaggcaaaattctggtatggcgtgaatgccatgggtcattctgaatatatttttttctgtaattttatcattacacgatgtttgcaatacgtgctttgttttttaatttgaaagcaaacttttctactgttgaaagacattttttgacaacttgacccttcctagtattgagttctaagttgaggactgcatcttctcgttttttacagtatagagaacaaaatgacatgagtttgaaaaatacatatcacttggtattgctgtcttggttgcagtggtgatacagaattggtttcattaattcctacatggttgagaatcactgatcaagaaagtggggggaaaaaaaacaaacgttaaaacctcaatcctcagtaggaaggtagattacattaggtgaaattataggtaatctatgtatgtgctaatggggttggaaagaaccttacagagcatattacctgataaactggagtgggtttgggagaacaaactaataggattattgtgtctcctagttggtacctgggagcaattgacatgcccccttcagaaccttaactgttagtagcagtggctgtaacaacacaaaccagtgaccagagataacagcttttaggccaagctggcctgacggtatggctgcaggaagtgactgagcagtagcggtactcagccagaccaagacggagagggaagagtccacagctttctggaagctaaggcattctggtggtagaaaagtgtgccccaagccttcatggacgagttataggtcttaagattagtctcctcttgtttggattccatacttgctaaataacctgataataacctggttttccatgtaactgcctctaggaagaaaatgtactgttcatgctgacacagatatttcagtctgcatggtaaaagttctaaatcttactacaaaataataaactggctggtttataatgtg

SEQ ID NO: 21 - DNA, 3037 bp, Homo sapiens
ctcctcacaggtgtgtctctagtcctcgtggttgcctgccccactccctgccgagacgcctgccagaaaggtcacctatcctgaaccccagcaagcctgaaacagctcagccaagcaccctgcgatggaagctgcagatgcctccaggagcaacgggtcgagcccagaagccagggatgcccggagcccgtcgggccccagtggcagcctggagaatggcaccaaggctgacggcaaggatgccaagaccaccaacgggcacggcggggaggcagctgagggcaagagcctgggcagcgccctgaagccaggggaaggtaggagcgccctgttcgcgggcaatgagtggcggcgacccatcatccagtttgtcgagtccggggacgacaagaactccaactacttcagcatggactctatggaaggcaagaggtcgccgtacgcagggctccagctgggggctgccaagaagccacccgttacctttgccgaaaagggcgagctgcgcaagtccattttctcggagtcccggaagcccacggtgtccatcatggagcccggggagacccggcggaacagctacccccgggccgacacgggccttttttcacggtccaagtccggctccgaggaggtgctgtgcgactcctgcatcggcaacaagcagaaggcggtcaagtcctgcctggtgtgccaggcctccttctgcgagctgcatctcaagccccacctggagggcgccgccttccgagaccaccagctgctcgagcccatccgggactttgaggcccgcaagtgtcccgtgcatggcaagacgatggagctcttctgccagaccgaccagacctgcatctgctacctttgcatgttccaggagcacaagaatcatagcaccgtgacagtggaggaggccaaggccgagaaggagacggagctgtcattgcaaaaggagcagctgcagctcaagatcattgagattgaggatgaagctgagaagtggcagaaggagaaggaccgcatcaagagcttcaccaccaatgagaaggccatcctggagcagaacttccgggacctggtgcgggacctggagaagcaaaaggaggaagtgagggctgcgctggagcagcgggagcaggatgctgtggaccaagtgaaggtgatcatggatgctctggatgagagagccaaggtgctgcatgaggacaagcagacccgggagcagctgcatagcatcagcgactctgtgttgtttctgcaggaatttggtgcattgatgagcaattactctctccccccacccctgcccacctatcatgtcctgctggagggggagggcctgggacagtcactaggcaacttcaaggacgacctgctcaatgtatgcatgcgccacgttgagaagatgtgcaaggcggacctgagccgtaacttcattgagaggaaccacatggagaacggtggtgaccatcgctatgtgaacaactacacgaacagcttcgggggtgagtggagtgcaccggacaccatgaagagatactccatgtacctgacacccaaaggtggggtccggacatcataccagccctcgtctcctggccgcttcaccaaggagaccacccagaagaatttcaacaatctctatggcaccaaaggtaactacacctcccgggtctgggagtactcctccagcattcagaactctgacaatgacctgcccgtcgtccaaggcagctcctccttctccctgaaaggctatccctccctcatgcggagccaaagccccaaggcccagccccagacttggaaatctggcaagcagactatgctgtctcactaccggccattctacgtcaacaaaggcaacgggattgggtccaacgaagccccatgagctcctggcggaaggaacgaggcgccacacccctgctcttcctcctgaccctgctgctcttgccttctaagctactgtgcttgtctgggtgggagggagcctggtcctgcacctgccctctgcagccctctgccagcctcttgggggcagttccggcctctccgacttccccactggccacactccattcagactcctttcctgccttgtgacctcagatggtcaccatcattcctgtgctcagaggccaacccatcacaggggtgagataggttggggcctgccctaacccgccagcctcctcctctcgggctggatctgggggctagcagtgagtacccgcatggtatcagcctgcctctcccgcccacgccctgctgtctccaggcctatagacgtttctctccaaggccctatcccccaatgttgtcagcagatgcctggacagcacagccacccatctcccattcacatggcccacctcctgcttcccagaggactggccctacgtgctctctctcgtcctacctatcaatgcccagcatggcagaacctgcagcccttggccactgcagatggaaacctctcagtgtcttgacatcaccctacccaggcggtgggtctccaccacagccactttgagtctgtggtccctggagggtggcttctcctgactggcaggatgaccttagccaagatattcctctgttccctctgctgagataaagaattcccttaacatgatataatccacccatgcaaatagctactggcccagctaccatttaccatttgcctacagaatttcattcagtctacactttggcattctctctggcgatggagtgtggctgggctgaccgcaaaaggtgccttacacactgcccccaccctcagccgttgccccatcagaggctgcctcctccttctgattaccccccatgttgcatatcagggtgctcaaggattggagaggagacaaaaccaggagcagcacagtggggacatctcccgtctcaacagccccaggcctatgggggctctggaaggatgggccagcttgcaggggttggggagggagacatccagcttgggctttcccctttggaataaaccattggtctgtcaaaaaaaaaaaaaaaaaaa

SEQ ID NO: 22 - DNA, 1885 bp, Homo sapiens
cagctctagcgaaaagccgccggtatttctccatctggctctcctctacctccaggcaggctcacccgagatccccgccccgaaccccccctgcacactcggcccagcgctgttgcccccggagcggacgtttctgcagctattctgagcacaccttgacgtcggctgagggagcgggacagggtcagcggcgaaggaggcaggccccgcgcggggatctcggaagccctgcggtgcatcatgaagttccagtacaaggaggaccatccctttgagtatcggaaaaaggaaggagaaaagatccggaagaaatatccggacagggtccccgtgattgtagagaaggctccaaaagccagggtgcctgatctggacaagaggaagtacctagtgccctctgaccttactgttggccagttctacttcttaatccggaagagaatccacctgagacctgaggacgccttattcttctttgtcaacaacaccatccctcccaccagtgctaccatgggccaactgtatgaggacaatcatgaggaagactattttctgtatgtggcctacagtgatgagagtgtctatgggaaatgagtggttggaagcccagcagatgggagcacctggacttgggggtaggggaggggtgtgtgtgcgcgacatggggaaagagggtggctcccaccgcaaggagacagaaggtgaagacatctagaaacattacaccacacacaccgtcatcacattttcacatgctcaattgatattttttgctgcttcctcggcccagggagaaagcatgtcaggacagagctgttggattggctttgatagaggaatggggatgatgtaagtttacagtattcctggggtttaattgttgtgcagtttcatagatgggtcaggaggtggacaagttggggccagagatgatggcagtccagcagcaactccctgtgctcccttctctttgggcagagattctatttttgacatttgcacaagacaggtagggaaaggggacttgtggtagtggaccatacctggggaccaaaagagacccactgtaattgatgcattgtggcccctgatcttccctgtctcacacttcttttctcccatcccggttgcaatctcactcagacatcacagtaccaccccaggggtggcagtagacaacaacccagaaatttagacagggatctcttacctttggaaaataggggttaggcatgaaggtggttgtgattaagaagatggttttgttattaaatagcattaaactggaattgacaagagtgttgagcatccctgtctaacctgctctttctctttggtgccccttatctcaccccttccttggaatttaataagtctcaggcatttccaattgtagactaaaaccactcttagcatctcctctagtattttccatgtatcaggacagaggtgtcttatgtagggagggggcaagtatgaagtaaggtaattatatactactctcattcaggattcttgctcccatgctgctgtcccttcaggctcacatgcacaggaatgctacatgatggccagctgcttccctccttggttatcatccactgcagctgctagttagaaaggtttggagggatgacttttagtaaatcatggggattttattgatttattttcacttttgggattttgtggggtgggagtggggagcaggaattgcactcagacatgacatttcaattcatctctgctaatgaaaagggttctttctcttgggggaaatgtgtgtgtcagttctgtcagctgcaagttcttgtataatgaagtcaatgccatcaggccaaggaaataaaataattgcttaccttaaaaaaaaaaaaaaaaaaaa

SEQ ID NO: 23 - DNA, 5458 bp, Homo sapiens
aagcgtcggacgcggcccggcgccgagccatggagcctgagccagtggaggactgtgtgcagagcactctcgccgccctgtatccaccctttgaggcaacagcccccaccctgttgggccaggtgttccaggtggtggagaggacttatcgggaggacgcactgaggtacacgctggacttcctggtaccagccaagcacctgcttgccaaggtccagcaggaagcctgtgcccaatacagtggattcctcttcttccatgaggggtggccgctctgcctgcatgaacaggtggtggtgcagctagcagccctaccctggcaactgctgcgcccaggagacttctatctgcaggtggtgccctcagctgcccaagcaccccgactagcactcaagtgtctggcccctgggggtgggcgggtgcaggaggttcctgtgcccaatgaggcttgtgcctacctattcacacctgagtggctacaaggcatcaacaaggaccggccaacaggtcgcctcagtacctgcctactgtctgcgccctctgggattcagcggctgccctgggctgagctcatctgtccacgatttgtgcacaaagagggcctcatggttggacatcagccaagtacactgcccccagaactgccctctggacctccagggcttcccagccctccacttcctgaggaggcgctgggtacccggagtcctggggatgggcacaatgcccctgtggaaggacctgagggcgagtatgtggagctgttagaggtgacgctgcccgtgagggggagcccaacagatgctgaaggctccccaggcctctccagagtccggacggtacccacccgcaagggcgctggagggaagggccgccaccggagacaccgggcgtggatgcaccagaagggcctggggcctcggggccaggatggagcacgcccacccggcgaggggagcagcaccggagcctcccctgagtctcccccaggagctgaggctgtcccagaggcagcagtcttggaggtgtctgagcccccagcagaggctgtgggagaagcctccggatcttgccccctgaggccaggggagcttagaggaggaggaggaggaggccagggggctgaaggaccacctggtacccctcggagaacaggcaaaggaaacagaagaaagaagcgagctgcaggtcgaggggctcttagccgaggaggggacagtgccccactgagccctggggacaaggaagatgccagccaccaagaagcccttggcaatctgccctcaccaagtgagcacaagcttccagaatgccacctggttaaggaggaatatgaaggctcagggaagccagaatctgagccaaaagagctcaaaacagcaggcgagaaagagcctcagctctctgaagcctgtgggcctacagaagagggggccggagagagagagctggaggggccaggcctgctgtgtatggcaggacacacaggcccagaaggccccctgtctgacactccaacacctccgctggagactgtgcaggaaggaaaaggggacaacattccagaagaggcccttgcagtctccgtctctgatcaccctgatgtagcttgggacttgatggcatctggattcctcatcctgacgggaggggtggaccagagtgggcgagctctgctgaccattaccccaccgtgccctcctgaggagcccccaccctcccgagacacgctgaacacaactcttcattacctccactcactgctcaggcctgatctacagacactggggctgtccgtcctgctggaccttcgtcaggcacctccactgcctccagcactcattcctgccttgagccaacttcaggactcaggagatcctccccttgttcagcggctgctgattctcattcatgatgaccttccaactgaactctgtggatttcagggtgctgaggtgctgtcagagaatgatctgaaaagagtggccaagccagaggagctgcagtgggagttaggaggtcacagggacccctctcccagtcactgggtagagatacaccaggaagtggtaaggctatgtcgcctgtgccaaggtgtgctgggctcggtacggcaggccattgaggagctggagggagcagcagagccagaggaagaggaggcagtgggaatgcccaagccactgcagaaggtgctggcagatccccggctgacggcactgcagagggatgggggggccatcctgatgaggctgcgctccactcccagcagcaagctggagggccaaggcccagctacactgtatcaggaagtggacgaggccattcaccagcttgtgcgcctctccaacctgcacgtgcagcagcaagagcagcggcagtgcctgcggcgactccagcaggtgttgcagtggctctcgggcccaggggaggagcagctggcaagctttgctatgcctggggacaccttgtctgccctgcaggagacagagctgcgattccgtgctttcagcgctgaggtccaggagcgcctggcccaggcacgggaggccctggctctggaggagaatgccacctcccagaaggtgctggatatctttgaacagcggctggagcaggttgagagtggcctccatcgggccctgcggctacagcgcttcttccagcaggcacatgaatgggtggatgagggctttgctcggctggcaggagctgggccgggtcgggaggctgtgctggctgcactggccctgcggcgggccccagagcccagtgccggcaccttccaggagatgcgggccctggccctggacctgggcagcccagcagccctgcgagaatggggccgctgccaggcccgctgccaagagctagagaggaggatccagcaacacgtgggagaggaggcgagcccacggggctaccgacgacggcgggcagacggtgccagcagtggaggggcccagtgggggccccgcagcccctcgcccagcctcagctccttgctgctccccagcagccctgggccacggccagccccatcccattgctccctggccccatgtggagaggactatgaggaagagggccctgagctggctccagaagcagagggcaggcccccaagagctgtgctgatccgaggcctggaggtcaccagcactgaggtggtagacaggacgtgctcaccacgggaacacgtgctgctgggccgggctagggggccagacggaccctggggagtaggcaccccccggatggagcgcaagcgaagcatcagtgcccagcagcggctggtgtctgagctgattgcctgtgaacaagattacgtggccaccttgagtgagccagtgccaccccctgggcctgagctgacgcctgaacttcggggcacctgggctgctgccctgagtgcccgggaaaggcttcgcagcttccaccggacacactttctgcgggagcttcagggctgcgccacccaccccctacgcattggggcctgcttccttcgccacggggaccagttcagcctttatgcacagtacgtgaagcaccgacacaaactggagaatggtctggctgcgctcagtcccttaagcaagggctccatggaggctggcccttacctgccccgagccctgcagcagcctctggaacagctgactcggtatgggcggctcctggaggagctcctgagggaagctgggcctgagctcagttctgagtgccgggcccttggggctgctgtacagctgctccgggaacaagaggcccgtggcagagacctgctggccgtggaggcggtgcgtggctgtgagatagatctgaaggagcagggacagctcttgcatcgagaccccttcactgtcatctgtggccgaaagaagtgccttcgccatgtctttctcttcgagcatctcctcctgttcagcaagctcaagggccctgaaggggggtcagagatgtttgtttacaagcaggcctttaagactgctgatatggggctgacagaaaacatcggggacagcggactctgctttgagttgtggtttcggcggcggcgtgcacgagaggcatacactctgcaggcaacctcaccagagatcaaactcaagtggacaagttctattgcccagctgctgtggagacaggcagcccacaacaaggagctccgagtgcagcagatggtgtccatgggcattgggaataaacccttcctggacatcaaagcccttggggagcggacgctgagtgccctgctcactggaagagccgcccgcacccgggcctccgtggccgtgtcatcctttgagcatgccggcccctcccttcccggcctttcgccgggagcctgctccctgcctgcccgcgtcgaggaggaggcctgggatctggacgtcaagcaaatttccctggccccagaaacacttgactcttctggagatgtgtccccaggaccaagaaacagccccagcctgcaacccccccaccctgggagcagcactcccaccctggccagtcgagggatcttagggctatcccgacagagtcatgctcgagccctgagtgaccccaccacgcctctgtgacctggagaagatccagaacttgcgtgcagcttctcctctcagcacactttgggctgggatggcagtggggcataatggagccctgggcgatcgctgaatttcttccctctgcttcctggacacagaggaggtctaacgaccagagtattgccctgccaccactatctctagtctccctagcttggtgccttctcctgcaggagtcagagcagccacattgcttgccttcataccctggaggtggggaagttatccctcttccggtgctttcccatcctgggccactgtatccaggacatcactcccatgccagccctccctggcagcccatgttctcctcttttctcaccccctgactttccctgagaagaatcatctctgccaggtcaactggagtccctggtgactccattctgaggtgtcacaagcaatgaagctatgcaaacaataggagggtgtgacaggggaaccgtagactttatatatgtaattactgttattataatactattgttatattaaatgtatttactcacactttgcctctaaggagctagagtagtcctctggattaaggtgataaataacttgagcactttccctcaaccagcccttaactagaacacagaaaataaaaccaagactggaaggtcccctctacccctcccaggcccagagctagctgactgtgtatgagcctgggagaatgtgtctcctccacagtggctcccagaggttccacacactctctgaagctccttctcccacactgcacctactccttgaggctgaactggtcacagacaaactgggatccagcacagtccagcagttctcaaaatgaggtcctcaggccacagtgcgtgaga

SEQ ID NO: 24 - DNA, 4534 bp, Homo sapiens
ggcccggcccctcgaggcaccgcctttcaattagcactcgctgattggtcgctgctcgcgcggtctcctgggtgacgggaacgcggtagcctgcttggtggagaccgggtgcgcctgcgtacttcatagttcgcgtagcggctcgagcgtggagatgaagcgtattttctcactgctagaaaagacttggcttggcgcaccaatacagtttgcctggcaaaaaacatcaggaaactaccttgcagtaacaggagctgattatattgtgaaaatctttgatcgccatggtcaaaaaagaagtgaaattaacttacctggtaactgtgttgccatggattgggataaagatggagatgtcctagcagtgattgctgagaaatctagctgcatttatctttgggatgccaacacaaataagaccagccagttagacaatggcatgagggatcaaatgtctttccttctttggtcaaaagttggaagtttcctggctgttggaactgttaaaggaaatttgcttatttataatcatcagacatctcgaaagattcctgtccttggaaaacatactaagagaatcacttgtggatgttggaatgcagaaaatctgcttgctttaggtggtgaagataaaatgattacagttagtaatcaggaaggtgacacgataagacagacacaagtgagatcagagcctagcaacatgcagtttttcttgatgaagatggatgaccgaacctctgctgctgaaagcatgataagtgtggtgcttggcaagaaaactttgttttttttaaatctgaatgaaccagataacccagctgatcttgaatttcagcaggactttggcaacattgtctgctataattggtatggtgatggccgcatcatgattggtttttcatgtggacattttgtggtcatttctactcatactggagagcttggtcaagagatatttcaggctcgtaaccataaagataatctaaccagcattgcagtatcacagactcttaacaaagttgctacatgtggagataactgcattaaaatccaagacttggttgacttaaaagacatgtatgttatactcaacctggatgaggaaaataaaggattgggtaccttgtcctggactgatgatggccagttgctagcactctctacccaaaggggctcacttcatgttttcctgaccaagcttcccatacttggggatgcctgcagcacaaggattgcctatctcacctccctccttgaagtcaccgtagccaaccctgttgaaggagagctaccaatcacagtttctgttgatgtggaacccaactttgtggcagtaggtctttatcatctggctgtaggaatgaataatcgagcttggttttatgtccttggagaaaatgctgtgaaaaaattgaaagatatggagtatctgggaacagtagccagtatttgccttcattctgactatgctgctgcactttttgaaggcaaagtccagttacatttgatagaaagcgaaatcttggatgctcaagaagaacgtgagactcggcttttcccagcagtggatgataagtgccgtatcttatgccatgccttaactagtgatttcctcatctatggtacagatactggtgtcgttcagtatttctacattgaagactggcaattcgttaatgattatcgacatcctgtcagtgtgaaaaagatttttcccgacccaaatgggaccagattagttttcattgatgaaaaaagtgatggatttgtttactgtccagtcaatgacgctacctatgagattccagatttttcaccaaccattaaaggtgttctttgggaaaactggccaatggataaaggtgtatttattgcttatgatgatgataaggtgtacacttatgtctttcacaaggacactatacaaggagccaaggttattttggctggtagcaccaaagttccttttgctcataaacctttgctgctatataatggagagctgacctgccaaacacagagtggaaaagtaaacaacatctaccttagcacccatggctttctcagcaacttaaaagatacggggcctgacgaactgagaccaatgctggcacagaatttaatgctaaagaggttttctgatgcttgggaaatgtgcaggattctgaatgatgaggctgcctggaatgagttggccagagcttgtctacatcacatggaagtggagtttgcaatccgtgtttatcggagaattggaaatgttggcatagtgatgtccttggaacaaataaagggaatagaggactacaatcttttggcaggacaccttgccatgtttaccaacgattataacctggctcaggacttgtaccttgcatccagctgtcctattgctgccctggagatgagaagggatttacagcattgggacagtgctctacaactggcaaagcatttggccccagaccagataccttttatatcaaaagaatatgctattcagcttgaattcgcgggtgattatgtaaatgctttggctcattatgagaaaggaataacaggtgataataaggaacatgatgaagcttgtctggctggagtggcccagatgtccataagaatgggagacatacgtcgaggggttaaccaagccctcaagcatcccagcagggtccttaaaagagactgtggagccatattggagaatatgaagcaattttcagaagcggcccaactgtatgaaaaaggtctctactacgataaagcagcatctgtttacatccgctctaagaattgggcaaaagttggtgatcttctgccccacgtttcttctcctaagatccatttgcagtatgccaaagccaaggaagcagatggaagatacaaagaagctgttgtagcttatgaaaatgcaaaacagtggcaaagtgtaatccgcatctatctggatcacctcaataatcctgaaaaagctgtcaatattgttagagagacccagtctctggatggagccaaaatggtagccaggttttttctacagcttggtgactatgggtctgccatccagtttcttgtcatgtccaaatgcaacaatgaagctttcacactggctcagcaacacaacaaaatggaaatctatgcagatattattggttctgaagacactactaatgaagactatcaaagcattgccttatactttgaaggagaaaagagatatcttcaggctggaaaattcttcttgctgtgtggccaatattcacgagcacttaaacacttcctgaaatgcccaagctcggaagataatgtggcaatagaaatggcaattgaaactgttggtcaggccaaagatgaactgctgaccaatcagctgatagaccatctcctgggggagaacgatggcatgcctaaggatgccaagtacctgttccgcttgtacatggctctgaagcaataccgagaagctgcccagactgccatcatcattgccagagaagagcagtctgcaggcaactaccggaatgcacacgatgttctcttcagtatgtatgcagaactgaaatcccagaagatcaaaattccctccgagatggccaccaacctcatgattctgcacagctatatactagtaaagattcatgttaaaaatggagatcacatgaaaggggctcgcatgctcattcgggtggccaacaacatcagcaaatttccatcacacattgtacccatcctgacgtcaactgtgattgagtgtcacagggcaggcctgaagaactctgctttcagcttcgcagctatgttgatgaggcctgaataccgcagcaaaatagatgccaaatacaaaaagaagatcgagggaatggtcaggagacccgatatatctgagatagaagaggccacgactccatgtccattctgcaaatttcttctcccagagtgtgaactcctctgtcctggatgtaaaaacagtatcccatattgcattgcaacaggtcgacacatgttgaaagatgactggacggtgtgtccacattgtgacttccctgctctatactcagaattgaagatcatgctaaacactgaaagcacatgtcctatgtgttcagaaagattaaacgctgctcagctgaaaaagatttcagactgtacccagtacctgcgaacggaggaggaactgtgattggcacgtgcagatacaatgctcctgagaagacagcattttccacaggaggctgtttcctcccctggtggatttaagagacggtcctttctggatacagagaaatgaaacaacggtgacctctccaggtcggcactttccacttctgtacggtggcaaaacgatgacatgtaaccttgctgtttattgtactttgtatattatttcctcttcaaagtctttcttacacactctatcctctgcactgttaatagtaacctatgacataattgtaaatattcagctttttgctaacttttgtattttgaaaaactttaaaataaaattgttgactagaaaaaaaaaaaaaaa

SEQ ID NO: 25 - DNA, 4206 bp, Homo sapiens
agcggaagatgatgaagatgcccctatgataactggattttcagatgacgtccccatggtgatagcctgaaagagctttcctcactagaaaccaaatggtgtaaatattttatttgataaagatagttgatggtttattttaaaagatgcactttgagttgcaatatgttatttttatatgggccaaaaacaaaaaacaaaaaaaaaaaaaaaggaaagaaaggaatgaataaactttgtagtaatcaactgtgaacttcaaaccaggttgattttagtaacccaattgctttgatttgacattaatgtagtcttacagggctgtgcttgctgggcatgcttttacgtctgtgagataatttcggttcagtaaattggccaatctttttatttttctaagacacagaaatgtatttaataaaaacctcgagagagtgatgggtggaaccccttctccttgaaagtgtgtacagatattccattttgtttggatatagtttataggaaagtgtgtggatgtattatggcggaaggtttctttatgttattttgttaatttattgggactctgtgtaaggccaggctttagtggtcattagacaccacatgtgttatgagccccttacccatagggttgggggtgggaagagaagcatatttttttgccattccggaagcaatccatttttattcacttgtgtgtcatgtaatggtctttggcaggagagagcactgagtcattgctggagttcagttcaacagagctgcagcttgggaagccctgtaagcccacagcttcctctcttatattaattgatggaattttactgtatgtgcctctgtacaagatgtagctttgagagctacaaaatgataacactgctttattacacactggtttcattgtcattgcaaaaacttaccctggttgtgggggagagttctagatctgtgccatgatccatacactggctaatagagtacataatttttccattttccattttttgtttttacttactactgaaggatctcaaatgtaaaattatgtatttggtttgagatggccacttattgtccttaaaaatccatactgatatatgcagtcattttgaattggacagtgccttctctttttttttctcctcttcttccatctccctcaaccatgcccccacccaatctaaagagacagtgctgtacactctcatagagatagagaagatctaaaaagttgagactactcaatccagttaacaacagcaggagcactagagtttgttcatttattctctctgtaaaacaagctgtgctttttttcttctgcctttaaaatgccacccgtgtattcaaaccatggccacttgatacttatgtagaatccatcgtgggctgatgcaagccctttatttaggcttagtgttgtgggcaccaatgtcgagcatcgttgtgacttgtgctgtatgattctcactgaagaatttcctttcagccaagaagcagtgaggtctgggaatattccaaagtcatgtctctgaatatgtgtccttgacatgcaagctttgtaaaaccccatccccgcttaggtgcgaggcatcaccttctcacaagtgtttagtttcttttaaccacaagtatcattcttgggtgataatatagtttcattctacttagggattgtttagaaaacaaagaaagagccaattaaattttttagtttttgaaatttttatttatatgtatacttagatgagtattttaagctgtcgacctttagtttgccatacgggtaggactgtatttcatgttaacaactggtggtaatgataagccttcttctagcgtattttctcttctttcctgtcactttccttttttaagtttttttttttaaagactggaattttttttggctttatcttgtcttaccgtagagatttgttcaaaactctaagccctaccacctcccctttaataagctctttaaatagttgaatcattaacaacctggtgggaggcaagtcatttaattgaaccactaggaagtgtattttcttttctttttctgccaactttttggtggcatttgtaaaagctgatataaaaggctctgagatgttattttcagttattccataggcaagcctttttacagagcatatgtctccagttgccagcttgagatatttccgagcatccggttctagctaccagtacctcccaatgcttagtgcacagtactgtagactggccatcacccctctccttggaaaatgccactgtgctgtttgaaaaaaagcagccttttagggctagagtattttatataaacagaagagctaagttcctgaagactaagctagatagctgcagctatatgtaaattgtatatttttatgaacttttgaagcacacactcctgtttccctctgtgtagctttgtggggatttcatgtatatatgctgtctgaaagaatccagaggttggagtgccaatagaaaatgaaaacaaatgccttgtactacaggcagcctctgaaggtgaccagataactgtcttcactgtgaccagtcggagtccctgcttgcttgtgaagaaggggcttttgtaccttgttggagatgccacctcagaagttcacactgtgcaggaaaaaggttttattctctcctggcatacattagaatgtcagatgcctgcatccatgtggaccacgatgggcctctaaaaattggtgggcagggggtttgcttatgagttttctctggaaaccgattttactcctggatgtattgaatgccccttgagctttatgagatacgagtccacatggataaaatgttagagagtggagttctacagaggattccaggaagaggccatgtctgtgcagtcctagttccagacaggtgagaagctccaggaactactggctaccttgacaagctgggtaaatagttatcattctgggtaactggttgaaactctgacttttggacaagtaattcctggggttctgtctttggtagcatcaccagggatatttgggtgggacagacagaagacacacagctgcctgttctctcctgcccatcatgtttggcccactagatgaagctgtactcagcaatttagggaatgtaacccttctcagaactggccattttcaggggaagcttgggagagcaatagtatggtgagccccttagagatgagcgcctactccttcttggcgaatgctgccttcagatgcttaccaagtggtcactgcatctagtaagattatatttccagtacacttccttagggcagaaacaccatcctatcaggtttggtcagtcccttcttcatgaagggagtcatggggaattcctgaaaattttcttccttctgcagacagttggatgagtcccttagagaaggcatccagagacataactaaactgaatatcatcccatattgattttaggaattgactctaaaactctgtgcagaatcttgtgttgggattgtatcttgacattcctgttgtgttatttttcttaactggagtgtgtgctgcctttcaggtacaatttttgtgtaataaaagccagtgcattaagtttatatagactactttctatgcaagactgagatatggaatagataggaagagatatgtactgctgggtacatggacagtaagtgtgttttcagatggagtaccagcaccgaaaatgggttgagggaggatgggttgtatgtatgtttctgcccactaattttgagcagccatattatgaattaaatcgtcacagccaagtaataacccaagaatggtatgagtttcatgtgtaatagctcaaatggaataagcatgaatgctggagtggaccattatcctcaaatattctatgtcacttctcatttaaagactcttgttatgaactattagaaactttaggcaaaatcaaaagtatttgcggcaaaataaaggcctattctactcttatttaaagtgaaacactgtatacttgtttctctccaaagcgaaattaagtatttataatttcaattgcctcgataagtttccaagtcactgaaatctgctgaaggttttactgtattgttgcacaactttaagataatttttgtctcaatgtcaacttttttcactgaataaaaatttaactgggtcaagaaaacaaaaaaaaaaaaaaaa

SEQ ID NO: 26 - DNA, 349 bp, Homo sapiens
caagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagc

SEQ ID NO: 27 - DNA, 363 bp, Homo sapiens
atgcacacactggtatatcccatgaagacctcatccagaacttcctgaatgctggcagctttcctgagatccagggctttctgcagctgcggggttcaggacggaagctttggaaacgctttttctgcttcttgcgccgatctggcctctattactccaccaagggcacctctaaggatccgaggcacctgcagtacgtggcagatgtgaacgagtccaacgtgtacgtggtgacgcagggccgcaagctctacgggatgcccactgacttcggtttctgtgtcaagcccaacaagcttcgaaatggccacaaggggcttcggatcttctgcagtgaagatgagcagagccgcacctgctggc

SEQ ID NO: 28 - DNA, 492 bp, Homo sapiens
tcctagtcttaattaccatattcagggtacgaactggagggcttgtgtgttagcttctgaattggcaattggaggcggtagtggtcgtgcctgtgtgtatcagaagggataggtatcttgcctcctttctctcaggcagtgcaaatcaccctgtggaaaaccgatggacaggaaggagtgttacacactgcttaccctgatttattcagtggttttgttttcattctggaaccatactatcaaatggcgacagactgttccgttccacccccgtgaagtaatcatgcaccgtgtgaatagtatcaagcaggattgctttcattgtatggagcatgaccagcgtgtgactcattctgacatttcagatcctaagaattctaagaacactactagaagcatttgttccctcctagtcaatgcttcatactttttcttgggattcttttagcccttgacattcttgtcccccaaacctgtaagtaggtgaattcc

SEQ ID NO: 29 - DNA, 560 bp, Homo sapiens
ttttggatgcaacatttgtaatggtggctcgtgattctgaaaataaagggccggcatttgtaaatccactcatccctgaaagcccagaggaagaggagctctttagacaaggggaattgaacaaggggagaagaattgccttcagctccacgtcgttactgaaaatggcccccagcgctgaggagaggaccaccatacatgagatgtttctcagcacactggatccaaagactataagttttcggagtcgagttttaccctctaatgcagtgtggatggagaattcaaaactgaagagtttggaaatttgccaccctcaggagcggaacattttcaatcggatctttggtggtttccttatgaggaaggcatatgaacttgcgtgggctactgcttgtagctttggtggttctcgaccgtttgtggtagcagtagatgacatcatgtttcagaaacctgttgaggttggctcattgctctttctttcttcacaggtatgctttactcagaataattatattcaagtcagagtacacagtgaagtggcctccctgcaggag

SEQ ID NO: 30 - DNA, 605 bp, Homo sapiens
agctcctcttcccaatagggctctttctgctttccctctccttggccctagatttgtaatccatgaaaaagcacaaggtcctggctccttgcggtcacattctggttctctgtgttttgtggactctgctctcactgttcacccagcactagcagtaccagatggttctgtggagtcctggggaatggagagagcacagtctgactccctgccaagtagccaggagttgacttgcccatggtccgctggctttcccaccacttcctacaggatgggatctaagagactcaagagctgggtttctttcagcactctgtactgtcccaaatagcaaacaaatcactttgtagccagatttctgaatggaaatgagaaattgaattctccatggacttttaggtttatgggggagttttagctgtgtttcttggttttatttcagccaaacatgtctgcttttgattttttttttaaagtataagtggtctatatatatgttcaccttttaaatgtaaatgtttaaaaagtaagcatttatgtgtttccataactgacatctgatgcagacctcattctctccccctcttctaccctcctcttttccc

SEQ ID NO: 31 - DNA, 507 bp, Homo sapiens
taaatgaattcaaacatggaaaagctcctattctgattgctacagatgtggcctccagagggctagatgtggaagatgtgaaatttgtcatcaattatgactaccctaactcctcagaggattatattcatcgaattggaagaactgctcgcagtaccaaaacaggcacagcatacactttctttacacctaataacataaagcaagtgagcgaccttatctctgtgcttcgtgaagctaatcaagcaattaatcccaagttgcttcagttggtcgaagacagaggttcaggtcgttccaggggtagaggaggcatgaaggatgaccgtcgggacagatactctgcgggcaaaaggggtggatttaatacctttagagacagggaaaattatgacagaggttactctagcctgcttaaaagagattttggggcaaaaactcagaatggtgtttacagtgctgcaaattacaccaatgggagctttggaagtaattttgtgtctgctg

SEQ ID NO: 32 - DNA, 682 bp, Homo sapiens
acaaagacgtggcaaagccagtcattgaattatacaagagccgaggagtgctccaccaattttccggaacggagacgaacaaaatctggccctacgtttacacacttttctcaaacaagatcacacctattcagtccaaagaagcatattgaccctgcccaatggaagaaccaggaagatgtggtcattcattcaatagtgtgtgtagtattggtgctgtgtccaaattagaagctagctgaggtagcttgcagcatcttttctagttgaaatggtgaactgataggaaaacaaatgagtagaaagagttcatgaagaggccctcctctgcctttcaaaaggctggtcacctacacatgtttaaggtgtctctgcacatgtctcaagcccatcacaagaaagcaagtacagtgtggatttcaaatggtgtgtaacttcagctccagctggtttttgacagctgttgctgtggtaatatttttgacatgtgatggtgatagtctctggttctccccatccccacaaaggctgttgaaccacagcaccaggaagcctgagaatgaatcctgagggctctagcccaggctttgtcccaggctttctggtgtgtgccctcctggtaacagtgaaattgaagctacttactcatagtggttgtttctctggtcttgagtgactgtgt

SEQ ID NO: 33 - DNA, 385 bp, Homo sapiens
cggccgtgtggctcgctttctgcagtgccgcttcctctttgcggggccctggttactcttcagcgaaatctccttcatctctgatgtggtgaacaattcctctccggcactgggaggcaccttcccgccagccccctggtggccgcctggcccacctcccaccaacttcagcagcttggagctggagcccagaggccagcagcccgtggccaaggccgaggggagcccgaccgccatcctcatcggctgcctggtggccatcatcctgctcctgctgctcatcattgccctcatgctctggcggctgcactggcgcaggctcctcagcaaggctgaacggagggtgttggaagaggagctgacggttcacctctctgtccctggg

SEQ ID NO: 34 - DNA, 532 bp, Homo sapiens
agcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattacca

SEQ ID NO: 35 - DNA, 550 bp, Homo sapiens
ggcctgcagttgctgggcttctccatggccctgctgggctgggtgggtctggtggcctgcaccgccatcccgcagtggcagatgagctcctatgcgggtgacaacatcatcacggcccaggccatgtacaaggggctgtggatggactgcgtcacgcagagcacggggatgatgagctgcaaaatgtacgactcggtgctcgccctgtccgcggccttgcaggccactcgagccctaatggtggtctccctggtgctgggcttcctggccatgtttgtggccacgatgggcatgaagtgcacgcgctgtgggggagacgacaaagtgaagaaggcccgtatagccatgggtggaggcataattttcatcgtggcaggtcttgccgccttggtagcttgctcctggtatggccatcagattgtcacagacttttataaccctttgatccctaccaacattaagtatgagtttggccctgccatctttattggctgggcagggtctgccctagtcatcctgggaggtgcactgctctcctgttcctgtcctg

SEQ ID NO: 36 - DNA, 524 bp, Homo sapiens
acttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgcc

SEQ ID NO: 37 - DNA, 668 bp, Homo sapiens
tctttgtgctcctcgcttgcctgttccttttccacgcattttccaggataactgtgactccaggcccgcaatggatgccctgcaactagcaaattcggcttttgccgttgatctgttcaaacaactatgtgaaaaggagccactgggcaatgtcctcttctctccaatctgtctctccacctctctgtcacttgctcaagtgggtgctaaaggtgacactgcaaatgaaattggacaggttcttcattttgaaaatgtcaaagatgtaccctttggatttcaaacagtaacatcggatgtaaacaaacttagttccttttactcactgaaactaatcaagcggctctacgtagacaaatctctgaatctttctacagagttcatcagctctacgaagagaccgtatgcaaaggaattggaaactgttgacttcaaagataaattggaagaaacgaaaggtcagatcaacaactcaattaaggatctcacagatggccactttgagaacattttagctgacaacagtgtgaacgaccagaccaaaatccttgtggttaatgctgcctactttgttggcaagtggatgaagaaattttctgaatcagaaacaaaagaatgtcctttcagagtcaacaagacagacaccaaaccagtgcagatgatgaaca

SEQ ID NO: 38 - DNA, 444 bp, Homo sapiens
tggtacagctggaccgctgggacctccacgctgagcccaaccccgaggcagggcctgaggaccgagatgaaggcgccaccgaccggttgcccctggatgtcttcaacaactacttcagcctgggctttgacgcccacgtcaccctggagttccacgagtctcgagaggccaacccagagaaattcaacagccgctttcggaataagatgttctacgccgggacagctttctctgacttcctgatgggcagctccaaggacctggccaagcacatccgagtggtgtgtgatggaatggacttgactcccaagatccaggacctgaaaccccagtgtgttgttttcctgaacatccccaggtactgtgcgggcaccatgccctggggccaccctggggagcaccacgactttgagccccagcggcatgacgacggctacctcgagg

SEQ ID NO: 39 - DNA, 454 bp, Homo sapiens
tggcggacgccggcattcgccgcgtggttcccagcgacctgtatcccctcgtgctcggcttcctgcgcgataaccaactctcagaggtggccaataagttcgccaaagcgacaggagctacacagcaggatgccaatgcctcttccctcttagacatctatagcttctggctcaagtctgccaaggtcccagagcgaaagttacaggcaaatggaccagtggctaagaaagctaagaagaaggcctcatccagtgacagtgaggacagcagcgaggaggaggaggaagttcaagggcctccagcaaagaaggctgctgtacctgccaagcgagtcggtctgcctcctgggaaggctgcagccaaagcatcagagagtagcagcagtgaagagtccagtgatgatgatgatgaggaggaccaaaagaaacagcctgtccagaagggagttaagcc

SEQ ID NO: 40 - DNA, 437 bp, Homo sapiens
ggaggtgctgtgcgactcctgcatcggcaacaagcagaaggcggtcaagtcctgcctggtgtgccaggcctccttctgcgagctgcatctcaagccccacctggagggcgccgccttccgagaccaccagctgctcgagcccatccgggactttgaggcccgcaagtgtcccgtgcatggcaagacgatggagctcttctgccagaccgaccagacctgcatctgctacctttgcatgttccaggagcacaagaatcatagcaccgtgacagtggaggaggccaaggccgagaaggagacggagctgtcattgcaaaaggagcagctgcagctcaagatcattgagattgaggatgaagctgagaagtggcagaaggagaaggaccgcatcaagagcttcaccaccaatgagaaggccatcctggagcagaacttcc

SEQ ID NO: 41 - DNA, 558 bp, Homo sapiens
gacgtttctgcagctattctgagcacaccttgacgtcggctgagggagcgggacagggtcagcggcgaaggaggcaggccccgcgcggggatctcggaagccctgcggtgcatcatgaagttccagtacaaggaggaccatccctttgagtatcggaaaaaggaaggagaaaagatccggaagaaatatccggacagggtccccgtgattgtagagaaggctccaaaagccagggtgcctgatctggacaagaggaagtacctagtgccctctgaccttactgttggccagttctacttcttaatccggaagagaatccacctgagacctgaggacgccttattcttctttgtcaacaacaccatccctcccaccagtgctaccatgggccaactgtatgaggacaatcatgaggaagactattttctgtatgtggcctacagtgatgagagtgtctatgggaaatgagtggttggaagcccagcagatgggagcacctggacttgggggtaggggaggggtgtgtgtgcgcgacatggggaaagagggtggctccca

SEQ ID NO: 42 - DNA, 401 bp, Homo sapiens
gaggagcagctggcaagctttgctatgcctggggacaccttgtctgccctgcaggagacagagctgcgattccgtgctttcagcgctgaggtccaggagcgcctggcccaggcacgggaggccctggctctggaggagaatgccacctcccagaaggtgctggatatctttgaacagcggctggagcaggttgagagtggcctccatcgggccctgcggctacagcgcttcttccagcaggcacatgaatgggtggatgagggctttgctcggctggcaggagctgggccgggtcgggaggctgtgctggctgcactggccctgcggcgggccccagagcccagtgccggcaccttccaggagatgcgggccctggccctggacctgggcagcccagcagc

SEQ ID NO: 43 - DNA, 565 bp, Homo sapiens
cctggaatgagttggccagagcttgtctacatcacatggaagtggagtttgcaatccgtgtttatcggagaattggaaatgttggcatagtgatgtccttggaacaaataaagggaatagaggactacaatcttttggcaggacaccttgccatgtttaccaacgattataacctggctcaggacttgtaccttgcatccagctgtcctattgctgccctggagatgagaagggatttacagcattgggacagtgctctacaactggcaaagcatttggccccagaccagataccttttatatcaaaagaatatgctattcagcttgaattcgcgggtgattatgtaaatgctttggctcattatgagaaaggaataacaggtgataataaggaacatgatgaagcttgtctggctggagtggcccagatgtccataagaatgggagacatacgtcgaggggttaaccaagccctcaagcatcccagcagggtccttaaaagagactgtggagccatattggagaatatgaagcaattttcagaagcggcccaactgtatgaaaa

SEQ ID NO: 44 - DNA, 474 bp, Homo sapiens
agatatgtactgctgggtacatggacagtaagtgtgttttcagatggagtaccagcaccgaaaatgggttgagggaggatgggttgtatgtatgtttctgcccactaattttgagcagccatattatgaattaaatcgtcacagccaagtaataacccaagaatggtatgagtttcatgtgtaatagctcaaatggaataagcatgaatgctggagtggaccattatcctcaaatattctatgtcacttctcatttaaagactcttgttatgaactattagaaactttaggcaaaatcaaaagtatttgcggcaaaataaaggcctattctactcttatttaaagtgaaacactgtatacttgtttctctccaaagcgaaattaagtatttataatttcaattgcctcgataagtttccaagtcactgaaatctgctgaaggttttactgtattgttgcacaactttaagataa